SEQUENCE LISTING (124 sequences)

SEQ ID NO: 1 - protein, 172 aa, synthetic construct
PEEEAHKKASRRITQAHMYGKREEELEKILEEAIRELEKNGVDREEAAHRAASRLITQAHMYGKDDRELEKILEIAIRILEKNGVDREEAAHRAASRLITQAHMYGKDDRELEKILEIAIRILEKNGVDRREAAKRAASRLRTQAHMYGKHEEEEERIEEIAKRILEKNGHE

SEQ ID NO: 2 - protein, 188 aa, synthetic construct
PEEEILVKAVNEASRITEKTKDEEKIKEALERILREAAERIRENGGNEEEAILVIAVNVASRITERVKNEEEIKKALEWILRVAAELIRENGGNEEEAILVIAVNVASRITERVKNEEEIKKALEWILRVAAELIRENGGNEEEAILVIAVNVASRITKRVTNEEEIKKAQHWIKHVAKKLIKENGGT

SEQ ID NO: 3 - protein, 192 aa, synthetic construct
MEKEYIEKMKELYKKYREEGLSPEEAVQKAAEEVAKEIIEELKKEGKSPREAYREAMRVIVKFAEEQGLSKPEAVQIAAEAVAKIVVEELKKEGKSEREAAREAMRIIYEFAEEQGLSKPEAVQIAARAVAKVVVEELRKEGKPEEEALEEAREIIREFARERGLSEEEAEQVAERAVELVRRELEERDGGP

SEQ ID NO: 4 - protein, 172 aa, synthetic construct
PKEEAEREATQRIHEALETMKSPEELKKVLEEAVKLLQEQGVPPPEAAKLAATQAIHYALETMKSPEELKKVLKVAVEILKKQGVPPKEAAKLAATQAIHYALHTMKSPEELLEVLEVSVELLKEQGVPPKEAAKLAATQAEHYAEKEKKSPEEVEEVKKVVEKLLKKQGVP

SEQ ID NO: 5 - protein, 188 aa, synthetic construct
KPEEILREAVNEAYKFVLEEDDPEKIKEKLEKLLKEAAEKVKELGGDKEEAILKIAVNVATRVVLEEDDPEKIKEVLEIVLEVAAKIVKEYGGDKEEAILKIAVNVATRIVVESDDPEKIKKLLKIVLEVAAKLVEKLGGDKVEAIKKIAKNVKELVKKELKDPEKIEEKSKIIEEVEKELVKKLGGP

SEQ ID NO: 6 - protein, 192 aa, synthetic construct
PDEEAEKHIEEVVRRARENGSSEYMALQEAAFEVSRKIFEKIKRNGGDDREAAETAIEVVVRAARKGGSSEYMALQIAAFAVSRKIFEHIKRNGGDDREAAETAIEVVVRAARKGGSSEYMALQIAAFAVSRKIFEHIKRNGGDDREAAKTAKEVVVRAARKGGSSEYMAEQIAAFAVSRKEFEHRRRNGDS

SEQ ID NO: 7 - protein, 192 aa, synthetic construct
MKEKYLEEMEKIYKEARKNGLSPEEAVQVAARKVAEKIIKEEKKKGKSEEEAYLEAMRIIYEFAKKQGFSPPEAVQIAAYAVAEIIIKDEEEKGKTPKEAYKKALKVIYKFAEEQGFSPEEAVQIAAVAVASIIVKDIQEKGGSKEEAFETAYEIVKEFAEEQGFSPEEAEQIAKYAVEYNKKVEEKRNGGP

SEQ ID NO: 8 - protein, 152 aa, synthetic construct
EEEEEIEREKKRAEKEGNEEAVDALENAEELYKKRPKDPVVKKLVKEAIRAAKEGNEEIVEALHNALVLYEKHPDDPVVKKLVEEAIRAAKEGNEEIVEALHNALVLYAKYPDDPKVKKLVEEAIKAAKEGNEEEVKKIHNEIRKYAEDPSS

SEQ ID NO: 9 - protein, 188 aa, synthetic construct
EEEKILQEAANEAYKHVKEETDKEKIKEKLRELLKTAAKKVKEQGGDEKEAILQIAANVAYRVVKEETDKEKIKERLRIILKTAAELVEELGGDKKEAILQIAANVAYRVVKEENDEEKIKERLRLILSTAAELVEELGGDKKEAIETIAKNVIEHISKELKDPELIKKREEIITEVAEELVKELGGE

SEQ ID NO: 10 - protein, 196 aa, synthetic construct
PEKEKKEIEEEVKKKAEELKKKGKSLEEIVKELARYIVEVAEKAEKEGKPEIAKIVIDVAVIVIAELLKSEGKSLEEIVKLLARFIVEVAEEAEKRGLPEVAKLVIDVAVIVIAELIKSEGKDLPEILKLMARFAVEVVEEARKRGLPEVAKLVLDVARIVAEELIKSEGLDPEEVEEKMEEFEKLVEELLKERGI

SEQ ID NO: 11 - protein, 172 aa, synthetic construct
PVEEAREEANKEITEALEKGKSPEELKEVMRRARERLEKAGVPPKEAAIIVANIAITYALEKGKSPEELKEVLKVAVEILEEQGVPPKEAAIIAANIAITYALEKGKSPEELRKVMEVAVEVLEEQGVPPKEAAKIAANIAETRAEEIGKSPEELEEVRKIAEEILKKQGVP

SEQ ID NO: 12 - protein, 172 aa, synthetic construct
PKEEAEKKTTERLHKTLKNRKSPEELLKAMEEAVKELKEQGVPPPEAAKIATQQLLAEALKERKSPEELLEVMKVAVEVLKKQGVPPPEAAKIATQQLLAEALRLRKSPEELLEVMKVAVEVLKEAGVPPPEAAKIAAQQLLHEARKHEKSPEELEEVEKVVEELLKEQGIP

SEQ ID NO: 13 - protein, 176 aa, synthetic construct
PELEKTKKEVEKALKEAKKEGNEETAEAAEEALEALDDAEVSNNPEAVETVTKVAKLLLKAIKEGNEETAKAALEALKALHDAIVSKNPEAVETVTKVAELLLKAIKEGNEETAKAALEALKALHDAIVSKNPEAVEEVTKVAEKLLKAVKEGNEEEAKKALEKLKELHDRIVSKE

SEQ ID NO: 14 - protein, 160 aa, synthetic construct
DLKKKLEENIEHLEKLAEKRGDPRLKEVVKEAKKAIESGDPEQLKLAHNAIVLAIEAVERGDPRLEEVVKEALKALKSGDPHQLKLAHNAIVLAIEAVERGDPELLEVVKVALKALKSGDPEQLRKAHNAIVLYREAVEKGDPELEKEVKELLEEVKKGE

SEQ ID NO: 15 - protein, 132 aa, synthetic construct
PREEAWLTADSASIKGSKEDIEEATRRITKTNHKRQAAWLLADSASIKGSKEIIEEATRQITETNHKRQAAWLLADSASIKGSKEIIEEATRQITETNHKRQAAWLLADSASIKGNEEIIREAERQIKKTNE

SEQ ID NO: 16 - protein, 132 aa, synthetic construct
PREEAYKLISKAVEKGDPEEIEKAFKEAEKKLSPREAANLLISEAVKKGDPELIERAFELAEKALSPREAANILISEAVKKGDPELIELAFELAHKALGPEEAARILLSEAREKGDPELIKLAEELAKKARP

SEQ ID NO: 17 - protein, 180 aa, synthetic construct
PEEELERRVSQIAVEAVEAKAKGDEKKVKELIEEVKELAKELGLSPEKAFKVFVAQIAVLAVEAQEKGDEKKVKAALEVVKEAAEVLGISPEKAFKVFVAQIAVDAVEYAAKGNEKKVEAALRVAKLAAEVFGISPEKAYKVFSYQIEKLKEEYKKEGNEKKVEAADKVIKKLKEVLGLP

SEQ ID NO: 18 - protein, 204 aa, synthetic construct
PKLEENEKALEEKKKELEKIAKEGTPEDKKMAKKATELAEKLVKIAEEEKDPELLKDLVTLAVKALELAKLAAHGTPEAKKMATKALELAEKLVKIAVEEKDPELLKDLVTLAVKALELAKLAAHGTPEAKKMATKAMELAEKLVKYAEEQKDPELLKNLVVKAVKLLELAKKAADGTPEEKKEAEKEMKKIEEELKKKEENNE

SEQ ID NO: 19 - protein, 164 aa, synthetic construct
ERYEKVKEEIEKKAKEDPNNKPVKNALVLIKEAEKAKKKGLEEWAELALKIAEELLKNPENKPVDNAAILILEAIKAKEKGLEELAKVALEIAKELLENPENKAVDNAAILILEAIKAKEKGLEELAKEALETAKELLENPENKETDNKAVELIEKIRKEKKGE

SEQ ID NO: 20 - protein, 160 aa, synthetic construct
PEEEIFYRASTATQELERRGRDEEEAVEEAAREVIKHIEGRETAIFYAASTATQSLERNGRDEEEAVENAARAVIRTIEGRETAIFYAASTATQSLERNGRDEEEAVENAARAVIRTIEGREEAIFYAASTATQSLERNGRSEEEARETAKRAVDRTIET

SEQ ID NO: 21 - protein, 160 aa, synthetic construct
DEEEILRDANERIQKRLKEGLSPEEAIEEATREIAEKYPNKELAVFLAANAAIQILLREGLSPEEAIEIAAKAVAKAVPNKELAVFLAANAAIQILLREGLSPLEAIEIAAKAVAKAVPDKELAKKLTHNAAVQILKRRGLSPEEAEKIAEEAIKKAFPE

SEQ ID NO: 22 - protein, 188 aa, synthetic construct
MEEEVRKAVEEALNTYLKIKKEGGSEEEAVKEAAKIIVETVKKAGGTPEDIVRAAVALALNVYLKVRKEGGSVEEAVELAAKVLVVTVKEAGGTPEDIVRAAVALALNVYLKVEKEGGSPEEAVRLAALVLVTTVKLAGGTPEDIVRAAELLSENLERRVKEEGGSPEEAERRRELVLKTTKELAGLP

SEQ ID NO: 23 - protein, 192 aa, synthetic construct
PDEEAEKHIEEVVRRARENGSSEYEALFEASQEVASKIFEKIKRNGGDDREAAETAIEVVVRAARKGGSSEYEALFEASQAVASKIFEHIKRNGGDDREAAETAIEVVVRAARKGGSSEYEALFEASQAVASKIFEHIKRNGGDDREAAKTAKEVVVRAARKGGSSEYEAEFEASQAVKSKEEEHRRRNGDS

SEQ ID NO: 24 - protein, 192 aa, synthetic construct
MREKYLEKMKKIYEEAEKKGLSPREAVHIAAQKVAEEVIRELKEEGKSEREAYLEALRIIVEFAKEQGLSPREAIHIAAQAVAKLVIRELKEEGKSRREAYLRAMDIVYEFAREQGLSPEEAVHIASRAVAKLIIRELEEEGKSPEEATETARRIIEEFAESKGLSPEQARRIAEQTVERIVEQDRKRKGSP

SEQ ID NO: 25 - protein, 164 aa, synthetic construct
PEEEKLAKEQAEHLYDDPEEREVAEKVIIEAIKRVKEMGLPPEVALKAATQAVSLFKELEEARVAAKVILEAIKRVKEMGLPPEVALKAATQAVSLFKELEEAKVAAEVILEALKLVKEMGLPPEDALRAATQAVSKHKELEPAKEEARRLLEEILRNRKKGEP

SEQ ID NO: 26 - protein, 172 aa, synthetic construct
PVEEAIEKAEKEISEAVENKKSPEELKEVLRKAVKKLEEQGVPPKEAAVRAARLAIDVAVDVKKSPEELKEVLKVAVEILEEKGVPPKEAAVIAARLLIDVAVDVKKSPEELREALEVAVEILEEQGVPPKEAARIASELLLDLVRDHSKSPEEEREAEEVAREVRKKRGVP

SEQ ID NO: 27 - protein, 132 aa, synthetic construct
PKPRAHELLDEAVKSGDPEEIERAVREVTRLLPPREAAHLLLDVAVQEGDPELIERAVRLVTRLLSPREAAHLLVDVAVQEGDPELIERAWRDVTRLLSPEEAARILKDVAEQEGDPELLERARELPKEAEP

SEQ ID NO: 28 - protein, 264 aa, synthetic construct
PEPEIKKLIEEAKKKAKEEADKIVKEKKKKKAPEEEIVKEVVESLVRKLQEVLEKLVEILKENGVPPEEIIERVKEVAHELAKEAAKILVEVLSKHGVPRELVVKAVVLFLVLALESLLRLLVELLKEQGVPPEEILREVRETALELAKEAAKIVVEVLSKHGVPEELVVKAVVEFLVLALESLLRLLVELLKEQGVPPEEILERVREEAERLKEEALKIVKEVLSKHGVPPEDVEKAVERLKRLVEQSLERLLEEIRREQGHP

SEQ ID NO: 29 - protein, 264 aa, synthetic construct
PEEEEERHKEEAVKEAEKKAKHAVKEAKKNNEDDDEILHKLVLLLSLIIQIILLELEKILEKNGTSREEIEETIKEVAVKLAEEAAKIAVKVAKENNQEDDKILHALVLLLSLIIQIILLTLEKILEKNGTSREEIEETIKEVAVKLAEEAAKIAVKVAKENNQEDDKILHALVLLLSLIIQIILLTLEKILEKNGTSREEIEETIKKVAKELAHEASRIAEKVAKENNQEDEKIKHALELLQSLIEQIIKLTEEKIKRKNGHE

SEQ ID NO: 30 - protein, 264 aa, synthetic construct
PEEEIKEEVKKAREEAKKEAEKIVKTLKEKGVPEEEVVEAVVEHLVQKIKELLEKLVEMMKKNGVPPEEIKKEILKTAKELAKKAAEIIVEVLKKHKVPEELVVEAVVKYLVLAIEVLLRILKELLKEMGVPPEEIIKEIKETAKELAKKAAEIVVEVLKKHKVPEELVVEAVVKYLVLAIEVALRILEELLKEMGVPPEEIIKKIEETAKELLEKAEKIVREVLKKNNVPPELVEQAVEEFKKLAEQMLKQILERLRKENGHP

SEQ ID NO: 31 - protein, 264 aa, synthetic construct
MEEEIEKHVKEFKEKAKEEAKKIVETLKKEKKPEEEIVKEVVEYLVRKLQEALEELIELLKEQGVPPEEILKRVLELAKELAKELAKIIVEVLKEFGIPEEEIVKAVVEFLVLALQVALELVKEILKEQGVPKEEILKKVLELSKELAKELAKIIVEVLKEFGVPEEEIVKAVVEFLVLAVQVALELLKEILKEQGVPLEEIKKKVEEYAKELAKELEKIIKEVLKEFGVPEEEIEKAVKEFKKLFEQKLKELLEEIRKEQGEP

SEQ ID NO: 32 - protein, 256 aa, synthetic construct
PEEKELEESAKENEEEAKKYEEKAKELEKKGKKEEADFLLTLAAALKVAAQAYKAIAEAEKKKDDPSLELYEEAAELAKEAAELVKKAEEKYKKGKKEEADYYLLEAAALIVAAQALLAIAEARKKKDPDLEELSEEALELATKAAELVKKAEELWKKGKKEEADFYLLEAAALIVAAQALLAIYEAKKKKDKPALEKAEKAAELAKEAAKLVKEAYKLYKKGKKEEAKKYKEEAKKLIKEAQELLKERVLEKNNE

SEQ ID NO: 33 - protein, 256 aa, synthetic construct
PREEELEETAHRLEKRSREAKKRAEELRRNGDEESATLLELLATALSTAAQAASMRSHAEQNNGERELRLAETAYRLAKRSSEAVERAEELRRNGDEESATLLELLATALSTAAQAASMRSHAEQNNGERELRLAETAYRLAKRSSEAVERAEELRRNGDEESATLLELLATALSTAAQAASMRSHAEQNNGERELRLAEKAYRLAKESSEAVEKAEELRRNGDEDSATKLELRATELSTKAQRASMESHREQNNE

SEQ ID NO: 34 - protein, 256 aa, synthetic construct
PEKKHLETRAKKYKEEAEKLRKKAKELKKKGKEKEAHFYKTKAAALTTAAQALEKIYEARKKKDPELEELSHEAARLATEAAELVEKAEKLAKKGKEKEAEYLKLKAAALTVAAQALLKIYEARKKKDKPAEELSKKAFEYAKEAAELVEKAKELEKKGDKKEAEYYKLKAKALTVAAQALLAISEARKEKDPELEKLSTEAFELAKEAAKLVKEALKEEKKGDKKKATELKKKAKELIKKAQEKLEEYYKKKENL

SEQ ID NO: 35 - protein, 188 aa, synthetic construct
PELEKLRKKVEELAKKAKEDGDEEAAKKFKELLNAVEAAEKAEKEGLKEIAELTVKVAKKAIEAEENKDEEAAKKYLELANVLVAAYYAEREGLKEIAELTVKVVTEFLKAAERKDEEAAKKYLELANVLVAAYYAEEEGLTELAKKTVEVVKKFLKAAQNKDEKEAKKYLDEANKLLKQYHDHENGK

SEQ ID NO: 36 - protein, 188 aa, synthetic construct
PEVERLRKEVKEEADKARENNDEDSLRKWKLLANALIAAHFAHLNGLEEVARLATEVVHEAYEASRNNDEDSLRKWLLLANALIAAHFAHLNGLEEVARLATEVVHEAYEASRNNDEDSLRKWLLLANALIAAHFAHLNGLEEVAHEATRVVHEATRASEENDEESLEKWLLEANKLIATHFAHLNGE

SEQ ID NO: 37 - protein, 188 aa, synthetic construct
GEIKELKEKVTKKAEEAKKNGDEEEAQKFEELLNALEAAEKAEKEGNKEIADLTVKVVKKALEAYEKGDEEKFQKFLELANALVAAYYALKEGNKEIAELTVKVVKKAVEAYEKGDEEKFQKFLELANALVAAYFAYKRGNKELAEKLVKYVKKFLEAEEKGDEEKAQKYLDKANELVKEYEDLEKGK

SEQ ID NO: 38 - protein, 160 aa, synthetic construct
PESEKIEKLAREWEKRGGKEEAFIASVIAQLVEAAERRGSEELLKIAELAAEWLERGGREEAFIASVIAQLVEAAERRGSEELLKIAELAAEWLERGGREEAFIASVIAQLVEAAERRGSEELLRIAEEAAEWTERGGREEAFHASVRAQLVEAAERKGT

SEQ ID NO: 39 - protein, 160 aa, synthetic construct
PVAEEVEKLAEEFKKRGDEEEAELARRAAELIRRAEERGNPELKEVAELAAKALKKGDVEEAKLALRAAELIERAEERGNPELKEAALLAAKALKKGDVEEAKLALRAAELIERAEEAGNPELLKIAKEAAEALKKGKVEEAKKLIKEAEELKEKHEKGE

SEQ ID NO: 40 - protein, 160 aa, synthetic construct
EPLEKVKELAKKYEKEGNKEKAELAKIIAKLIKEAEKEGNEELLEVAKLAAKYLEEGKFEKAKLALIAAKLIKEAEKEGNEELLEAAKLAAKYLKEGKEEKAKLALIAAKLIKEAEKEGNEELKKTAKKAVEAIKKGKEEEAKKLLKEAEKIKKKREKGE

SEQ ID NO: 41 - protein, 172 aa, synthetic construct
EVEKAKKKAEKIITEALKNGKSPEELRKAMEKAVKELKKAGVPPKEAARIAAQIAITYALKDGKSPEELEEVMRIAVEVLRKQGVPPKEAARIAAQIAITYALKHGKSPEELLKVMKIAVKILREQGVPPKEAARIAAQIALYYAKKHGKSEEEIKEVEKIPEEVLKEQGVP

SEQ ID NO: 42 - protein, 172 aa, synthetic construct
PEEEAFLKASIRIQHAHEHGKREEELEKILEEAIRELEKNGVDREEAAFLAASILIQHAHEQGKDDRELEKILEIAIRILEKNGVDREEAAFLAASILIQHAHEQGKDDRELEKILEIAIRILEKNGVDRREAAFLAASILIQHAREQGKHEEEEERIEEIAKRILEKNGHE

SEQ ID NO: 43 - protein, 172 aa, synthetic construct
PEQEAFDEAAERIEEALRNGKSPEELREAMREAVKRLQEQGVPPKEAAFQAAAILIEEALEEGKSPEELREALEVAVEILKEQGVPPKEAAFQAAAILIEEALEEGKSPEELREALRIAVELLQKQGVPPKEAAKLAPKIAEQEAEKEGKSPEEIERVKKVAEEILKEQGVP

SEQ ID NO: 44 - protein, 172 aa, synthetic construct
PKEEAIEKAREKINELLENSQSPEELKEAMKEAVKLLQKQGVPPKEAAKIAAEILINAALESSQSPEELKKAMKIAVEVLKEQGVPPKEAAKIAAEILINAALESSQSPEELKTVLEIAAEILKEQGVPPKEAAKIAAEILENVAKESSKSPEEEEEVEKVVKEILKKQGIP

SEQ ID NO: 45 - protein, 172 aa, synthetic construct
PEEEAEKKAKEEITQTLANGKSPEELKEVMEKAVKELQKQGVPPKEAAKIAAKIAITQALAHGKSPEELKEVMEIAVEILKKQGVPPKEAAKIAAKIAITQALAHGKSPEQLREVMELAIEILKKEGVPPKEAAKIAAKIAEQQAKAHGKSPEQIKEVEKVAKEILKKEGIP

SEQ ID NO: 46 - protein, 160 aa, synthetic construct
NKEEAEKLLKELEKKGVPPEEAAITAAYELAEKLLREGKPLEEAAEAALLALKLAGVPPEEAAILAAYVVAEALLRLGRPEEDAAKSALLALKRAGVPPEEAARLAAYVIAEALLRLGRSEEDAEKTALLALKLAGVPEEEAEKLVKKVLEQVKKRLGAP

SEQ ID NO: 47 - protein, 160 aa, synthetic construct
LEERAKERLEELKKKGVPPEEAARRVATELVAELKEEGKSLEEIAETALLAVKLAGVPPEEAARIVATQIIAVLKQLGESEEEIAETALLALKKAGVPPEEAARIVATQIIAVLLQLGESEEEAIEAAKLALKRAGVPEEEAEKLVEKQKEAVRKQAGEP

SEQ ID NO: 48 - protein, 188 aa, synthetic construct
PEEEEVRHAIERAEEIAKEAKEKGLEELAELAKRVAEELKRAIEEGDKEKIEALKALVVALQIALEAHKKGLEELAKLAVEVVRELLEAIREGDKEKIEALKALVVALQIALEAHKKGLEELAEAALEVLKLALEAIRKGDKEKLERLKELVRDLQKALEADKKGKKEEAEEHVKRVHEEIERIKKGE

SEQ ID NO: 49 - protein, 188 aa, synthetic construct
PERERVERAIERARQIAEEAKKQGNEELARTAEEVARLMEKALKEGDEKTLKYLLNAVVALQIALEAKKEGNEELARTATEVAKLIVEAIKKGDEKTLKYLENAVVALQIALEAKKEGNEELAKLALEVAKLLVEAIKKGDEETLKKLEEAVVLLQKALEAKKEGKEEEAKKALKEAKELVEKIKKGE

SEQ ID NO: 50 - protein, 188 aa, synthetic construct
PEIEKVREEAEKLLKEAKKKKDEKTIKKLEKVLKAARIAEIAKQQGLEEVAKIAAEAAKLALEAIKKKDEKTLKKLEKVLKAARIALIAHQQGLEEIAEVAKKAAKEALKAIKKKDEKTLKKLKKVLKAAEIALIAYQNGLEEIAKKALEAAKKALEAIKKKDEKTLKELKKEIEKAEKELRDADKGK

SEQ ID NO: 51 - protein, 188 aa, synthetic construct
PEIEKIKEEAEKLKKEAEKEKDEKKLKNLKEVKEAAKIAETAKQLGLEEAAKIAAEAAKLALKAVKEKDEKKLKNLKEVIEAAKIAATAYQLGLEEIAKAAIEAAKEALKAIKKKDEKKLKNLKEVIEAAKIAATAYQNGLEEIAERAAKAAKELLKAIKKKDKKKLKELKKEIEEAKEELRKAQEGK

SEQ ID NO: 52 - protein, 188 aa, synthetic construct
PEEEKLKKLAENAKKVAKEAKKEGDKELAELSKKAVEALEEAIKKGDEETIRAARKALEAAVVAYHAKKEGDKELAELSLKAVKLLLEAREKGDEETIRAAEKALEAAVVAYYAKKEGDKELAELSLKAVKLLLEAREKGDEETIRKAEKLLELAVKAYHAKREGKEEEYKELLEKVREEIEKHEKGE

SEQ ID NO: 53 - protein, 176 aa, synthetic construct
PEVEKAKKELEKEIKEAKEKGDKELVEQAKLAKEALENAEKSGDPELEKAALELVKLLKKAEKKKDEEAVEQIKLAAEALINATYSGDPELIKAALELVKELKKAEKKKDEEAVEQIKLLAEALINATYSGDPELIKKALELLKRLEKAEKKKDEKEVEKVKKEAKELINKTYENE

SEQ ID NO: 54 - protein, 176 aa, synthetic construct
GPVEEAKKKLEELIKEAKEKGDEEKVRQLQNAKEALESAEESGDPEAEKASEELVKDQIEAIKKGDEEKVRQVQEAIEALISALYSGDPEAIKASLELVKDQREAHKKGDEEKVRQVQEAIEALISALHSGDPEAIKKSLKLYEEQLEAYKKGDEEELKKVSEEIRELIDKLHLNE

SEQ ID NO: 55 - protein, 220 aa, synthetic construct
PERERVEFAARYAHQEAKKKLEEAKRHGNEEETEEWKRETELVRRLAELAHKSDREEVLRVIFAALYALQEANKKLDEAKRHGNEEETEEWRKETELVKRLAELASKSDREEVLRVIFAALYALQEANKKLDEAKRHGNEEETEEWRKETELVKRLAELASKSDREEVLRTIFAALYNLQEANKKLDEAKRHGNEEETEHWRKRTETVKREAEKASKSDK

SEQ ID NO: 56 - protein, 220 aa, synthetic construct
EPKERVKEAAEYLLKYFKEELKKAKKKGDKKEAEHYEKLIKEVEKAKKYADEATDPEKLDVIAAALTLLEKFTKLYKEAKKKGDKEEAEKYEKLIEAVRKALEYALEATDPEKLDVIAAALTLLEKFTKLYKEAKKKGDKEEAEHYEKIIDAVLKALEYATEATDEEKLKIIAEKLKELEKLTKEYKEAKKKGDKKKAKEYKEKIEKTLKELEEAKKRKK

SEQ ID NO: 57 - protein, 200 aa, synthetic construct
PEEERKKEAEEVAAHVEQIAFIAKEQGNEEVAKLAKRLAETIKRLNEGTEEEVKRLLEAAEVAAHVLQIAFIAHEQGNEEVAKLALELAESILRLIEGTEEEVKRLLEAAEVAAHVLQIAFIAHEQGNEEVAKLALELAESILRLIEGTEEEVKELLERAEEAAHVLQHAFIATEQGNEEDAKEALRKAEEILRRNEDRR

SEQ ID NO: 58 - protein, 200 aa, synthetic construct
PREEKKKKAEEAAEYVEQIAKEAKKEGNEEIAKVAEELAKEIRKLVKSEDPEDEKKLEALVAAAYVLQIAYEAKQEGNEEIAKLALELAKEILKLVKSEDPEDEKKLEALVAAAYVLQIAYEAKREGNEEIAELALELAKEILKLVKSEDPEDEKKLEELVKAAKVLQKAYELKKEGKEEEAKKYLEEAKKILEKVKSKE

SEQ ID NO: 59 - protein, 204 aa, synthetic construct
PEELIKEAKKVLEEIEKKYKQDPNNEALKLAYKAAHNAYVAAKEAKETPEPFKELLKEAVKVTLEIAKKLAEDPENEALKLAVIASYNTLHAAKAAEETPEPFKELLKEAVKVTLEIAKKLAEKPENEALKLAVIASYNTLHAAKNAEEAPPPFKEKLSEAVKETLKYAKKLAEKPEDEELKKKVEETYNKLLELEKEETTPPP

SEQ ID NO: 60 - protein, 188 aa, synthetic construct
PEHVIKRAVMEAVNRAETEKRNGGDEEEALRLAAEVVIHVVEEEGGSEEWVIKAAVMVAVNLAETEKRNGGDKEEALRLAAEVVINVVENSGGSEEWVIKAAVMVAVNLAETEKRNGGDKEEALRLAAEVVINVVENSGGSEEWVEKAAVMVAVNLAETEKRNGGDEEEAERLAEEVRRNVVERSGGS

SEQ ID NO: 61 - protein, 188 aa, synthetic construct
PEEKIREAAQEALNRANEIEKEGGSPEEAIEVAAEILIERVKKAGGTEEDIVDAAVRVAVNLANKIRQNGGSREEAIKLAAEVLIEVVKKAGGTEEDIVDAAVRVAVNLANKILQNGGSREEAIESAALVVIITVKRAGGTEEDIRDAAERVAKNLKEKIKENGGSPEEAEKYAKEVRETTKKRAGLP

SEQ ID NO: 62 - protein, 192 aa, synthetic construct
DKEEYLEEMKKIYEEARKEGKTPEEAVLTAAKRVANKIFVDLRKQGKSEEEAAKKAMEIIYEFAKEQGLSPEEAVKIAAKAVANVVFVHLREQGKSIKEAAKEAARIIVEFAKEQGLSPEEAVKIAAKAVANIVFVYKRDEGKSEEEAKKEAKEIIYELAEEEGLSEEEAKKIAEEAVEEVVKRFRKSRGEP

SEQ ID NO: 63 - protein, 232 aa, synthetic construct
EKFEKEKKELKKEAEKLKKEAKKAKKEGNKEEAKALEEAVEALELAVDVIDNAEEKNDPEAAEEAAKLAKEAVELAEKAVKAIKEGNKEEAKALELAVEALELALWVIYNAVIKNDPEAAEEAAKLAKEAVELAKKAVKAIKEGNKEEAKALELAVEALELALWVIYNAVIKNDPEAAEKAAKLAEEAVEYAKKAVKAIKQGDEKKAKKLKEKVKKLLEELKKLYNELLEGK

SEQ ID NO: 64 - protein, 236 aa, synthetic construct
PEEEKRTKHLEEVAETVRERAKEVIDKDNDEEVKNLATWAEEAAHTVEEALKTARNDTGKEEKERLDHLVEVASTVIERAIEVIEKDNDKEVLNLATWAIEAAVTVLEALRTARNDTGKEEKERLDHLVEVASTVIERAIEVIEKDNDKEVLNLATWAIEAAVTVLEALRTARNDTGKEEKERLDHLVEEASRVIEEAIKEIEKDNDKRVLNLATWKIEKAVEELKRLRTERNDKN

SEQ ID NO: 65 - protein, 236 aa, synthetic construct
EEKEKKEKELEKLVKKVEELMKKLLEKDPTPENKILEKIFVESAKEVLKAYKKALSSPDEEAKKKLEELVEVAKEVVEKALELLKQDPNEEVLLLSTILVASAVEVLKALKHALSLPDEEKKKKLEKLVEVAKEVVELALELLKQDPNEEVLLLSTILVASAVEVLKALKHALSLPDEEKKKELEKLVEEAKKVVEEALKKLKENPNEETLKEAVIKTSEAVEKLKKKIKEIESEE

SEQ ID NO: 66 - protein, 172 aa, synthetic construct
PEEKAEEEAKKMITETYQNGKSPEELKKAMKEAVKELQKQGVPPKEAAKIAAEIAITTAYQDGKSPEELKKVLELAKEILEEAGVPPKEAAKIAAEIAITTAYQDGKSPEELKKVLKVAVELLKKTGVPPKEAAKIAAEIARRVAKQDGKSPEEEKEVEKVVKEILEEEGIP

SEQ ID NO: 67 - protein, 172 aa, synthetic construct
PEEEAYRKAQKRIHTAHEHGKREEELEKILEEAIRELEKNGVDREEAAYRAAQILIHTAHEQGKDDRELEKILEIAIRILEKNGVDREEAAYRAAQILIHTAHEQGKDDRELEKILEIAIRILEKNGVDRREAAYRAAQILRHTAREQGKHEEEEERIEEIAKRILEKNGHE

SEQ ID NO: 68 - protein, 172 aa, synthetic construct
PKEKAYKEAQDRIVEALKKGKSPEELKEELKKAVERLKKQGVPPKEAAEIAAETAIRVALEEGKSPEELKEVLEVAVEILEEQGVPPKEAAEIAAETAIRVALLEGKSPEELRKVLKVAVEVLEKQGVPPKEAAEIAADTALEYAKKEGKSPEEEEEVEKVVKEIKKEKGVP

SEQ ID NO: 69 - protein, 220 aa, synthetic construct
PEREKVEKLARNAHLWAKKKLEEAKRHGNEEETEEWKRETELVRRLAELAHKSDREEVLKVIKLALNALLWANKKLDEAKRHGNEEETEEWRKETELVKRLAELASKSDREEVLKVIKLALNALLWANKKLDEAKRHGNEEETEEWRKETELVKRLAELASKSDREEVLKTIKLALNNLLWANKKLDEAKRHGNEEETEHWRKRTETVKREAEKASKSDK

SEQ ID NO: 70 - protein, 220 aa, synthetic construct
ELKEKVKELAENLLEILKKKLKEAKEKGDKKEAKFYEELIKAVEKAKEYAEKATSPVQLKVIALALNLLQHLIEAYEEAKKKGDKEEAKEYEELLKAVLKALKAASKATSPVQLKVIALALNLLQILIELLKEAKKKGDKEEAKHYEEILKAVLKALKYAHKATSPVQLKKIAKALNELQILIEELKKAKKKGDKKKAKEKKEEIKKVLKELKEAKKLKK

SEQ ID NO: 71 - protein, 160 aa, synthetic construct
KPEEVRERATQTIRELLEKGKSPEEAIEEATKEVLKEVPDKELAVRETAAQAIQILLSQGHSPEEAIEIATKAVLEAVPDKELAVRETAAQAIQILLSQGHSPEEAIEITTKAVLEAVPDKELARRETRRQAIQILRSRGLSPEEAEEITEKAVEAAFPE

SEQ ID NO: 72 - protein, 160 aa, synthetic construct
DEQEIEEAATQLIAERLKQGLSPEEAIKTTAKELAEKFPDKELVLELVATQAIAVLLQQGLSPEEAIKITAKALAEAFPDKELVLRLVATQAIAVLLQEGLSPKEAIKITAKALAEAFPDKELVKKLVHKQAIHVLKQQGLSPEEAEKIAEEAIKEAFPE

SEQ ID NO: 73 - protein, 306 aa, synthetic construct
DEEREKLKEKLKEVLRRAKEAKKKGDKEKLIELAYEAAALAAWIIHKDSNDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIITTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIITTDGDDEEIVELAKEALKLVKEAAEEAEKQGDEELREKLRYLSEAVREWIERNDGDD

SEQ ID NO: 74 - protein, 282 aa, synthetic construct
PEEEILVKAVNEASRITEKTKDEEKIKEALERILREAAERIRENGGNEEEAILVIAVNVASRITERVKNEEEIKKALEWILRVAAELIRENGGNEEEAILVIAVNVASRITERVKNEEEIKKALEWILRVAAELIRENGGNEEEAILVIAVNVASRITERVKNEEEIKKALEWILRVAAELIRENGGNEEEAILVIAVNVASRITERVKNEEEIKKALEWILRVAAELIRENGGNEEEAILVIAVNVASRITKRVTNEEEIKKAQHWIKHVAKKLIKENGGT

SEQ ID NO: 75 - protein, 240 aa, synthetic construct
PESEKIEKLAREWEKRGGKEEAYIASVIAQLVEAAERRGSEELLKIAELAAEWLERGGREEAYIAVVIAQLVEAAERRGSEELLKIAELAAEWLERGGREEAYIAVVIAQLVEAAERRGSEELLKIAELAAEWLERGGREEAYIAVVIAQLVEAAERRGSEELLKIAELAAEWLERGGREEAYIAVVIAQLVEAAERRGSEELLRIAEEAAEWTERGGREEAYHAVVRAQLVEAAERKGT

SEQ ID NO: 76 - protein, 258 aa, synthetic construct
PEEEAHKKTWKRITAAFDFGKREEELEKILEEAIRELEKNGVDREEAAHRATWILITAAFDFGKDDRELEKILEIAIRILEKNGVDREEAAHRATWILITAAFDFGKDDRELEKILEIAIRILEKNGVDREEAAHRATWILITAAFDFGKDDRELEKILEIAIRILEKNGVDREEAAHRATWILITAAFDFGKDDRELEKILEIAIRILEKNGVDRREAAKRATWILRTAAHDFGKHEEEEERIEEIAKRILEKNGHE

SEQ ID NO: 77 - protein, 270 aa, synthetic construct
PEEKLYWEVSQIAFSAIEEQKNGGDEHVEKALKKVKKAAEELGNSEEEALYWLVSQIAFSAIELQKNGGDEYVEAALKVVKKAAEELGNSEEEALYWLVSQIAFSAIELQKNGGDEYVEAALKVVKKAAEELGNSEEEALYWLVSQIAFSAIELQKNGGDEYVEAALKVVKKAAEELGNSEEEALYWLVSQIAFSAIELQKNGGDEYVEAALKVVKKAAEELGNSEDEALHLLKSQIAFSAHELENNGGEEYVKAAEKVEKKAAEELGNE

SEQ ID NO: 78 - protein, 282 aa, synthetic construct
PEEERIKYVITVVEQIAKDAHRNGQEELAKLAERTAEEAKKATERGEEETLRIVYVIVVVLQIALEAHRNGQEELAKLALRTAEEAIKATERGEEETLRIVYVIVVVLQIALEAHRNGQEELAKLALRTAEEAIKATERGEEETLRIVYVIVVVLQIALEAHRNGQEELAKLALRTAEEAIKATERGEEETLRIVYVIVVVLQIALEAHRNGQEELAKLALRTAEEAIKATERGEEETERIVYDIVVVLQEALEAHRNGEEERAKKALDEARRRIEATERGE

SEQ ID NO: 79 - protein, 330 aa, synthetic construct
PEREKVEKLARNAHLWAKKKLEEAKRHGNEEETEEWKRETELVRRLAELAHKSDREEVLKVIKLALNALLWANKKLDEAKRHGNEEETEEWRKETELVKRLAELASKSDREEVLKVIKLALNALLWANKKLDEAKRHGNEEETEEWRKETELVKRLAELASKSDREEVLKVIKLALNALLWANKKLDEAKRHGNEEETEEWRKETELVKRLAELASKSDREEVLKVIKLALNALLWANKKLDEAKRHGNEEETEEWRKETELVKRLAELASKSDREEVLKTIKLALNNLLWANKKLDEAKRHGNEEETEHWRKRTETVKREAEKASKSDK

SEQ ID NO: 80 - protein, 258 aa, synthetic construct
PEEEAHKKANVRISVAHEYGKREEELEKILEEAIRELEKNGVDREEAAHRAANVLISVAHEYGKDDRELEKILEIAIRILEKNGVDREEAAHRAANVLISVAHEYGKDDRELEKILEIAIRILEKNGVDREEAAHRAANVLISVAHEYGKDDRELEKILEIAIRILEKNGVDREEAAHRAANVLISVAHEYGKDDRELEKILEIAIRILEKNGVDRREAAKRAANVLISVAHEYGKHEEEEERIEEIAKRILEKNGHE

SEQ ID NO: 81 - protein, 258 aa, synthetic construct
PEEEAHKKAWKRITTAHQYGKREEELEKILEEAIRELEKNGVDREEAAHRAAWILITTAHQYGKDDRELEKILEIAIRILEKNGVDREEAAHRAAWILITTAHQYGKDDRELEKILEIAIRILEKNGVDREEAAHRAAWILITTAHQYGKDDRELEKILEIAIRILEKNGVDREEAAHRAAWILITTAHQYGKDDRELEKILEIAIRILEKNGVDRREAAKRAAWILRTTAHQYGKHEEEEERIEEIAKRILEKNGHE

SEQ ID NO: 82 - protein, 258 aa, synthetic construct
PEEEAHKKAWKRIVIAHQYGKREEELEKILEEAIRELEKNGVDREEAAHRAAWILIVIAHQYGKDDRELEKILEIAIRILEKNGVDREEAAHRAAWILIVIAHQYGKDDRELEKILEIAIRILEKNGVDREEAAHRAAWILIVIAHQYGKDDRELEKILEIAIRILEKNGVDREEAAHRAAWILIVIAHQYGKDDRELEKILEIAIRILEKNGVDRREAAKRAAWILRVIAHQYGKHEEEEERIEEIAKRILEKNGHE

SEQ ID NO: 83 - protein, 258 aa, synthetic construct
PEEEATKKASERIVQAHMYGKREEELEKILEEAIRELEKNGVDREEAATRAASELIVQAHMYGKDDRELEKILEIAIRILEKNGVDREEAATRAASELIVQAHMYGKDDRELEKILEIAIRILEKNGVDREEAATRAASELIVQAHMYGKDDRELEKILEIAIRILEKNGVDREEAATRAASELIVQAHMYGKDDRELEKILEIAIRILEKNGVDRREAAKRAASELRVQAHMYGKHEEEEERIEEIAKRILEKNGHE

SEQ ID NO: 84 - protein, 258 aa, synthetic construct
PEEEAHKKAFLRIDVADDYGKREEELEKILEEVIRELEKNGVDREEAAHRAAFLLIDVADDYGKDDRELEKILEIVIRILEKNGVDREEAAHRAAFLLIDVADDYGKDDRELEKILEIVIRILEKNGVDREEAAHRAAFLLIDVADDYGKDDRELEKILEIVIRILEKNGVDREEAAHRAAFLLIDVADDYGKDDRELEKILEIVIRILEKNGVDRREAAKRAAFLLRDVADDYGKHEEEEERIEEIVKRILEKNGHE

SEQ ID NO: 85 - protein, 258 aa, synthetic construct
PEEEAHKKAMIRIDVADDYGKREEELEKILEEAIRELEKNGVDREEAAHRAAMILIDVADDYGKDDRELEKILEIAIRILEKNGVDREEAAHRAAMILIDVADDYGKDDRELEKILEIAIRILEKNGVDREEAAHRAAMILIDVADDYGKDDRELEKILEIAIRILEKNGVDREEAAHRAAMILIDVADDYGKDDRELEKILEIAIRILEKNGVDRREAAKRAAMILRDVADDYGKHEEEEERIEEIAKRILEKNGHE

SEQ ID NO: 86 - protein, 354 aa, synthetic construct
PEEEKRTKHLEEVAETVRERAKEVIDKDNDEEVKNLAVWAEEAAHTVEEALKTARNDTGKEEKERLDHLVEVASTVIERAIEVIEKDNDKEVLNLAVWAIEAAVTVLEALRTARNDTGKEEKERLDHLVEVASTVIERAIEVIEKDNDKEVLNLAVWAIEAAVTVLEALRTARNDTGKEEKERLDHLVEVASTVIERAIEVIEKDNDKEVLNLAVWAIEAAVTVLEALRTARNDTGKEEKERLDHLVEVASTVIERAIEVIEKDNDKEVLNLAVWAIEAAVTVLEALRTARNDTGKEEKERLDHLVEEASRVIEEAIKEIEKDNDKRVLNLAVWKIEKAVEELKRLRTERNDKN

SEQ ID NO: 87 - protein, 258 aa, synthetic construct
PEEEAYLKAQKRIDTAHEHGKREEELEKILEEVIRELEKNGVDREEAAYLAAQILIDTAHEQGKDDRELEKILEIVIRILEKNGVDREEAAYLAAQILIDTAHEQGKDDRELEKILEIVIRILEKNGVDREEAAYLAAQILIDTAHEQGKDDRELEKILEIVIRILEKNGVDREEAAYLAAQILIDTAHEQGKDDRELEKILEIVIRILEKNGVDRREAAYLAAQILRDTAREQGKHEEEEERIEEIVKRILEKNGHE

SEQ ID NO: 88 - protein, 288 aa, synthetic construct
PDEEAEKHIEEVVRRARENGSSEYEALFEASQEVASKIFEKIKRNGGDDREAAETAIEVVVRAARKGGSSEYEALFEASQAVASKIFEHIKRNGGDDREAAETAIEVVVRAARKGGSSEYEALFEASQAVASKIFEHIKRNGGDDREAAETAIEVVVRAARKGGSSEYEALFEASQAVASKIFEHIKRNGGDDREAAETAIEVVVRAARKGGSSEYEALFEASQAVASKIFEHIKRNGGDDREAAKTAKEVVVRAARKGGSSEYEAEFEASQAVKSKEEEHRRRNGDS

SEQ ID NO: 89 - protein, 282 aa, synthetic construct
PEHVIKRAVMEAVNRAETEKENGGDEEEALRLAAEVVIHVVEEEGGSEEWVIKAAVMVAVNLAETEKENGGDKEEALRLAAEVVINVVENSGGSEEWVIKAAVMVAVNLAETEKENGGDKEEALRLAAEVVINVVENSGGSEEWVIKAAVMVAVNLAETEKENGGDKEEALRLAAEVVINVVENSGGSEEWVIKAAVMVAVNLAETEKENGGDKEEALRLAAEVVINVVENSGGSEEWVEKAAVMVAVNLAETEKENGGDEEEAERLAEEVRRNVVERSGGS

SEQ ID NO: 90 - protein, 330 aa, synthetic construct; identical to SEQ ID NO: 79

SEQ ID NO: 91 - protein, 306 aa, synthetic construct
DEEREKLKEKLKEVLRRAKEAKKKGDKEKLIELAYEAAALAAWIIHKDSNDDEIVELAKEALKLVLEAAKEAKKNGDKEKLIKLAYLAAAVAKWIIHTDGDDDEIVELAKEALKLVLEAAKEAKKNGDKEKLIKLAKLAASVAKWIIHTDGDDDEIVELAKEALKLVLEAAKEAKKNGDKEKLIKLAKLAASVAAWIIHTDGDDDEIVELAKEALKLVLEAAKEAKKNGDKEKLIKLAYLAAAVAAWIITTDGDDEEIVELAKEALKLVKEAAEEAEKQGDEELREKLRYLSEAVREWIERNDGDD

SEQ ID NO: 92 - protein, 282 aa, synthetic construct
PEEEIVEEAVNEASEITEKTKDEEKIKEALERILREAAERIRENGGNEEEAILEIAVNVASRITERVKNEEEIKKALEWILRVAAELIRENGGNEEEAILVIAINVASRITERVKNEEEIKKALEWILRVAAELIRENGGNEEEAIVVIAINVASRITERVKNEEEIKKALEWILRVAAELIRENGGNEEEAIVEIAINVASRITERVKNEEEIKKALEWILRVAAELIRENGGNEEEAILEIAVNVASRITKRVTNEEEIKKAQHWIKHVAKKLIKENGGT

SEQ ID NO: 93 - protein, 282 aa, synthetic construct
PEEEILVKAVNEASRITEKTKDEEKIKEALERILREAAERIRENGGNEEEAILVIAVNVASRITERVKNEEEIKKALEWILRVAAELIRENGGNEEEAISVIAINVASRITERVKNEEEIKKALEWILRVAAELIRENGGNEEEAIVEIAINVASRITERVKNEEEIKKALEWILRVAAELIRENGGNEEEAIVEIAINVASRITERVKNEEEIKKALEWILRVAAELIRENGGNEEEAILEIAVNVASRITKRVTNEEEIKKAQHWIKHVAKKLIKENGGT

SEQ ID NO: 94 - protein, 282 aa, synthetic construct
PEEEILIKAVNEASRITEKTKDEEKIKEALERILREAAERIRENGGNEEEAILIIAVNVASRITERVKNEEEIKKALEWILRVAAELIRENGGNEEDAISIIAINVASRITERVKNEEEIKKALEWILRVAAELIRENGGNEEDAIVEIAINVASRITERVKNEEEIKKALEWILRVAAELIRENGGNEEDAIVEIAINVASRITERVKNEEEIKKALEWILRVAAELIRENGGNEEDAILEIAVNVASRITKRVTNEEEIKKAQHWIKHVAKKLIKENGGT

SEQ ID NO: 95 - protein, 172 aa, synthetic construct
PEEEAYTKASIKIQHAFDEGKKEEELEKILEEAIKELEKNGVDKEEAAYIAASILIQSAFDTGKDDKELEKILEIAIKILEKNGVDKEEAAYIAASILIQSAFDTGKDDKELEKILEIAIKILEKNGVDKKEAAYIAASILIQSAKDTGKHEEEEEKIEEIAKKILEKNGHE

SEQ ID NO: 96 - protein, 282 aa, synthetic construct
PEEEIKKKAVNEASRITEKTKDEEKIKEALERILREAAERIRENGGNEEEAILVIAVNVASRITERVKNEEEIKKALEWILRVAAELIRENGGNEEEAILVIAINVASRITERVKNEEEIKKALEWILRVAAELIRENGGNEEEAIVTIAINVASRITERVKNEEEIKKALEWILRVAAELIRENGGNEEEAIVEIAINVASRITERVKNEEEIKKALEWILRVAAELIRENGGNEEEAILEIAVNVASRITKRVTNEEEIKKAQHWIKHVAKKLIKENGGT

SEQ ID NO: 97 - protein, 282 aa, synthetic construct
PEEEIIIKAANEAFEIVEKTKDEEKIKEALERILREAAERIRENGGNEEEAIVLIAVNVASYIVEDVKNEEEIKKALEWILRVAAELIRENGGNEEDAIVVIAVNVASRITEDVKNEEEIKKALEWILRVAAELIRENGGNEEDAIVVIAINVASRITERVKNEEEIKKALEWILRVAAELIRENGGNEEDAIVEIAINVASRITERVKNEEEIKKALEWILRVAAELIRENGGNEEDAILEIAVNVASRITKRVTNEEEIKKAQHWIKHVAKKLIKENGGT

SEQ ID NO: 98 - protein, 408 aa, synthetic construct
DEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIITTDGDDDEIVELAKEALKLVLEAAKEAKKNGDKEKLIKLAYLAAAVAAWIIHTDGDDDEIVELAKEALKLVLEAAKEAKKNGDKEKLIKLAYLAASVAAWIITTDGDDEEIVELAKEALKLVKEAAEEAEKQGDEELREKLRYLSEAVREWIERNDGDD

SEQ ID NO: 99 - protein, 408 aa, synthetic construct
DEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIKTDGDDDEIVELAKEALKLVLEAAKEAKKNGDKEKLIKLAYLAAAVAAWIIHTDGDDDEIVELAKEALKLVLEAAKEAKKNGDKEKLIKLAYLAASVAAWIITTDGDDEEIVELAKEALKLVKEAAEEAEKQGDEELREKLRYLSEAVREWIERNDGDD

SEQ ID NO: 100 - protein, 408 aa, synthetic construct
DEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIITDGDDDEIVELAKEALKLVLEAAKEAKKNGDKEKLIKLAYLAAAVAAWIIHTDGDDDEIVELAKEALKLVLEAAKEAKKNGDKEKLIKLAYLAASVAAWIITTDGDDEEIVELAKEALKLVKEAAEEAEKQGDEELREKLRYLSEAVREWIERNDGDD

SEQ ID NO: 101 - protein, 356 aa, synthetic construct
EIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIITDGDDDEIVELAKEALKLVLEAAKEAKKNGDKEKLIKLAYLAAAVAAWIIHTDGDDDEIVELAKEALKLVLEAAKEAKKNGDKEKLIKLAYLAASVAAWIITTDGDDEEIVELAKEALKLVKEAAEEAEKQGDEELREKLRYLSEAVREWIERNDGDD

SEQ ID NO: 102 - protein, 356 aa, synthetic construct
EKVELAKEKLKEVLEEAKEAKKNGDKEKLIKKAYTAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIITDGDDDEIVELAKEALKLVLEAAKEAKKNGDKEKLIKLAYLAAAVAAWIIHTDGDDDEIVELAKEALKLVLEAAKEAKKNGDKEKLIKLAYLAASVAAWIITTDGDDEEIVELAKEALKLVKEAAEEAEKQGDEELREKLRYLSEAVREWIERNDGDD

SEQ ID NO: 103 - protein, 331 aa, synthetic construct
KEKKIKKAYTAAAEAAWKIHTDGDDDEIVEEAKEALKKVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIITDGDDDEIVELAKEALKLVLEAAKEAKKNGDKEKLIKLAYLAAAVAAWIIHTDGDDDEIVELAKEALKLVLEAAKEAKKNGDKEKLIKLAYLAASVAAWIITTDGDDEEIVELAKEALKLVKEAAEEAEKQGDEELREKLRYLSEAVREWIERNDGDD

SEQ ID NO: 104 - protein, 331 aa, synthetic construct
KEKKIEKAYRAAAEAAWKIHTDGDDDEIVEEAKEALKKVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIITDGDDDEIVELAKEALKLVLEAAKEAKKNGDKEKLIKLAYLAAAVAAWIIHTDGDDDEIVELAKEALKLVLEAAKEAKKNGDKEKLIKLAYLAASVAAWIITTDGDDEEIVELAKEALKLVKEAAEEAEKQGDEELREKLRYLSEAVREWIERNDGDD

SEQ ID NO: 105 - protein, 331 aa, synthetic construct
KEKKIEKAYRAAAEAAWKIHTDGDDDEIVEEAKEALKKVLEAAKEAKKNGDKEKLIKLAYLAAAVAAWIIHTDGDDDEIVELAKEALKLVLEAAKEAKKNGDKEKLIKLAYLAAAVAAWIIHTDGDDDEIVELAKEALKLVLEAAKEAKKNGDKEKLIKLAYLAAAVAAWIIITDGDDDEIVELAKEALKLVLEAAKEAKKNGDKEKLIKLAYLAAAVAAWIIHTDGDDDEIVELAKEALKLVLEAAKEAKKNGDKEKLIYLAYLAASVAAWIITTDGDDEEIVELAKEALKLVKEAAEEAEKQGDEELREKLRYLSEAVREWIERNDGDD

SEQ ID NO: 106 - protein, 18 aa, synthetic construct
PLPPLPPEPPEPPLPPLP

SEQ ID NO: 107 - protein, 18 aa, synthetic construct
PLPPPPPEDPEQPPKPPF

SEQ ID NO: 108 - protein, 12 aa, synthetic construct
PEDPEQPPKPPF

SEQ ID NO: 109 - protein, 12 aa, synthetic construct
PLPPLPPLPPLP

SEQ ID NO: 110 - protein, 47 aa, synthetic construct
MGSSHHHHHHHHSSGGSGGLNDIFEAQKIEWHEGGSGGSENLYFQSG

SEQ ID NO: 111 - protein, 8 aa, synthetic construct
LEHHHHHH

SEQ ID NO: 112 - protein, 104 aa, synthetic construct
LPPPPQVSSLPPLSQPYVEGLCVSLEPLPPLPPLPPLPPPEDPEQPPKPPFADEEEEEEMLLREELLKSLANKRAFKPEETSSNSDPPSPPVLNNSHPVPRSNL

SEQ ID NO: 113 - protein, 18 aa, synthetic construct
LRPLRPLRPLRPLRPLRP

SEQ ID NO: 114 - protein, 18 aa, synthetic construct
PLPPLPPLPPLPPLPPLP

SEQ ID NO: 115 - protein, 18 aa, synthetic construct
LRPLRPLRPLRPLRPLRP

SEQ ID NO: 116 - protein, 18 aa, synthetic construct
PEWPEWPEWPEWPEWPEW

SEQ ID NO: 117 - protein, 18 aa, synthetic construct
IYPIYPIYPIYPIYPIYP

SEQ ID NO: 118 - protein, 18 aa, synthetic construct
PKWPKWPKWPKWPKWPKW

SEQ ID NO: 119 - protein, 18 aa, synthetic construct
PRMPRMPRMPRMPRMPRM

SEQ ID NO: 120 - protein, 18 aa, synthetic construct
IRPIRPIRPIRPIRPIRP

SEQ ID NO: 121 - protein, 18 aa, synthetic construct
PAWPAWPAWPAWPAWPAW

SEQ ID NO: 122 - protein, 12 aa, synthetic construct
LRPLRPLRPLRP

SEQ ID NO: 123 - protein, 9 aa, synthetic construct
GAGAGAGRP

SEQ ID NO: 124 - protein, 4 aa, synthetic construct
EGFP

(19) United States
(12) Patent Application Publication
(10) Pub. No.: US 2026/0159549 A1
(43) Pub. Date: Jun. 11, 2026

(54) De novo designed modular peptide binding proteins by superhelical matching

(71) Applicants: UNIVERSITY OF WASHINGTON, Seattle, WA (US); United Kingdom Research and innovation, Swindon, Wiltshire (GB); NEW YORK UNIVERSITY, New York, NY (US)

(72) Inventors: Lansing Joseph STEWART, Seattle, WA (US); David BAKER, Seattle, WA (US); Kejia WU, Seattle, WA (US); Derrick HICKS, Seattle, WA (US); TJ BRUNETTE, Seattle, WA (US); Daniel Adriano SILVA MANZANO, Seattle, WA (US); William H. SHEFFLER, Seattle, WA (US); Inna GORESHNIK, Seattle, WA (US); Emmanuel DERIVERY, Swindon, Wiltshire (GB); Kerrie MCNALLY, Swindon, Wiltshire (GB); Gira BHABHA, New York, NY (US); Damian EKIERT, New York, NY (US); Rachel REDLER, New York, NY (US); Atty CHANG, New York, NY (US); Hua BAI, Seattle, WA (US)

(21) Appl. No.: 19/123,190

(22) PCT Filed: Oct. 24, 2023
(86) PCT No.: PCT/US2023/077575

§ 371 Date: Apr. 22, 2025

Related U.S. Application Data
(60) Provisional application No. 63/381,109, filed on Oct. 26, 2022.

(51) Int. Cl. C07K 14/00; A61K 38/00; C12N 15/11; C12N 15/85; C40B 40/10

(52) U.S. Cl. CPC C07K 14/00; A61K 38/00; C12N 15/11; C12N 15/85; C40B 40/10; C12N 2830/34

(57) ABSTRACT

The disclosure provides polypeptides that bind tripeptide repeat sequences and having an amino acid sequence at least 50%, identical to the amino acid sequence of any one of SEQ ID NO: 1-105, nucleic acids encoding such polypeptides, expression vectors including such nucleic acids, and host cells including such polypeptides, nucleic acids, and/or expression vectors.

### CROSS REFERENCE

This application claims priority to U.S. Provisional Patent Application Ser. No. 63/381,109 filed Oct. 26, 2022, incorporated by reference herein in its entirety.

### FEDERAL FUNDING STATEMENT

This invention was made with government support under Grant No. 5U19AG065156-02, awarded by the National Institute on Aging. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Sep. 26, 2023 having the file name “22-1316-WO.xml” and is 131,404 bytes in size

### SUMMARY

In one aspect, the disclosure provides polypeptides comprising or consisting of an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 1-105. In another embodiment, the disclosure provides a library, comprising a plurality of different polypeptides of any embodiment of the disclosure.

In another aspect, the disclosure provides nucleic acids encoding the polypeptide of any embodiment of the disclosure. In a further embodiment, the disclosure provides expression vectors comprising a nucleic acid of the disclosure operatively linked to a suitable control sequence. In a further embodiment, the disclosure provides host cells comprising a polypeptide, nucleic acid, or expression vector of any embodiment of the disclosure. In another embodiment, the disclosure provides pharmaceutical compositions comprising
(a) the polypeptide, nucleic acid, expression vector, and/or host cell of any embodiment herein; and
(b) pharmaceutically acceptable carrier.

The disclosure further provides methods for using the polypeptide, nucleic acid, expression vector, library, pharmaceutical composition, and/or host cell of any embodiment for any suitable purpose, including but not limited to use as synthetic peptide ligands for receptor activation.

### DESCRIPTION OF FIGURES

FIG. 1. Overview of modular peptide binder design procedure. (A) Like all repeating structures, repeat proteins and peptides form superhelices with constant axial displacement (DZ), and angular twist (w) between adjacent repeat units. For in register binding, the protein and peptide parameters must match (for some integral multiple of repeat units). (B) Construction of hash tables for privileged residue-residue interactions. Top row: classes of sidechain backbone interactions for which hash tables were built; (left) sidechain amide group of asparagine or glutamine forming bidentate interactions with the N-Hand C═O groups on the backbone of a single residue (left) or consecutive residues (middle), or with the backbone N—H group and sidechain oxygen atom of a serine or threonine (right). Second row: as illustrated for the glutamine-backbone bidentate interaction case, to build the hash table we carry out Monte Carlo sampling over the rigid body orientation between the terminal amide group and the backbone, and the backbone torsions phi and psi, saving configurations with low energy bidentate hydrogen bonds. For each configuration, the possible placements for the backbone of the glutamine are enumerated by growing sidechain rotamers back from the terminal amide. Third row: from the six rigid body degrees of freedom relating the backbones of the two residues, and the phi and psi torsion angles, a hash key is calculated using an 8 dimensional hashing scheme. The hash key is then added to the bash table with the sidechain name and torsions as the value. (C) To dock repeat proteins and repeat peptides with compatible superhelical parameters, their superhelical axes are first aligned, and the repeat peptide is then rotated around and slid along this axis. For each of these docks, for each pair of repeat protein-peptide residues within a threshold distance, the hash key is calculated from the rigid body transform between backbones and the backbone torsions of the peptide residue, and the hash table interrogated. If the key is found in the hash table, side chains with the stored identities and torsion angles are installed in the docking interface. D) The sequence of the remainder of the interface is optimized using Rosetta™ for high affinity binding. Two representative designed binding complexes are shown to highlight the peptide binding groove and the shape complementary. The close-up snapshots illustrate hydrophobic interactions, salt bridges, and x-x stacks incorporated during design.

FIG. 2. Designed binders function in living cells. (A) Experimental design: U2OS cells coexpress the target peptide fused to GFP and a fusion between the specific binder fused to mScarlet™ and a mitochondrial targeting sequence (MitoTag™). If binding occurs in cells, the GFP signal is relocalized onto the mitochondria, while control cells not expressing the binder show cytosolic GFP signal. (B-E) In vivo binding. Live, spreading, U20S cell expressing PLPx6-GFP alone (B), IRPx6-GFP alone (D), PLPx6-GFP and Mito-RPB_PLP2 R6-mScarlet (C) or IRPx6-GFP and Mito-RPB_LRP2_R6_FW6-mScarlet (E) were imaged live by Spinning Disk Confocal Microscopy (SDCM). Note that the GFP signal is cytosolic in control but relocalized to mitochondria upon coexpression with the respective binder. F-G. In vivo multiplexing. F. Experimental design: cell coexpress two target peptides fused to GFP and mScarlet™ and their corresponding specific binder fused to mitochondria or peroxysome targetting sequences. If orthogonal binding occurs, GFP and mScarlet™ signals should not overlap. G. Live, spreading, U2OS coexpressing PLPx6-GFP, IRPx6-mScarlet, Mito-RPB_PLP2_R6 and PEX-RPB_LRP2_R6 FW6 imaged by SDCM. Note the absence of overlap between channels. Images correspond to maximum intensity z-projections (Δz=6 μm). Dash line: cell outline. Scale bars: 10 μm.

FIG. 3. Evaluation of design accuracy by X-ray crystallography. (A-C) Superposition of computational design models on experimentally determined crystal structures. (A) RPB_PEW3_R4-PAWx4, (B) RPB_PLP3_R6-PLPx6, (C) RPB_LRP2_R4-LRPx4, (D-G) RPB_PLP1_R6-PLPx6, (D) overview of superimposition of the computational design model and crystal structure. € 90 degree rotation of (D); the complex is shown in surface mode for shape complementarity, (F) Zoom-in interaction of the internal three-unit from (D) (front view); Glutamine residues from the protein in both design and crystal structure are as sticks to show the accuracy of the designed sidechain-to-backbone bidentate ladder. (G) Zoom-in interaction of the back view of (F); Tyrosine residues from the protein in both design and crystal structure are in sticks to show the accuracy of designed polar interactions on the other side.

FIG. 4. Design of binders specific for endogenous human proteins (A) Schematic model of the human PAXT complex composed of a heterotetramer of ZFC3H1 and MTR4. Domain acronyms: CC: Coiled-coil; ZN: Zn-finger domain, Inset shows the details of the environment of the target sequence (SEQ ID NO: 112). (B) Surface shape complementarity between the target peptide from ZFC3H1 (sphere) and the highest affinity cognate binder αZFC-high. (C) Fluorescence polarization binding curves between indicated ZFC3H1 binders and the target ZFC3H1 peptide (PLP) 4PEDPEQPPKPP (SEQ ID NO: 107). As a negative control, we used the (PLP)x6 binder, RPB_PLP3_R6 (see FIG. 3). αZFC-high shows higher binding affinity to the target peptide than αZFC-low, on the contrary to RPB_PLP3_R6, which shows negligible binding. (D) Superdex™ 200 10/300 GL size exclusion chromatography profiles of purified αZFC-high, a fusion between GFP and a 103AA fragment of the disordered region of ZFC3H1 containing the target sequence (see A.), or a 1:1 mix of the two after 2h incubation. (E) Top: Hela cell extracts were subjected to pull down using indicated binders bound to NiNTA agarose beads, or naked beads as control. Recovered proteins were processed for western blot against endogenous ZFC3H1 (or tubulin as a loading control), Bottom, coomassie stained, SDS-PAGE gel of the samples analyzed in top panel. These panels are representative of n=3 experiments. (F) Proteomics analysis of the his-pull down samples shown in C. Top panel: overlap between the proteins identified, setting s threshold of five peptides for correct identification. bottom panel: examples of proteins identified (number indicates exclusive peptide counts. protein coverage is indicated in parenthesis).

FIG. 5. Examples of repeat proteins computationally designed to bind to (A) extended beta strand, (B) polypeptide II, and (C) helical peptide backbones.

FIG. 6. Mitochondria immunostainings in control U2OS cells. Wild type U20S cells were spread onto fibronectin coverslips as in FIG. 2, then fixed and processed for immunofluorescence using TOM20 antibodies as a marker of mitochondria. Note that mitochondria appearance in these control cells is similar to that observed upon overexpression of designed binders fused to mitochondria targeting sequences (FIG. 2). suggesting that these constructs do not affect mitochondria shape. Scale bar: 10 μm.

FIG. 7. SSM binding interface footprinting results were consistent with the design model and crystal structure. (A) Using a PPL repeat peptide binder as an example, a heatmap presenting enrichment analysis for each mutation is generated. WT sequences are indicated in the cells labeled with amino acid one-letter codes. The mutants missing in the expression library are labeled with asterisks. Two positions (109Q and 156Q) are highlighted as examples showing conserved positions. Almost all mutations other than the WT in these two positions are greatly depleted. (B) Illustration shows the SSM region, and the two conserved positions (109Q and 156Q).

FIG. 8. Characterization of ZFC3H1 bindersHela cell extracts were subjected to pull down using indicated binders bound to NiNTA agarose beads, or naked beads as control. Recovered proteins were processed for western blot against endogenous ZFC3H1 (or tubulin as a loading control). Two completely independant experiments are shown. This experiments are repeats of the experiment presented in FIG. 4E, albeit at a different salt concentration, namely 50 mM instead of 150 mM.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In one aspect, the disclosure provides polypeptides comprising or consisting of an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 1-105.

The polypeptides of the disclosure bind tripeptide repeat sequences (for example, PLP, LRP, PEW, IYP, PRM and/or PKW) and thus can be used, for example as synthetic peptide ligands for receptor activation or to bind to proteins natively expressed by cells. The proteins are expressed at high levels in E. coli, are hyperstable, and bind peptides with 4-6 copies of the target tripeptide sequences with nanomolar to picomolar affinities both in vitro and in living cells.

The amino acid sequences of SEQ ID NO:1-105 are provided in Tables 1-3.

TABLE 1

| SEQ | order_ | | |
|---|---|---|---|
| ID NO | id | pdb_id | Sequence |
| 1 | IP_b1_01 | CP10_apo | PEEEAHKKASRRITQAHMYGKREEELEKILEEAIRELEKNGVDREEAAHRAASRLITQ |
| | | | AHMYGKDDRELEKILEIAIRILEKNGVDREEAAHRAASRLITQAHMYGKDDRELEKIL |
| | | | EIAIRILEKNGVDRREAAKRAASRLRTQAHMYGKHEEEEERIEEIAKRILEKNGHE |
| 2 | IP_b1_02 | CP6_apo | PEEEILVKAVNEASRITEKTKDEEKIKEALERILREAAERIRENGGNEEEAILVIAVN |
| | | | VASRITERVKNEEEIKKALEWILRVAAELIRENGGNEEEAILVIAVNVASRITERVKN |
| | | | EEEIKKALEWILRVAAELIRENGGNEEEAILVIAVNVASRITKRVTNEEEIKKAQHWI |
| | | | KHVAKKLIKENGGT |
| 3 | IP_b1_03 | CP_01_367_ | MEKEYIEKMKELYKKYREEGLSPEEAVQKAAEEVAKEIIEELKKEGKSPREAYREAMR |
| | | dldesign_ | VIVKFAEEQGLSKPEAVQIAAEAVAKIVVEELKKEGKSEREAAREAMRIIYEFAEEQG |
| | | 0_0 | LSKPEAVQIAARAVAKVVVEELRKEGKPEEEALEEAREIIREFARERGLSEEEAEQVA |
| | | | ERAVELVRRELEERDGGP |
| 4 | IP_b1_04 | CP_02_479_ | PKEEAEREATQRIHEALETMKSPEELKKVLEEAVKLLQEQGVPPPEAAKLAATQAIHY |
| | | dldesign_ | ALETMKSPEELKKVLKVAVEILKKQGVPPKEAAKLAATQAIHYALHTMKSPEELLEVL |
| | | 0_0 | EVSVELLKEQGVPPKEAAKLAATQAEHYAEKEKKSPEEVEEVKKVVEKLLKKQGVP |
| 5 | IP_b1_05 | CP_03_070_ | KPEEILREAVNEAYKFVLEEDDPEKIKEKLEKLLKEAAEKVKELGGDKEEAILKIAVN |
| | | dldesign_ | VATRVVLEEDDPEKIKEVLEIVLEVAAKIVKEYGGDKEEAILKIAVNVATRIVVESDD |
| | | 0_0 | PEKIKKLLKIVLEVAAKLVEKLGGDKVEAIKKIAKNVKELVKKELKDPEKIEEKSKII |
| | | | EEVEKELVKKLGGP |
| 6 | IP_b1_06 | CP_04_367 | PDEEAEKHIEEVVRRARENGSSEYMALQEAAFEVSRKIFEKIKRNGGDDREAAETAIE |
| | | | VVVRAARKGGSSEYMALQIAAFAVSRKIFEHIKRNGGDDREAAETAIEVVVRAARKGG |
| | | | SSEYMALQIAAFAVSRKIFEHIKRNGGDDREAAKTAKEVVVRAARKGGSSEYMAEQIA |
| | | | AFAVSRKEFEHRRRNGDS |
| 7 | IP_b1_07 | CP_04_367_ | MKEKYLEEMEKIYKEARKNGLSPEEAVQVAARKVAEKIIKEEKKKGKSEEEAYLEAMR |
| | | dldesign_ | IIYEFAKKQGFSPPEAVQIAAYAVAEIIIKDEEEKGKTPKEAYKKALKVIYKFAEEQG |
| | | 0_0 | FSPEEAVQIAAVAVASIIVKDIQEKGGSKEEAFETAYEIVKEFAEEQGFSPEEAEQIA |
| | | | KYAVEYNKKVEEKRNGGP |
| 8 | IP_b1_08 | CP_05_329_ | EEEEEIEREKKRAEKEGNEEAVDALENAEELYKKRPKDPVVKKLVKEAIRAAKEGNEE |
| | | dldesign_ | IVEALHNALVLYEKHPDDPVVKKLVEEAIRAAKEGNEEIVEALHNALVLYAKYPDDPK |
| | | 0_0 | VKKLVEEAIKAAKEGNEEEVKKIHNEIRKYAEDPSS |
| 9 | IP_b1_10 | CP_06_070_ | EEEKILQEAANEAYKHVKEETDKEKIKEKLRELLKTAAKKVKEQGGDEKEAILQIAAN |
| | | TtoV_ | VAYRVVKEETDKEKIKERLRIILKTAAELVEELGGDKKEAILQIAANVAYRVVKEEND |
| | | dldesign_ | EEKIKERLRLILSTAAELVEELGGDKKEAIETIAKNVIEHISKELKDPELIKKREEII |
| | | 0_0 | TEVAEELVKELGGE |
| 10 | IP_b1_11 | CP_07_131_ | PEKEKKEIEEEVKKKAEELKKKGKSLEEIVKELARYIVEVAEKAEKEGKPEIAKIVID |
| | | dldesign_ | VAVIVIAELLKSEGKSLEEIVKLLARFIVEVAEEAEKRGLPEVAKLVIDVAVIVIAEL |
| | | 0_0 | IKSEGKDLPEILKLMARFAVEVVEEARKRGLPEVAKLVLDVARIVAEELIKSEGLDPE |
| | | | EVEEKMEEFEKLVEELLKERGI |
| 11 | IP_b1_12 | CP_08_479_ | PVEEAREEANKEITEALEKGKSPEELKEVMRRARERLEKAGVPPKEAAIIVANIAITY |
| | | dldesign_ | ALEKGKSPEELKEVLKVAVEILEEQGVPPKEAAIIAANIAITYALEKGKSPEELRKVM |
| | | 0_0 | EVAVEVLEEQGVPPKEAAKIAANIAETRAEEIGKSPEELEEVRKIAEEILKKQGVP |
| 12 | IP_b1_13 | CP_09_ | PKEEAEKKTTERLHKTLKNRKSPEELLKAMEEAVKELKEQGVPPPEAAKIATQQLLAE |
| | | StoV_LtoK_ | ALKERKSPEELLEVMKVAVEVLKKQGVPPPEAAKIATQQLLAEALRLRKSPEELLEVM |
| | | dldesign_ | KVAVEVLKEAGVPPPEAAKIAAQQLLHEARKHEKSPEELEEVEKVVEELLKEQGIP |
| | | 0_0 | |
| 13 | IP_b1_17 | CP_12_114_ | PELEKTKKEVEKALKEAKKEGNEETAEAAEEALEALDDAEVSNNPEAVETVTKVAKLL |
| | | dldesign_ | LKAIKEGNEETAKAALEALKALHDAIVSKNPEAVETVTKVAELLLKAIKEGNEETAKA |
| | | 0_0 | ALEALKALHDAIVSKNPEAVEEVTKVAEKLLKAVKEGNEEEAKKALEKLKELHDRIVS |
| | | | KE |
| 14 | IP_b1_18 | CP_13_366_ | DLKKKLEENIEHLEKLAEKRGDPRLKEVVKEAKKAIESGDPEQLKLAHNAIVLAIEAV |
| | | dldesign_ | ERGDPRLEEVVKEALKALKSGDPHQLKLAHNAIVLAIEAVERGDPELLEVVKVALKAL |
| | | 0_0 | KSGDPEQLRKAHNAIVLYREAVEKGDPELEKEVKELLEEVKKGE |
| 15 | IP_b1_20 | CP_15_548 | PREEAWLTADSASIKGSKEDIEEATRRITKTNHKRQAAWLLADSASIKGSKEIIEEAT |
| | | | RQITETNHKRQAAWLLADSASIKGSKEIIEEATRQITETNHKRQAAWLLADSASIKGN |
| | | | EEIIREAERQIKKTNE |
| 16 | IP_b1_21 | CP_15_548_ | PREEAYKLISKAVEKGDPEEIEKAFKEAEKKLSPREAANLLISEAVKKGDPELIERAF |
| | | dldesign_ | ELAEKALSPREAANILISEAVKKGDPELIELAFELAHKALGPEEAARILLSEAREKGD |
| | | 0_0 | PELIKLAEELAKKARP |
| 17 | IP_b1_22 | CP_16_662_ | PEEELERRVSQIAVEAVEAKAKGDEKKVKELIEEVKELAKELGLSPEKAFKVFVAQIA |
| | | dldesign_ | VLAVEAQEKGDEKKVKAALEVVKEAAEVLGISPEKAFKVFVAQIAVDAVEYAAKGNEK |
| | | 0_0 | KVEAALRVAKLAAEVFGISPEKAYKVFSYQIEKLKEEYKKEGNEKKVEAADKVIKKLK |
| | | | EVLGLP |
| 18 | IP_b1_23 | CP_17_671_ | PKLEENEKALEEKKKELEKIAKEGTPEDKKMAKKATELAEKLVKIAEEEKDPELLKDL |
| | | dldesign_ | VTLAVKALELAKLAAHGTPEAKKMATKALELAEKLVKIAVEEKDPELLKDLVTLAVKA |
| | | 0_0 | LELAKLAAHGTPEAKKMATKAMELAEKLVKYAEEQKDPELLKNLVVKAVKLLELAKKA |
| | | | ADGTPEEKKEAEKEMKKIEEELKKKEENNE |
| 19 | IP_b1_24 | CP_18_062_ | ERYEKVKEEIEKKAKEDPNNKPVKNALVLIKEAEKAKKKGLEEWAELALKIAEELLKN |
| | | dldesign_ | PENKPVDNAAILILEAIKAKEKGLEELAKVALEIAKELLENPENKAVDNAAILILEAI |
| | | 0_0 | KAKEKGLEELAKEALETAKELLENPENKETDNKAVELIEKIRKEKKGE |
| 20 | IP_b1_25 | CP_19_315 | PEEEIFYRASTATQELERRGRDEEEAVEEAAREVIKHIEGRETAIFYAASTATQSLER |
| | | | NGRDEEEAVENAARAVIRTIEGRETAIFYAASTATQSLERNGRDEEEAVENAARAVIR |
| | | | TIEGREEAIFYAASTATQSLERNGRSEEEARETAKRAVDRTIET |
| 21 | IP_b1_26 | CP_19_315_ | DEEEILRDANERIQKRLKEGLSPEEAIEEATREIAEKYPNKELAVFLAANAAIQILLR |
| | | dldesign_ | EGLSPEEAIEIAAKAVAKAVPNKELAVFLAANAAIQILLREGLSPLEAIEIAAKAVAK |
| | | 0_0 | AVPDKELAKKLTHNAAVQILKRRGLSPEEAEKIAEEAIKKAFPE |
| 22 | IP_b1_27 | CP_20_336_ | MEEEVRKAVEEALNTYLKIKKEGGSEEEAVKEAAKIIVETVKKAGGTPEDIVRAAVAL |
| | | dldesign_ | ALNVYLKVRKEGGSVEEAVELAAKVLVVTVKEAGGTPEDIVRAAVALALNVYLKVEKE |
| | | 0_0 | GGSPEEAVRLAALVLVTTVKLAGGTPEDIVRAAELLSENLERRVKEEGGSPEEAERRR |
| | | | ELVLKTTKELAGLP |
| 23 | IP_b1_32 | CP_25_367 | PDEEAEKHIEEVVRRARENGSSEYEALFEASQEVASKIFEKIKRNGGDDREAAETAIE |
| | | | VVVRAARKGGSSEYEALFEASQAVASKIFEHIKRNGGDDREAAETAIEVVVRAARKGG |
| | | | SSEYEALFEASQAVASKIFEHIKRNGGDDREAAKTAKEVVVRAARKGGSSEYEAEFEA |
| | | | SQAVKSKEEEHRRRNGDS |
| 24 | IP_b1_33 | CP_25_367_ | MREKYLEKMKKIYEEAEKKGLSPREAVHIAAQKVAEEVIRELKEEGKSEREAYLEALR |
| | | dldesign_ | IIVEFAKEQGLSPREAIHIAAQAVAKLVIRELKEEGKSRREAYLRAMDIVYEFAREQG |
| | | 0_0 | LSPEEAVHIASRAVAKLIIRELEEEGKSPEEATETARRIIEEFAESKGLSPEQARRIA |
| | | | EQTVERIVEQDRKRKGSP |
| 25 | IP_b1_36 | CP_27_474_ | PEEEKLAKEQAEHLYDDPEEREVAEKVIIEAIKRVKEMGLPPEVALKAATQAVSLFKE |
| | | dldesign_ | LEEARVAAKVILEAIKRVKEMGLPPEVALKAATQAVSLFKELEEAKVAAEVILEALKL |
| | | 0_0 | VKEMGLPPEDALRAATQAVSKHKELEPAKEEARRLLEEILRNRKKGEP |
| 26 | IP_b1_37 | CP_28_479_ | PVEEAIEKAEKEISEAVENKKSPEELKEVLRKAVKKLEEQGVPPKEAAVRAARLAIDV |
| | | dldesign_ | AVDVKKSPEELKEVLKVAVEILEEKGVPPKEAAVIAARLLIDVAVDVKKSPEELREAL |
| | | 0_0 | EVAVEILEEQGVPPKEAARIASELLLDLVRDHSKSPEEEREAEEVAREVRKKRGVP |
| 27 | IP_b1_38 | CP_29_548_ | PKPRAHELLDEAVKSGDPEEIERAVREVTRLLPPREAAHLLLDVAVQEGDPELIERAV |
| | | dldesign_ | RLVTRLLSPREAAHLLVDVAVQEGDPELIERAWRDVTRLLSPEEAARILKDVAEQEGD |
| | | 0_0 | PELLERARELPKEAEP |
| 28 | IP_b1_39 | CP_30_638_ | PEPEIKKLIEEAKKKAKEEADKIVKEKKKKKAPEEEIVKEVVESLVRKLQEVLEKLVE |
| | | dldesign_ | ILKENGVPPEEIIERVKEVAHELAKEAAKILVEVLSKHGVPRELVVKAVVLFLVLALE |
| | | 0_0 | SLLRLLVELLKEQGVPPEEILREVRETALELAKEAAKIVVEVLSKHGVPEELVVKAVV |
| | | | EFLVLALESLLRLLVELLKEQGVPPEEILERVREEAERLKEEALKIVKEVLSKHGVPP |
| | | | EDVEKAVERLKRLVEQSLERLLEEIRREQGHP |
| 29 | IP_b1_40 | CP_31_638 | PEEEEERHKEEAVKEAEKKAKHAVKEAKKNNEDDDEILHKLVLLLSLIIQIILLELEK |
| | | | ILEKNGTSREEIEETIKEVAVKLAEEAAKIAVKVAKENNQEDDKILHALVLLLSLIIQ |
| | | | IILLTLEKILEKNGTSREEIEETIKEVAVKLAEEAAKIAVKVAKENNQEDDKILHALV |
| | | | LLLSLIIQIILLTLEKILEKNGTSREEIEETIKKVAKELAHEASRIAEKVAKENNQED |
| | | | EKIKHALELLQSLIEQIIKLTEEKIKRKNGHE |
| 30 | IP_b1_41 | CP_31_638_ | PEEEIKEEVKKAREEAKKEAEKIVKTLKEKGVPEEEVVEAVVEHLVQKIKELLEKLVE |
| | | dldesign_ | MMKKNGVPPEEIKKEILKTAKELAKKAAEIIVEVLKKHKVPEELVVEAVVKYLVLAIE |
| | | 0_0 | VLLRILKELLKEMGVPPEEIIKEIKETAKELAKKAAEIVVEVLKKHKVPEELVVEAVV |
| | | | KYLVLAIEVALRILEELLKEMGVPPEEIIKKIEETAKELLEKAEKIVREVLKKNNVPP |
| | | | ELVEQAVEEFKKLAEQMLKQILERLRKENGHP |
| 31 | IP_b1_42 | CP_32_638_ | MEEEIEKHVKEFKEKAKEEAKKIVETLKKEKKPEEEIVKEVVEYLVRKLQEALEELIE |
| | | dldesign_ | LLKEQGVPPEEILKRVLELAKELAKELAKIIVEVLKEFGIPEEEIVKAVVEFLVLALQ |
| | | 0_0 | VALELVKEILKEQGVPKEEILKKVLELSKELAKELAKIIVEVLKEFGVPEEEIVKAVV |
| | | | EFLVLAVQVALELLKEILKEQGVPLEEIKKKVEEYAKELAKELEKIIKEVLKEFGVPE |
| | | | EEIEKAVKEFKKLFEQKLKELLEEIRKEQGEP |
| 32 | IP_b1_53 | CP_PX_01_ | PEEKELEESAKENEEEAKKYEEKAKELEKKGKKEEADFLLTLAAALKVAAQAYKAIAE |
| | | 728_ | AEKKKDDPSLELYEEAAELAKEAAELVKKAEEKYKKGKKEEADYYLLEAAALIVAAQA |
| | | dldesign_ | LLAIAEARKKKDPDLEELSEEALELATKAAELVKKAEELWKKGKKEEADFYLLEAAAL |
| | | 0_0 | IVAAQALLAIYEAKKKKDKPALEKAEKAAELAKEAAKLVKEAYKLYKKGKKEEAKKYK |
| | | | EEAKKLIKEAQELLKERVLEKNNE |
| 33 | IP_b1_54 | CP_PX_02_ | PREEELEETAHRLEKRSREAKKRAEELRRNGDEESATLLELLATALSTAAQAASMRSH |
| | | 728 | AEQNNGERELRLAETAYRLAKRSSEAVERAEELRRNGDEESATLLELLATALSTAAQA |
| | | | ASMRSHAEQNNGERELRLAETAYRLAKRSSEAVERAEELRRNGDEESATLLELLATAL |
| | | | STAAQAASMRSHAEQNNGERELRLAEKAYRLAKESSEAVEKAEELRRNGDEDSATKLE |
| | | | LRATELSTKAQRASMESHREQNNE |
| 34 | IP_b1_55 | CP_PX_02_ | PEKKHLETRAKKYKEEAEKLRKKAKELKKKGKEKEAHFYKTKAAALTTAAQALEKIYE |
| | | 728_ | ARKKKDPELEELSHEAARLATEAAELVEKAEKLAKKGKEKEAEYLKLKAAALTVAAQA |
| | | dldesign_ | LLKIYEARKKKDKPAEELSKKAFEYAKEAAELVEKAKELEKKGDKKEAEYYKLKAKAL |
| | | 0_0 | TVAAQALLAISEARKEKDPELEKLSTEAFELAKEAAKLVKEALKEEKKGDKKKATELK |
| | | | KKAKELIKKAQEKLEEYYKKKENL |

TABLE 2

| SEQ | | | |
|---|---|---|---|
| ID NO | order_id | pdb_id | Sequence |
| 35 | IP_b1_83 | PXX_001_ | PELEKLRKKVEELAKKAKEDGDEEAAKKFKELLNAVEAAEKAEKEGLKEIAELTVKVA |
| | | X_007_ | KKAIEAEENKDEEAAKKYLELANVLVAAYYAEREGLKEIAELTVKVVTEFLKAAERKD |
| | | dldesign_ | EEAAKKYLELANVLVAAYYAEEEGLTELAKKTVEVVKKFLKAAQNKDEKEAKKYLDEA |
| | | 0_0 | NKLLKQYHDHENGK |
| 36 | IP_b1_84 | PXX_002_ | PEVERLRKEVKEEADKARENNDEDSLRKWKLLANALIAAHFAHLNGLEEVARLATEVV |
| | | StoD_ | HEAYEASRNNDEDSLRKWLLLANALIAAHFAHLNGLEEVARLATEVVHEAYEASRNND |
| | | LtoW_ | EDSLRKWLLLANALIAAHFAHLNGLEEVAHEATRVVHEATRASEENDEESLEKWLLEA |
| | | scored | NKLIATHFAHLNGE |
| 37 | IP_b1_87 | PXX_002_ | GEIKELKEKVTKKAEEAKKNGDEEEAQKFEELLNALEAAEKAEKEGNKEIADLTVKVV |
| | | X_007_ | KKALEAYEKGDEEKFQKFLELANALVAAYYALKEGNKEIAELTVKVVKKAVEAYEKGD |
| | | dldesign_ | EEKFQKFLELANALVAAYFAYKRGNKELAEKLVKYVKKFLEAEEKGDEEKAQKYLDKA |
| | | 0_0 | NELVKEYEDLEKGK |
| 38 | IP_b1_95 | PXX_010_ | PESEKIEKLAREWEKRGGKEEAFIASVIAQLVEAAERRGSEELLKIAELAAEWLERGG |
| | | X_410 | REEAFIASVIAQLVEAAERRGSEELLKIAELAAEWLERGGREEAFIASVIAQLVEAAE |
| | | | RRGSEELLRIAEEAAEWTERGGREEAFHASVRAQLVEAAERKGT |
| 39 | IP_b1_96 | PXX_010_ | PVAEEVEKLAEEFKKRGDEEEAELARRAAELIRRAEERGNPELKEVAELAAKALKKGD |
| | | X_410_ | VEEAKLALRAAELIERAEERGNPELKEAALLAAKALKKGDVEEAKLALRAAELIERAE |
| | | dldesign_ | EAGNPELLKIAKEAAEALKKGKVEEAKKLIKEAEELKEKHEKGE |
| | | 0_0 | |
| 40 | IP_b1_97 | PXX_011_ | EPLEKVKELAKKYEKEGNKEKAELAKIIAKLIKEAEKEGNEELLEVAKLAAKYLEEGK |
| | | X_410_ | FEKAKLALIAAKLIKEAEKEGNEELLEAAKLAAKYLKEGKEEKAKLALIAAKLIKEAE |
| | | dldesign_ | KEGNEELKKTAKKAVEAIKKGKEEEAKKLLKEAEKIKKKREKGE |
| | | 0_0 | |
| 41 | IP_b1_98 | PXX_012_ | EVEKAKKKAEKIITEALKNGKSPEELRKAMEKAVKELKKAGVPPKEAARIAAQIAITY |
| | | X_479_ | ALKDGKSPEELEEVMRIAVEVLRKQGVPPKEAARIAAQIAITYALKHGKSPEELLKVM |
| | | dldesign_ | KIAVKILREQGVPPKEAARIAAQIALYYAKKHGKSEEEIKEVEKIPEEVLKEQGVP |
| | | 0_0 | |
| 42 | IP_b1_99 | PXX_013_ | PEEEAFLKASIRIQHAHEHGKREEELEKILEEAIRELEKNGVDREEAAFLAASILIQH |
| | | X_479 | AHEQGKDDRELEKILEIAIRILEKNGVDREEAAFLAASILIQHAHEQGKDDRELEKIL |
| | | | EIAIRILEKNGVDRREAAFLAASILIQHAREQGKHEEEEERIEEIAKRILEKNGHE |
| 43 | IP_b1_100 | PXX_013_ | PEQEAFDEAAERIEEALRNGKSPEELREAMREAVKRLQEQGVPPKEAAFQAAAILIEE |
| | | X_479_ | ALEEGKSPEELREALEVAVEILKEQGVPPKEAAFQAAAILIEEALEEGKSPEELREAL |
| | | dldesign_ | RIAVELLQKQGVPPKEAAKLAPKIAEQEAEKEGKSPEEIERVKKVAEEILKEQGVP |
| | | 0_0 | |
| 44 | IP_b1_101 | PXX_014_ | PKEEAIEKAREKINELLENSQSPEELKEAMKEAVKLLQKQGVPPKEAAKIAAEILINA |
| | | X_479_ | ALESSQSPEELKKAMKIAVEVLKEQGVPPKEAAKIAAEILINAALESSQSPEELKTVL |
| | | dldesign_ | EIAAEILKEQGVPPKEAAKIAAEILENVAKESSKSPEEEEEVEKVVKEILKKQGIP |
| | | 0_0 | |
| 45 | IP_b1_102 | PXX_015_ | PEEEAEKKAKEEITQTLANGKSPEELKEVMEKAVKELQKQGVPPKEAAKIAAKIAITQ |
| | | X_479_ | ALAHGKSPEELKEVMEIAVEILKKQGVPPKEAAKIAAKIAITQALAHGKSPEQLREVM |
| | | dldesign_ | ELAIEILKKEGVPPKEAAKIAAKIAEQQAKAHGKSPEQIKEVEKVAKEILKKEGIP |
| | | 0_0 | |
| 46 | IP_b1_103 | PXX_016_ | NKEEAEKLLKELEKKGVPPEEAAITAAYELAEKLLREGKPLEEAAEAALLALKLAGVP |
| | | X_518_ | PEEAAILAAYVVAEALLRLGRPEEDAAKSALLALKRAGVPPEEAARLAAYVIAEALLR |
| | | dldesign_ | LGRSEEDAEKTALLALKLAGVPEEEAEKLVKKVLEQVKKRLGAP |
| | | 0_0 | |
| 47 | IP_b1_104 | PXX_017_ | LEERAKERLEELKKKGVPPEEAARRVATELVAELKEEGKSLEEIAETALLAVKLAGVP |
| | | X_518_ | PEEAARIVATQIIAVLKQLGESEEEIAETALLALKKAGVPPEEAARIVATQIIAVLLQ |
| | | dldesign_ | LGESEEEAIEAAKLALKRAGVPEEEAEKLVEKQKEAVRKQAGEP |
| | | 0_0 | |
| 48 | IP_b1_105 | PXX_018_ | PEEEEVRHAIERAEEIAKEAKEKGLEELAELAKRVAEELKRAIEEGDKEKIEALKALV |
| | | X_534_ | VALQIALEAHKKGLEELAKLAVEVVRELLEAIREGDKEKIEALKALVVALQIALEAHK |
| | | dldesign_ | KGLEELAEAALEVLKLALEAIRKGDKEKLERLKELVRDLQKALEADKKGKKEEAEEHV |
| | | 0_0 | KRVHEEIERIKKGE |
| 49 | IP_b1_106 | PXX_019_ | PERERVERAIERARQIAEEAKKQGNEELARTAEEVARLMEKALKEGDEKTLKYLLNAV |
| | | X_534_ | VALQIALEAKKEGNEELARTATEVAKLIVEAIKKGDEKTLKYLENAVVALQIALEAKK |
| | | dldesign_ | EGNEELAKLALEVAKLLVEAIKKGDEETLKKLEEAVVLLQKALEAKKEGKEEEAKKAL |
| | | 0_0 | KEAKELVEKIKKGE |
| 50 | IP_b1_107 | PXX_020_ | PEIEKVREEAEKLLKEAKKKKDEKTIKKLEKVLKAARIAEIAKQQGLEEVAKIAAEAA |
| | | X_540_ | KLALEAIKKKDEKTLKKLEKVLKAARIALIAHQQGLEEIAEVAKKAAKEALKAIKKKD |
| | | dldesign_ | EKTLKKLKKVLKAAEIALIAYQNGLEEIAKKALEAAKKALEAIKKKDEKTLKELKKEI |
| | | 0_0 | EKAEKELRDADKGK |
| 51 | IP_b1_108 | PXX_021_ | PEIEKIKEEAEKLKKEAEKEKDEKKLKNLKEVKEAAKIAETAKQLGLEEAAKIAAEAA |
| | | X_540_ | KLALKAVKEKDEKKLKNLKEVIEAAKIAATAYQLGLEEIAKAAIEAAKEALKAIKKKD |
| | | dldesign_ | EKKLKNLKEVIEAAKIAATAYQNGLEEIAERAAKAAKELLKAIKKKDKKKLKELKKEI |
| | | 0_0 | EEAKEELRKAQEGK |
| 52 | IP_b1_109 | PXX_022_ | PEEEKLKKLAENAKKVAKEAKKEGDKELAELSKKAVEALEEAIKKGDEETIRAARKAL |
| | | X_628_ | EAAVVAYHAKKEGDKELAELSLKAVKLLLEAREKGDEETIRAAEKALEAAVVAYYAKK |
| | | dldesign_ | EGDKELAELSLKAVKLLLEAREKGDEETIRKAEKLLELAVKAYHAKREGKEEEYKELL |
| | | 0_0 | EKVREEIEKHEKGE |
| 53 | IP_b1_110 | PXX_023_ | PEVEKAKKELEKEIKEAKEKGDKELVEQAKLAKEALENAEKSGDPELEKAALELVKLL |
| | | X_632_ | KKAEKKKDEEAVEQIKLAAEALINATYSGDPELIKAALELVKELKKAEKKKDEEAVEQ |
| | | dldesign_ | IKLLAEALINATYSGDPELIKKALELLKRLEKAEKKKDEKEVEKVKKEAKELINKTYE |
| | | 0_0 | NE |
| 54 | IP_b1_111 | PXX_024_ | GPVEEAKKKLEELIKEAKEKGDEEKVRQLQNAKEALESAEESGDPEAEKASEELVKDQ |
| | | X_632_ | IEAIKKGDEEKVRQVQEAIEALISALYSGDPEAIKASLELVKDQREAHKKGDEEKVRQ |
| | | dldesign_ | VQEAIEALISALHSGDPEAIKKSLKLYEEQLEAYKKGDEEELKKVSEEIRELIDKLHL |
| | | 0_0 | NE |
| 55 | IP_b1_114 | PXX_027_ | PERERVEFAARYAHQEAKKKLEEAKRHGNEEETEEWKRETELVRRLAELAHKSDREEV |
| | | X_690 | LRVIFAALYALQEANKKLDEAKRHGNEEETEEWRKETELVKRLAELASKSDREEVLRV |
| | | | IFAALYALQEANKKLDEAKRHGNEEETEEWRKETELVKRLAELASKSDREEVLRTIFA |
| | | | ALYNLQEANKKLDEAKRHGNEEETEHWRKRTETVKREAEKASKSDK |
| 56 | IP_b1_115 | PXX_027_ | EPKERVKEAAEYLLKYFKEELKKAKKKGDKKEAEHYEKLIKEVEKAKKYADEATDPEK |
| | | X_690_ | LDVIAAALTLLEKFTKLYKEAKKKGDKEEAEKYEKLIEAVRKALEYALEATDPEKLDV |
| | | dldesign_ | IAAALTLLEKFTKLYKEAKKKGDKEEAEHYEKIIDAVLKALEYATEATDEEKLKIIAE |
| | | 0_0 | KLKELEKLTKEYKEAKKKGDKKKAKEYKEKIEKTLKELEEAKKRKK |
| 57 | IP_b1_116 | PXX_028_ | PEEERKKEAEEVAAHVEQIAFIAKEQGNEEVAKLAKRLAETIKRLNEGTEEEVKRLLE |
| | | X_719 | AAEVAAHVLQIAFIAHEQGNEEVAKLALELAESILRLIEGTEEEVKRLLEAAEVAAHV |
| | | | LQIAFIAHEQGNEEVAKLALELAESILRLIEGTEEEVKELLERAEEAAHVLQHAFIAT |
| | | | EQGNEEDAKEALRKAEEILRRNEDRR |
| 58 | IP_b1_117 | PXX_028_ | PREEKKKKAEEAAEYVEQIAKEAKKEGNEEIAKVAEELAKEIRKLVKSEDPEDEKKLE |
| | | X_719_ | ALVAAAYVLQIAYEAKQEGNEEIAKLALELAKEILKLVKSEDPEDEKKLEALVAAAYV |
| | | dldesign_ | LQIAYEAKREGNEEIAELALELAKEILKLVKSEDPEDEKKLEELVKAAKVLQKAYELK |
| | | 0_0 | KEGKEEEAKKYLEEAKKILEKVKSKE |
| 59 | IP_b1_118 | PXX_029_ | PEELIKEAKKVLEEIEKKYKQDPNNEALKLAYKAAHNAYVAAKEAKETPEPFKELLKE |
| | | 016_ | AVKVTLEIAKKLAEDPENEALKLAVIASYNTLHAAKAAEETPEPFKELLKEAVKVTLE |
| | | dldesign_ | IAKKLAEKPENEALKLAVIASYNTLHAAKNAEEAPPPFKEKLSEAVKETLKYAKKLAE |
| | | 0_0 | KPEDEELKKKVEETYNKLLELEKEETTPPP |
| 60 | IP_b1_129 | PXX_040_ | PEHVIKRAVMEAVNRAETEKRNGGDEEEALRLAAEVVIHVVEEEGGSEEWVIKAAVMV |
| | | 336 | AVNLAETEKRNGGDKEEALRLAAEVVINVVENSGGSEEWVIKAAVMVAVNLAETEKRN |
| | | | GGDKEEALRLAAEVVINVVENSGGSEEWVEKAAVMVAVNLAETEKRNGGDEEEAERLA |
| | | | EEVRRNVVERSGGS |
| 61 | IP_b1_130 | PXX_040_ | PEEKIREAAQEALNRANEIEKEGGSPEEAIEVAAEILIERVKKAGGTEEDIVDAAVRV |
| | | 336_ | AVNLANKIRQNGGSREEAIKLAAEVLIEVVKKAGGTEEDIVDAAVRVAVNLANKILQN |
| | | dldesign_ | GGSREEAIESAALVVIITVKRAGGTEEDIRDAAERVAKNLKEKIKENGGSPEEAEKYA |
| | | 0_0 | KEVRETTKKRAGLP |
| 62 | IP_b1_131 | PXX_041_ | DKEEYLEEMKKIYEEARKEGKTPEEAVLTAAKRVANKIFVDLRKQGKSEEEAAKKAME |
| | | 367_ | IIYEFAKEQGLSPEEAVKIAAKAVANVVFVHLREQGKSIKEAAKEAARIIVEFAKEQG |
| | | dldesign_ | LSPEEAVKIAAKAVANIVFVYKRDEGKSEEEAKKEAKEIIYELAEEEGLSEEEAKKIA |
| | | 0_0 | EEAVEEVVKRFRKSRGEP |
| 63 | IP_b1_132 | PXX_042_ | EKFEKEKKELKKEAEKLKKEAKKAKKEGNKEEAKALEEAVEALELAVDVIDNAEEKND |
| | | 435_ | PEAAEEAAKLAKEAVELAEKAVKAIKEGNKEEAKALELAVEALELALWVIYNAVIKND |
| | | dldesign_ | PEAAEEAAKLAKEAVELAKKAVKAIKEGNKEEAKALELAVEALELALWVIYNAVIKND |
| | | 0_0 | PEAAEKAAKLAEEAVEYAKKAVKAIKQGDEKKAKKLKEKVKKLLEELKKLYNELLEGK |
| 64 | IP_b1_133 | PXX_043_ | PEEEKRTKHLEEVAETVRERAKEVIDKDNDEEVKNLATWAEEAAHTVEEALKTARNDT |
| | | 474 | GKEEKERLDHLVEVASTVIERAIEVIEKDNDKEVLNLATWAIEAAVTVLEALRTARND |
| | | | TGKEEKERLDHLVEVASTVIERAIEVIEKDNDKEVLNLATWAIEAAVTVLEALRTARN |
| | | | DTGKEEKERLDHLVEEASRVIEEAIKEIEKDNDKRVLNLATWKIEKAVEELKRLRTER |
| | | | NDKN |
| 65 | IP_b1_134 | PXX_043_ | EEKEKKEKELEKLVKKVEELMKKLLEKDPTPENKILEKIFVESAKEVLKAYKKALSSP |
| | | 474_ | DEEAKKKLEELVEVAKEVVEKALELLKQDPNEEVLLLSTILVASAVEVLKALKHALSL |
| | | dldesign_ | PDEEKKKKLEKLVEVAKEVVELALELLKQDPNEEVLLLSTILVASAVEVLKALKHALS |
| | | 0_0 | LPDEEKKKELEKLVEEAKKVVEEALKKLKENPNEETLKEAVIKTSEAVEKLKKKIKEI |
| | | | ESEE |
| 66 | IP_b1_135 | PXX_044_ | PEEKAEEEAKKMITETYQNGKSPEELKKAMKEAVKELQKQGVPPKEAAKIAAEIAITT |
| | | 479_ | AYQDGKSPEELKKVLELAKEILEEAGVPPKEAAKIAAEIAITTAYQDGKSPEELKKVL |
| | | dldesign_ | KVAVELLKKTGVPPKEAAKIAAEIARRVAKQDGKSPEEEKEVEKVVKEILEEEGIP |
| | | 0_0 | |
| 67 | IP_b1_136 | PXX_045_ | PEEEAYRKAQKRIHTAHEHGKREEELEKILEEAIRELEKNGVDREEAAYRAAQILIHT |
| | | 479 | AHEQGKDDRELEKILEIAIRILEKNGVDREEAAYRAAQILIHTAHEQGKDDRELEKIL |
| | | | EIAIRILEKNGVDRREAAYRAAQILRHTAREQGKHEEEEERIEEIAKRILEKNGHE |
| 68 | IP_b1_137 | PXX_045_ | PKEKAYKEAQDRIVEALKKGKSPEELKEELKKAVERLKKQGVPPKEAAEIAAETAIRV |
| | | 479_ | ALEEGKSPEELKEVLEVAVEILEEQGVPPKEAAEIAAETAIRVALLEGKSPEELRKVL |
| | | dldesign_ | KVAVEVLEKQGVPPKEAAEIAADTALEYAKKEGKSPEEEEEVEKVVKEIKKEKGVP |
| | | 0_0 | |
| 69 | IP_b1_141 | PXX_049_ | PEREKVEKLARNAHLWAKKKLEEAKRHGNEEETEEWKRETELVRRLAELAHKSDREEV |
| | | 690 | LKVIKLALNALLWANKKLDEAKRHGNEEETEEWRKETELVKRLAELASKSDREEVLKV |
| | | | IKLALNALLWANKKLDEAKRHGNEEETEEWRKETELVKRLAELASKSDREEVLKTIKL |
| | | | ALNNLLWANKKLDEAKRHGNEEETEHWRKRTETVKREAEKASKSDK |
| 70 | IP_b1_142 | PXX_049_ | ELKEKVKELAENLLEILKKKLKEAKEKGDKKEAKFYEELIKAVEKAKEYAEKATSPVQ |
| | | 690_ | LKVIALALNLLQHLIEAYEEAKKKGDKEEAKEYEELLKAVLKALKAASKATSPVQLKV |
| | | dldesign_ | IALALNLLQILIELLKEAKKKGDKEEAKHYEEILKAVLKALKYAHKATSPVQLKKIAK |
| | | 0_0 | ALNELQILIEELKKAKKKGDKKKAKEKKEEIKKVLKELKEAKKLKK |
| 71 | IP_b1_143 | PXX_050_ | KPEEVRERATQTIRELLEKGKSPEEAIEEATKEVLKEVPDKELAVRETAAQAIQILLS |
| | | 315_ | QGHSPEEAIEIATKAVLEAVPDKELAVRETAAQAIQILLSQGHSPEEAIEITTKAVLE |
| | | dldesign_ | AVPDKELARRETRRQAIQILRSRGLSPEEAEEITEKAVEAAFPE |
| | | 0_0 | |
| 72 | IP_b1_144 | PXX_051_ | DEQEIEEAATQLIAERLKQGLSPEEAIKTTAKELAEKFPDKELVLELVATQAIAVLLQ |
| | | 315_ | QGLSPEEAIKITAKALAEAFPDKELVLRLVATQAIAVLLQEGLSPKEAIKITAKALAE |
| | | dldesign_ | AFPDKELVKKLVHKQAIHVLKQQGLSPEEAEKIAEEAIKEAFPE |
| | | 0_0 | |
| 73 | IP_b1_151 | R6PEP12 | DEEREKLKEKLKEVLRRAKEAKKKGDKEKLIELAYEAAALAAWIIHKDSNDDEIVELA |
| | | | KEALKLVLEAAKEAKKNGDKEKLIKLAYLAAAVAAWIIHTDGDDDEIVELAKEALKLV |
| | | | LEAAKEAKKNGDKEKLIKLAYLAAAVAAWIITTDGDDDEIVELAKEALKLVLEAAKEA |
| | | | KKNGDKEKLIKLAYLAAAVAAWIIHTDGDDDEIVELAKEALKLVLEAAKEAKKNGDKE |
| | | | KLIKLAYLAAAVAAWIITTDGDDEEIVELAKEALKLVKEAAEEAEKQGDEELREKLRY |
| | | | LSEAVREWIERNDGDD |
| 74 | IP_b1_152 | R6_02 | PEEEILVKAVNEASRITEKTKDEEKIKEALERILREAAERIRENGGNEEEAILVIAVN |
| | | | VASRITERVKNEEEIKKALEWILRVAAELIRENGGNEEEAILVIAVNVASRITERVKN |
| | | | EEEIKKALEWILRVAAELIRENGGNEEEAILVIAVNVASRITERVKNEEEIKKALEWI |
| | | | LRVAAELIRENGGNEEEAILVIAVNVASRITERVKNEEEIKKALEWILRVAAELIREN |
| | | | GGNEEEAILVIAVNVASRITKRVTNEEEIKKAQHWIKHVAKKLIKENGGT |
| 75 | IP_b1_153 | R6_16 | PESEKIEKLAREWEKRGGKEEAYIASVIAQLVEAAERRGSEELLKIAELAAEWLERGG |
| | | | REEAYIAVVIAQLVEAAERRGSEELLKIAELAAEWLERGGREEAYIAVVIAQLVEAAE |
| | | | RRGSEELLKIAELAAEWLERGGREEAYIAVVIAQLVEAAERRGSEELLKIAELAAEWL |
| | | | ERGGREEAYIAVVIAQLVEAAERRGSEELLRIAEEAAEWTERGGREEAYHAVVRAQLV |
| | | | EAAERKGT |
| 76 | IP_b1_154 | R6_20 | PEEEAHKKTWKRITAAFDFGKREEELEKILEEAIRELEKNGVDREEAAHRATWILITA |
| | | | AFDFGKDDRELEKILEIAIRILEKNGVDREEAAHRATWILITAAFDFGKDDRELEKIL |
| | | | EIAIRILEKNGVDREEAAHRATWILITAAFDFGKDDRELEKILEIAIRILEKNGVDRE |
| | | | EAAHRATWILITAAFDFGKDDRELEKILEIAIRILEKNGVDRREAAKRATWILRTAAH |
| | | | DFGKHEEEEERIEEIAKRILEKNGHE |
| 77 | IP_b1_155 | R6_CP33 | PEEKLYWEVSQIAFSAIEEQKNGGDEHVEKALKKVKKAAEELGNSEEEALYWLVSQIA |
| | | | FSAIELQKNGGDEYVEAALKVVKKAAEELGNSEEEALYWLVSQIAFSAIELQKNGGDE |
| | | | YVEAALKVVKKAAEELGNSEEEALYWLVSQIAFSAIELQKNGGDEYVEAALKVVKKAA |
| | | | EELGNSEEEALYWLVSQIAFSAIELQKNGGDEYVEAALKVVKKAAEELGNSEDEALHL |
| | | | LKSQIAFSAHELENNGGEEYVKAAEKVEKKAAEELGNE |
| 78 | IP_b1_156 | R6_PO11 | PEEERIKYVITVVEQIAKDAHRNGQEELAKLAERTAEEAKKATERGEEETLRIVYVIV |
| | | | VVLQIALEAHRNGQEELAKLALRTAEEAIKATERGEEETLRIVYVIVVVLQIALEAHR |
| | | | NGQEELAKLALRTAEEAIKATERGEEETLRIVYVIVVVLQIALEAHRNGQEELAKLAL |
| | | | RTAEEAIKATERGEEETLRIVYVIVVVLQIALEAHRNGQEELAKLALRTAEEAIKATE |
| | | | RGEEETERIVYDIVVVLQEALEAHRNGEEERAKKALDEARRRIEATERGE |
| 79 | IP_b1_157 | R6_n11 | PEREKVEKLARNAHLWAKKKLEEAKRHGNEEETEEWKRETELVRRLAELAHKSDREEV |
| | | | LKVIKLALNALLWANKKLDEAKRHGNEEETEEWRKETELVKRLAELASKSDREEVLKV |
| | | | IKLALNALLWANKKLDEAKRHGNEEETEEWRKETELVKRLAELASKSDREEVLKVIKL |
| | | | ALNALLWANKKLDEAKRHGNEEETEEWRKETELVKRLAELASKSDREEVLKVIKLALN |
| | | | ALLWANKKLDEAKRHGNEEETEEWRKETELVKRLAELASKSDREEVLKTIKLALNNLL |
| | | | WANKKLDEAKRHGNEEETEHWRKRTETVKREAEKASKSDK |
| 80 | IP_b1_158 | R6_n6 | PEEEAHKKANVRISVAHEYGKREEELEKILEEAIRELEKNGVDREEAAHRAANVLISV |
| | | | AHEYGKDDRELEKILEIAIRILEKNGVDREEAAHRAANVLISVAHEYGKDDRELEKIL |
| | | | EIAIRILEKNGVDREEAAHRAANVLISVAHEYGKDDRELEKILEIAIRILEKNGVDRE |
| | | | EAAHRAANVLISVAHEYGKDDRELEKILEIAIRILEKNGVDRREAAKRAANVLISVAH |
| | | | EYGKHEEEEERIEEIAKRILEKNGHE |
| 81 | IP_b1_159 | R6_new_ | PEEEAHKKAWKRITTAHQYGKREEELEKILEEAIRELEKNGVDREEAAHRAAWILITT |
| | | 01 | AHQYGKDDRELEKILEIAIRILEKNGVDREEAAHRAAWILITTAHQYGKDDRELEKIL |
| | | | EIAIRILEKNGVDREEAAHRAAWILITTAHQYGKDDRELEKILEIAIRILEKNGVDRE |
| | | | EAAHRAAWILITTAHQYGKDDRELEKILEIAIRILEKNGVDRREAAKRAAWILRTTAH |
| | | | QYGKHEEEEERIEEIAKRILEKNGHE |
| 82 | IP_b1_160 | R6_new_ | PEEEAHKKAWKRIVIAHQYGKREEELEKILEEAIRELEKNGVDREEAAHRAAWILIVI |
| | | 02 | AHQYGKDDRELEKILEIAIRILEKNGVDREEAAHRAAWILIVIAHQYGKDDRELEKIL |
| | | | EIAIRILEKNGVDREEAAHRAAWILIVIAHQYGKDDRELEKILEIAIRILEKNGVDRE |
| | | | EAAHRAAWILIVIAHQYGKDDRELEKILEIAIRILEKNGVDRREAAKRAAWILRVIAH |
| | | | QYGKHEEEEERIEEIAKRILEKNGHE |
| 83 | IP_b1_161 | R6_new_ | PEEEATKKASERIVQAHMYGKREEELEKILEEAIRELEKNGVDREEAATRAASELIVQ |
| | | 03 | AHMYGKDDRELEKILEIAIRILEKNGVDREEAATRAASELIVQAHMYGKDDRELEKIL |
| | | | EIAIRILEKNGVDREEAATRAASELIVQAHMYGKDDRELEKILEIAIRILEKNGVDRE |
| | | | EAATRAASELIVQAHMYGKDDRELEKILEIAIRILEKNGVDRREAAKRAASELRVQAH |
| | | | MYGKHEEEEERIEEIAKRILEKNGHE |
| 84 | IP_b1_162 | R6_new_ | PEEEAHKKAFLRIDVADDYGKREEELEKILEEVIRELEKNGVDREEAAHRAAFLLIDV |
| | | 04 | ADDYGKDDRELEKILEIVIRILEKNGVDREEAAHRAAFLLIDVADDYGKDDRELEKIL |
| | | | EIVIRILEKNGVDREEAAHRAAFLLIDVADDYGKDDRELEKILEIVIRILEKNGVDRE |
| | | | EAAHRAAFLLIDVADDYGKDDRELEKILEIVIRILEKNGVDRREAAKPAAFLLRDVAD |
| | | | DYGKHEEEEERIEEIVKRILEKNGHE |
| 85 | IP_b1_163 | R6_new_ | PEEEAHKKAMIRIDVADDYGKREEELEKILEEAIRELEKNGVDREEAAHRAAMILIDV |
| | | 05 | ADDYGKDDRELEKILEIAIRILEKNGVDREEAAHRAAMILIDVADDYGKDDRELEKIL |
| | | | EIAIRILEKNGVDREEAAHRAAMILIDVADDYGKDDRELEKILEIAIRILEKNGVDRE |
| | | | EAAHRAAMILIDVADDYGKDDRELEKILEIAIRILEKNGVDRREAAKRAAMILRDVAD |
| | | | DYGKHEEEEERIEEIAKRILEKNGHE |
| 86 | IP_b1_166 | R6_new_ | PEEEKRTKHLEEVAETVRERAKEVIDKDNDEEVKNLAVWAEEAAHTVEEALKTARNDT |
| | | 07 | GKEEKERLDHLVEVASTVIERAIEVIEKDNDKEVINLAVWAIEAAVTVLEALRTARND |
| | | | TGKEEKERLDHLVEVASTVIERAIEVIEKDNDKEVINLAVWAIEAAVTVLEALRTARN |
| | | | DTGKEEKERLDHLVEVASTVIERAIEVIEKDNDKEVINLAVWAIEAAVTVLEALRTAR |
| | | | NDTGKEEKERLDHLVEVASTVIERAIEVIEKDNDKEVINLAVWAIEAAVTVLEALRTA |
| | | | PNDTGKEEKERLDHLVEEASRVIEEAIKEIEKDNDKRVLNLAVWKIEKAVEELKRERT |
| | | | ERNDKN |
| 87 | IP_b1_167 | R6_new_ | PEEEAYLKAQKRIDTAHEHGKREEELEKILEEVIRELEKNGVDREEAAYLAAQILIDT |
| | | 08 | AHEQGKDDRELEKILEIVIRILEKNGVDREEAAYLAAQILIDTAHEQGKDDRELEKIL |
| | | | EIVIRILEKNGVDREEAAYLAAQILIDTAHEQGKDDRELEKILEIVIRILEKNGVDRE |
| | | | EAAYLAAQILIDTAHEQGKDDRELEKILEIVIRILEKNGVDRREAAYLAAQILRDTAR |
| | | | EQGKHEEEEERIEEIVKRILEKNGHE |
| 88 | IP_b1_168 | R6_new_ | PDEEAEKHIEEVVRRARENGSSEYEALFEASQEVASKIFEKIKRNGGDDREAAETAIE |
| | | 09 | VVVRAARKGGSSEYEALFEASQAVASKIFEHIKRNGGDDREAAETAIEVVVRAARKGG |
| | | | SSEYEALFEASQAVASKIFEHIKRNGGDDREAAETAIEVVVRAARKGGSSEYEALFEA |
| | | | SQAVASKIFEHIKRNGGDDREAAETAIEVVVRAARKGGSSEYEALFEASQAVASKIFE |
| | | | HIKRNGGDDREAAKTAKEVVVRAARKGGSSEYEAEFEASQAVKSKEEEHRRRNGDS |
| 89 | IP_b1_169 | R6_new_ | PEHVIKRAVMEAVNRAETEKENGGDEEEALRLAAEVVIHVVEEEGGSEEWVIKAAVMV |
| | | 10 | AVNLAETEKENGGDKEEALRLAAEVVINVVENSGGSEEWVIKAAVMVAVNLAETEKEN |
| | | | GGDKEEALRLAAEVVINVVENSGGSEEWVIKAAVMVAVNLAETEKENGGDKEEALRLA |
| | | | AEVVINVVENSGGSEEWVIKAAVMVAVNLAETEKENGGDKEEALRLAAEVVINVVENS |
| | | | GGSEEWVEKAAVMVAVNLAETEKENGGDEEEAERLAEEVRRNVVERSGGS |
| 90 | IP_b1_170 | R6_new_ | PEREKVEKLARNAHLWAKKKLEEAKRHGNEEETEEWKRETELVRRLAELAHKSDREEV |
| | | 11 | LKVIKLALNALLWANKKLDEAKRHGNEEETEEWRKETELVKRLAELASKSDREEVLKV |
| | | | IKLALNALLWANKKLDEAKRHGNEEETEEWRKETELVKRLAELASKSDREEVLKVIKL |
| | | | ALNALLWANKKLDEAKRHGNEEETEEWRKETELVKRLAELASKSDREEVLKVIKLALN |
| | | | ALLWANKKLDEAKRHGNEEETEEWRKETELVKRLAELASKSDREEVLKTIKLALNNLL |
| | | | WANKKLDEAKRHGNEEETEHWRKRTETVKREAEKASKSDK |
| 91 | IP_b1_172 | s23-E2 | DEEREKLKEKLKEVLRRAKEAKKKGDKEKLIELAYEAAALAAWIIHKDSNDDEIVELA |
| | | | KEALKLVLEAAKEAKKNGDKEKLIKLAYLAAAVAKWIIHTDGDDDEIVELAKEALKLV |
| | | | LEAAKEAKKNGDKEKLIKLAKLAASVAKWIIHTDGDDDEIVELAKEALKLVLEAAKEA |
| | | | KKNGDKEKLIKLAKLAASVAAWIIHTDGDDDEIVELAKEALKLVLEAAKEAKKNGDKE |
| | | | KLIKLAYLAAAVAAWIITTDGDDEEIVELAKEALKLVKEAAEEAEKQGDEELREKLRY |
| | | | LSEAVREWIERNDGDD |
| 92 | IP_b1_174 | s25 | PEEEIVEEAVNEASEITEKTKDEEKIKEALERILREAAERIRENGGNEEEAILEIAVN |
| | | | VASRITERVKNEEEIKKALEWILRVAAELIRENGGNEEEAILVIAINVASRITERVKN |
| | | | EEEIKKALEWILRVAAELIRENGGNEEEAIVVIAINVASRITERVKNEEEIKKALEWI |
| | | | LRVAAELIRENGGNEEEAIVEIAINVASRITERVKNEEEIKKALEWILRVAAELIREN |
| | | | GGNEEEAILEIAVNVASRITKRVTNEEEIKKAQHWIKHVAKKLIKENGGT |
| 93 | IP_b1_175 | s59 | PEEEILVKAVNEASRITEKTKDEEKIKEALERILREAAERIRENGGNEEEAILVIAVN |
| | | | VASRITERVKNEEEIKKALEWILRVAAELIRENGGNEEEAISVIAINVASRITERVKN |
| | | | EEEIKKALEWILRVAAELIRENGGNEEEAIVEIAINVASRITERVKNEEEIKKALEWI |
| | | | LRVAAELIRENGGNEEEAIVEIAINVASRITERVKNEEEIKKALEWILRVAAELIREN |
| | | | GGNEEEAILEIAVNVASRITKRVTNEEEIKKAQHWIKHVAKKLIKENGGT |
| 94 | IP_b1_178 | s93 | PEEEILIKAVNEASRITEKTKDEEKIKEALERILREAAERIRENGGNEEEAILIIAVN |
| | | | VASRITERVKNEEEIKKALEWILRVAAELIRENGGNEEDAISIIAINVASRITERVKN |
| | | | EEEIKKALEWILRVAAELIRENGGNEEDAIVEIAINVASRITERVKNEEEIKKALEWI |
| | | | LRVAAELIRENGGNEEDAIVEIAINVASRITERVKNEEEIKKALEWILRVAAELIREN |
| | | | GGNEEDAILEIAVNVASRITKRVTNEEEIKKAQHWIKHVAKKLIKENGGT |
| 95 | IP_b1_181 | s1_S2_06 | PEEEAYTKASIKIQHAFDEGKKEEELEKILEEAIKELEKNGVDKEEAAYIAASILIQS |
| | | | AFDTGKDDKELEKILEIAIKILEKNGVDKEEAAYIAASILIQSAFDTGKDDKELEKIL |
| | | | EIAIKILEKNGVDKKEAAYIAASILIQSAKDTGKHEEEEEKIEEIAKKILEKNGHE |
| 96 | IP_b1_182 | s1_S2_24 | PEEEIKKKAVNEASRITEKTKDEEKIKEALERILREAAERIRENGGNEEEAILVIAVN |
| | | | VASRITERVKNEEEIKKALEWILRVAAELIRENGGNEEEAILVIAINVASRITERVKN |
| | | | EEEIKKALEWILRVAAELIRENGGNEEEAIVTIAINVASRITERVKNEEEIKKALEWI |
| | | | LRVAAELIRENGGNEEEAIVEIAINVASRITERVKNEEEIKKALEWILRVAAELIREN |
| | | | GGNEEEAILEIAVNVASRITKRVTNEEEIKKAQHWIKHVAKKLIKENGGT |
| 97 | IP_b1_184 | s1_S2_42 | PEEEIIIKAANEAFEIVEKTKDEEKIKEALERILREAAERIRENGGNEEEAIVLIAVN |
| | | | VASYIVEDVKNEEEIKKALEWILRVAAELIRENGGNEEDAIVVIAVNVASRITEDVKN |
| | | | EEEIKKALEWILRVAAELIRENGGNEEDAIVVIAINVASRITERVKNEEEIKKALEWI |
| | | | LRVAAELIRENGGNEEDAIVEIAINVASRITERVKNEEEIKKALEWILRVAAELIREN |
| | | | GGNEEDAILEIAVNVASRITKRVTNEEEIKKAQHWIKHVAKKLIKENGGT |

TABLE 3

| SEQ | | |
|---|---|---|
| ID NO | Name | Sequence |
| 98 | endo1_b1_ | DEIVELAKEALKLVLEAAKEAKKNGDKEKLIKLAYLAAAVAAWIIHTDGDDDEIVELAKEALKLVLE |
| | 01 | AAKEAKKNGDKEKLIKLAYLAAAVAAWIIHTDGDDDEIVELAKEALKLVLEAAKEAKKNGDKEKLIK |
| | | LAYLAAAVAAWIIHTDGDDDEIVELAKEALKLVLEAAKEAKKNGDKEKLIKLAYLAAAVAAWIIHTD |
| | | GDDDEIVELAKEALKLVLEAAKEAKKNGDKEKLIKLAYLAAAVAAWIITTDGDDDEIVELAKEALKL |
| | | VLEAAKEAKKNGDKEKLIKLAYLAAAVAAWIIHTDGDDDEIVELAKEALKLVLEAAKEAKKNGDKEK |
| | | LIKLAYLAASVAAWIITTDGDDEEIVELAKEALKLVKEAAEEAEKQGDEELREKLRYLSEAVREWIE |
| | | RNDGDD |
| 99 | endo1_b1_ | DEIVELAKEALKLVLEAAKEAKKNGDKEKLIKLAYLAAAVAAWIIHTDGDDDEIVELAKEALKLVLE |
| | 02 | AAKEAKKNGDKEKLIKLAYLAAAVAAWIIHTDGDDDEIVELAKEALKLVLEAAKEAKKNGDKEKLIK |
| | | LAYLAAAVAAWIIHTDGDDDEIVELAKEALKLVLEAAKEAKKNGDKEKLIKLAYLAAAVAAWIIHTD |
| | | GDDDEIVELAKEALKLVLEAAKEAKKNGDKEKLIKLAYLAAAVAAWIIKTDGDDDEIVELAKEALKL |
| | | VLEAAKEAKKNGDKEKLIKLAYLAAAVAAWIIHTDGDDDEIVELAKEALKLVLEAAKEAKKNGDKEK |
| | | LIKLAYLAASVAAWIITTDGDDEEIVELAKEALKLVKEAAEEAEKQGDEELREKLRYLSEAVREWIE |
| | | RNDGDD |
| 100 | endo1_b1_ | DEIVELAKEALKLVLEAAKEAKKNGDKEKLIKLAYLAAAVAAWIIHTDGDDDEIVELAKEALKLVLE |
| | 03 | AAKEAKKNGDKEKLIKLAYLAAAVAAWIIHTDGDDDEIVELAKEALKLVLEAAKEAKKNGDKEKLIK |
| | | LAYLAAAVAAWIIHTDGDDDEIVELAKEALKLVLEAAKEAKKNGDKEKLIKLAYLAAAVAAWIIHTD |
| | | GDDDEIVELAKEALKLVLEAAKEAKKNGDKEKLIKLAYLAAAVAAWIIITDGDDDEIVELAKEALKL |
| | | VLEAAKEAKKNGDKEKLIKLAYLAAAVAAWIIHTDGDDDEIVELAKEALKLVLEAAKEAKKNGDKEK |
| | | LIKLAYLAASVAAWIITTDGDDEEIVELAKEALKLVKEAAEEAEKQGDEELREKLRYLSEAVREWIE |
| | | RNDGDD |
| 101 | endo1_b1_ | EIVELAKEALKLVLEAAKEAKKNGDKEKLIKLAYLAAAVAAWIIHTDGDDDEIVELAKEALKLVLEA |
| | 04 | AKEAKKNGDKEKLIKLAYLAAAVAAWIIHTDGDDDEIVELAKEALKLVLEAAKEAKKNGDKEKLIKL |
| | | AYLAAAVAAWIIHTDGDDDEIVELAKEALKLVLEAAKEAKKNGDKEKLIKLAYLAAAVAAWIIITDG |
| | | DDDEIVELAKEALKLVLEAAKEAKKNGDKEKLIKLAYLAAAVAAWIIHTDGDDDEIVELAKEALKLV |
| | | LEAAKEAKKNGDKEKLIKLAYLAASVAAWIITTDGDDEEIVELAKEALKLVKEAAEEAEKQGDEELR |
| | | EKLRYLSEAVREWIERNDGDD |
| 102 | endo1_b1_ | EKVELAKEKLKEVLEEAKEAKKNGDKEKLIKKAYTAAAVAAWIIHTDGDDDEIVELAKEALKLVLEA |
| | 05 | AKEAKKNGDKEKLIKLAYLAAAVAAWIIHTDGDDDEIVELAKEALKLVLEAAKEAKKNGDKEKLIKL |
| | | AYLAAAVAAWIIHTDGDDDEIVELAKEALKLVLEAAKEAKKNGDKEKLIKLAYLAAAVAAWIIITDG |
| | | DDDEIVELAKEALKLVLEAAKEAKKNGDKEKLIKLAYLAAAVAAWIIHTDGDDDEIVELAKEALKLV |
| | | LEAAKEAKKNGDKEKLIKLAYLAASVAAWIITIDGDDEEIVELAKEALKLVKEAAEEAEKQGDEELR |
| | | EKLRYLSEAVREWIERNDGDD |
| 103 | endo1_b1_ | KEKKIKKAYTAAAEAAWKIHTDGDDDEIVEEAKEALKKVLEAAKEAKKNGDKEKLIKLAYLAAAVAA |
| | 06 | WIIHTDGDDDEIVELAKEALKLVLEAAKEAKKNGDKEKLIKLAYLAAAVAAWIIHTDGDDDEIVELA |
| | | KEALKLVLEAAKEAKKNGDKEKLIKLAYLAAAVAAWIIITDGDDDEIVELAKEALKLVLEAAKEAKK |
| | | NGDKEKLIKLAYLAAAVAAWIIHTDGDDDEIVELAKEALKLVLEAAKEAKKNGDKEKLIKLAYLAAS |
| | | VAAWIITTDGDDEEIVELAKEALKLVKEAAEEAEKQGDEELREKLRYLSEAVREWIERNDGDD |
| 104 | endo1_b1_ | KEKKIEKAYRAAAEAAWKIHTDGDDDEIVEEAKEALKKVLEAAKEAKKNGDKEKLIKLAYLAAAVAA |
| | 07 | WIIHTDGDDDEIVELAKEALKLVLEAAKEAKKNGDKEKLIKLAYLAAAVAAWIIHTDGDDDEIVELA |
| | | KEALKLVLEAAKEAKKNGDKEKLIKLAYLAAAVAAWIIITDGDDDEIVELAKEALKLVLEAAKEAKK |
| | | NGDKEKLIKLAYLAAAVAAWIIHTDGDDDEIVELAKEALKLVLEAAKEAKKNGDKEKLIKLAYLAAS |
| | | VAAWIITTDGDDEEIVELAKEALKLVKEAAEEAEKQGDEELREKLRYLSEAVREWIERNDGDD |
| 105 | endo1_b1_ | KEKKIEKAYRAAAEAAWKIHTDGDDDEIVEEAKEALKKVLEAAKEAKKNGDKEKLIKLAYLAAAVAA |
| | 08 | WIIHTDGDDDEIVELAKEALKLVLEAAKEAKKNGDKEKLIKLAYLAAAVAAWIIHTDGDDDEIVELA |
| | | KEALKLVLEAAKEAKKNGDKEKLIKLAYLAAAVAAWIIITDGDDDEIVELAKEALKLVLEAAKEAKK |
| | | NGDKEKLIKLAYLAAAVAAWIIHTDGDDDEIVELAKEALKLVLEAAKEAKKNGDKEKLIYLAYLAAS |
| | | VAAWIITTDGDDEEIVELAKEALKLVKEAAEEAEKQGDEELREKLRYLSEAVREWIERNDGDD |

In one embodiment, any N-terminal methionine residue is deleted and is not considered in determining percent identity relative to the reference protein. In another embodiment, any N-terminal methionine residue is present and is considered in determining percent identity relative to the reference protein. In other embodiments, 1, 2, 3, 4, or 5 amino acids at the N-terminus and/or C-terminus of the polypeptide may be absent, and not considered in determining percent identity relative to the reference protein.

In one embodiment substitutions relative to the reference sequence are conservative amino acid substitutions. As used herein, a “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Particular conservative substitutions include, but are not limited to, Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gin or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

The polypeptides of the disclosure may comprise further amino acids, such as additional amino acids at the N-terminus and/or C-terminus, which are not considered when determining percent identity relative to the reference polypeptide. In one embodiment, the polypeptides further comprises one or more functional domains. The polypeptides may comprise any further functional domain fused to the polypeptide that may be of use for an intended purpose. In various non-limiting embodiments, the resulting fusion protein comprises an additional functional domain such as detectable proteins, purification tags, protein antigens, and protein therapeutics. The functional domain may be a genetic fusion or may be otherwise covalently linked to the polypeptide.

As used throughout the present application, the term “polypeptide” is used in its broadest sense to refer to a sequence of subunit amino acids. The polypeptides of the invention may comprise L-amino acids+glycine, D-amino acids+glycine (which are resistant to L-amino acid-specific proteases in vivo), or a combination of D- and L-amino acids+glycine. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

In another embodiment, the disclosure comprises a library comprising a plurality (at least 2, 5, 10, 25, 50, 75, 100, or more) of different polypeptides of any embodiment or combination of embodiments herein.

The disclosure also provides nucleic acids encoding the polypeptide of any embodiment or combination of embodiments of the disclosure. The nucleic acid sequence may comprise single stranded or double stranded RNA (such as an mRNA) or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to poly A sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptides, oligomers, nucleic acids, expression vectors (i.e.: episomal or chromosomally integrated), disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-polycationic mediated-, or viral mediated transfection.

In one embodiment, the disclosure provides pharmaceutical compositions comprising (a) the polypeptide, nucleic acid, expression vector, and/or host cell of any preceding claim; and (b) a pharmaceutically acceptable carrier. In one embodiment, the polypeptide, nucleic acid, expression vector, and/or host cell comprise or encode a polypeptide comprising one or more functional domain selected from protein antigens and protein therapeutics. The composition may also comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The disclosure further provides methods for use of the polypeptide, nucleic acid, expression vector, library, and/or host cell of any embodiment herein for any suitable purpose. In one embodiment, the methods comprise use as synthetic peptide ligands for receptor activation. In another embodiment, the polypeptide comprises an additional functional domain comprising a protein antigen and/or protein therapeutics, and the methods may comprise a method for treating a disorder that the protein therapeutic is effective against, or generating an immune response against the protein antigen.

The disclosure also provides methods for deigning a peptide binding polypeptide, comprising any combination of steps as disclosed in the examples.

### Examples

General approaches for designing sequence-specific peptide binding proteins would have wide utility in proteomics and synthetic biology. Although considerable progress has been made in designing proteins which bind to other proteins, the general peptide binding problem is more challenging as most peptides do not have defined structures in isolation, and to offset the loss in solvation upon binding the protein binding interface has to provide specific hydrogen bonds that complement the majority of the buried peptide's backbone polar groups. We describe a general approach for de novo design of proteins made out of repeating units that bind peptides with repeating sequences such that there is a one to one correspondence between repeat units on the protein and peptide. We develop a rapid docking plus geometric hashing method to identify protein backbones and protein-peptide rigid body arrangements that are compatible with bidentate hydrogen bonds between side chains on the protein and the backbone of the peptide; the remainder of the protein sequence is then designed using Rosetta™ to incorporate additional interactions with the peptide and drive folding to the desired structure. We use this approach to design, from scratch, alpha helical repeat proteins that bind six different tripeptide repeat sequences (PLP, LRP, PEW, IYP, PRM and PKW) in near polyproline 2 helical conformations. The proteins are expressed at high levels in E. coli, are hyperstable, and bind peptides with 4-6 copies of the target tripeptide sequences with nanomolar to picomolar affinities both in vitro and in living cells. Crystal structures reveal repeating interactions between protein and peptide interactions as designed, including a ladder of protein sidechain to peptide backbone hydrogen bonds. By redesigning the binding interfaces of individual repeat units, specificity can be achieved for non-repeating sequences, and for naturally occurring proteins containing disordered regions. Our approach provides a general route to designing specific binding proteins for a broad range of repeating and non-repetitive peptide sequences.

We set out to generalize peptide recognition by modular repeat-protein scaffolds to arbitrary repeating peptide backbone geometries. This requires solving two main challenges: building protein structures with a repeat spacing and orientation matching that of the target peptide conformation, and ensuring the replacement of peptide-water hydrogen bonds in the unbound state with peptide-protein hydrogen bonds in the bound state. The first challenge is critical for modular and extensible sequence recognition: if individual repeat units in the protein are to bind individual repeat units on the peptide in the same orientation, the geometric phasing of the repeat units on protein and peptide must be compatible. The second challenge is critical for achieving high binding affinity: in conformations other than the alpha and 3-10 helix, the NH and C═O groups make hydrogen bonds with water in the unbound state that need to be replaced with hydrogen bonds to the protein upon binding to avoid incurring a substantial free energy penalty.

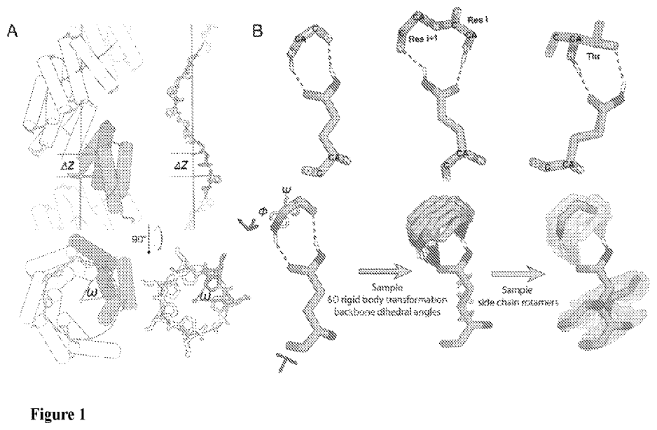

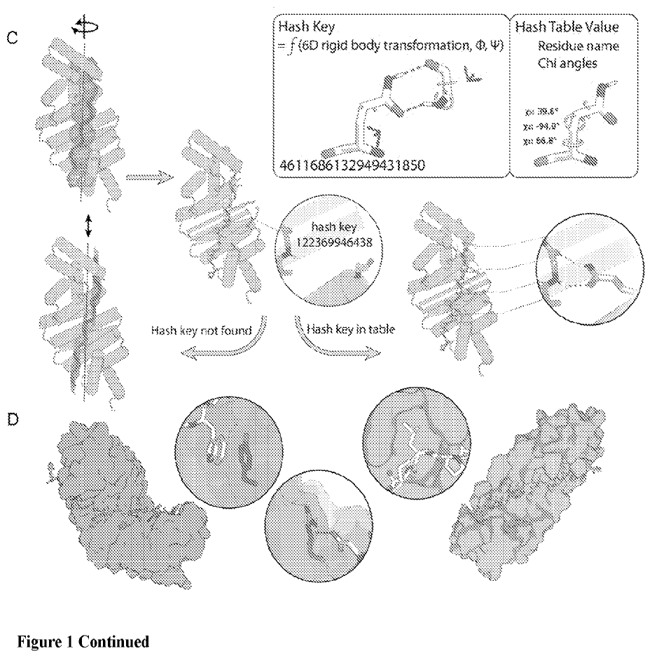

To address the first challenge, we reasoned that a necessary (but not sufficient) criterion for in-phase geometric matching between repeating units on designed protein and peptide was a correspondence between the superhelices that the two trace out. All repeating polymeric structures trace out superhelices which can be described by three parameters: the translation (rise) along the helical axis per repeat unit, the rotation (twist) around this axis, and the distance (radius) of the repeat unit centroid from the axis (FIG. 1A). As described in the methods, we generated large sets of repeating protein backbones sampling a wide range of superhelical geometries. We generated corresponding sets of repeating peptide backbones by randomly sampling di-peptide and tri-peptide conformations in allowed regions of the Ramachandran map (avoiding intra-peptide steric clashes), and then repeating these four to six times to generate 12-24 residue peptides. We then searched for matching pairs of repeat protein and repeat peptide backbones, requiring that the rise be within 0.2A, the twist within 5 degrees, and the radius differ by at least 4A (the difference in radius is necessary to avoid clashing between peptide and protein; the peptide can wrap either outside or inside the protein).

To address the second challenge, we reasoned that bidentate hydrogen bonds between side chains on the protein and pairs of backbone groups or backbone and sidechain groups on the peptide could allow the burying of sufficient peptide surface area on the protein to achieve high affinity binding without incurring a large desolvation penalty. As the geometric requirements for such bidentate hydrogen bonds are quite strict, we developed a geometric hashing approach to enable rapid identification of rigid body docks of the peptide on the protein compatible with ladders of bidentate interactions. To generate the hash tables for bidentate sidechain-backbone interactions, Monte Carlo simulations of individual sidechain functional groups making bidentate hydrogen bonding interactions with peptide backbone and/or sidechain groups were carried out using the Rosetta™ energy function (12), and a move set consisting of both rigid body perturbations and changes to the peptide backbone torsions (FIG. 1B; see Methods for details). For each accepted (low energy) arrangement, sidechain rotamer conformations were built backwards from the functional group to identify the possible placements of the protein backbone from which the bidentate interaction could be realized. The results of these calculations were stored in hash tables: for each placement, a hash key was computed from the rigid body transformation and peptide backbone and side chain torsion angles determining the position of the hydrogen bonding groups (for example the phi and psi torsion angles for a bidentate hydrogen bond to the NH and CO groups of the same amino acid), and the chi angles of the corresponding rotamer were stored in the hash for this key (18). Hash tables were generated for ASN and GLN making bidentate interactions with the N—H and C═O groups on the backbone of a single residue or adjacent residues, ASP or GLU making bidentate interactions with the N—H groups of two successive amino acids, and for sidechain-sidechain pi-pi and cation-pi interactions (see Methods).

To identify rigid body docks that enable multiple bidentate hydrogen bonds between repeat protein and peptide, we took advantage of the fact that for matching two superhelical structures along their common axis, there are only two degrees of freedom: the translational and rotational offsets of one super helix to the other. For each repeat protein-repeat peptide pair, we carried out a grid search in these two degrees of freedom, sampling relative translations and rotations in ˜1 Å and 10 degree increments (FIG. 1E). For each generated dock, we computed the rigid body orientation for each peptide-protein residue pair, and queried the hash tables to very rapidly determine if these were suitable for any of the bidentate interactions; docks for which there were lower than a threshold number of matches were discarded. For the remaining docks, following building of the interacting side chains using the chi angle information stored in the hash, and rigid body minimization to optimize hydrogen bond geometry, we used Rosetta™ combinatorial optimization to design the protein and peptide sequences (20), keeping the residues identified in the hash matching fixed, and enforcing sequence identity between repeats in both peptide and protein (see Methods).

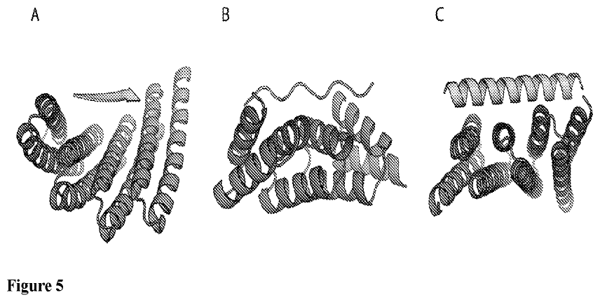

In initial calculations with unrestricted sampling of peptide conformations, designs were generated with a wide range of peptide conformations. Examples of repeat proteins designed to bind to extended beta strand, polypeptide II, and helical peptide backbones, as well as a range of less canonical structures are shown in FIG. 5. Reasoning that proline containing peptides would incur a lower entropic cost upon binding, we decided to start experimental characterization with designs containing at least one proline residue; in most such designs the peptide backbone is in or near the polyproline II portion of the Ramachandran map. Our design strategy requires matching the twist of the repeat unit of the peptide with that of the protein, and hence choosing a repeat length of the peptide that generates close to a full 360 degree turn requires less of a twist in the repeat protein; for the polyproline helix there are roughly 3 residues per turn and likely because of this we obtained more designs which target 3 residue than 2 residue proline containing repeat units. We selected for experimental characterization 43 designed complexes with near ideal bidentate hydrogen bonds between protein and peptide, favorable protein-peptide interaction energies (12), interface shape complementary (21), and few interface unsatisfied hydrogen bonds (22) which consistently retained more than 80% of the interchain hydrogen bonds in 20 ns molecular dynamics trajectories.

We obtained synthetic genes encoding the designed proteins with a terminal biotinylation tag, expressed the proteins in E. coli, and purified them by Ni-NTA chromatography. 30 of 49 were monomeric and soluble. To assess binding, the target peptides were displayed on the yeast cell surface (23), and binding to the repeat proteins was monitored by flow cytometry. To obtain a complete readout of the peptide binding specificity of individual designs, we in parallel used large scale array based oligonucleotide synthesis to generate yeast display libraries encoding all 2 and 3 residue repeat peptides with 8 repeat units each, and used fluorescence activated cell sorting (FACS) followed by Sanger sequencing to identify the peptides recognized by each designed protein. Many of the designs bound peptides with sequences similar to those targeted but the affinity and specificity were both relatively low, with most of the successes for 3 residue repeat units.

Based on these results, we sought to increase the peptide sequence specificity of the computational design protocol, focusing on design of binders for peptides with 3 residue repeat units. First, we required that each non-proline residue in the peptide make specific contacts with the protein, and that the pockets and grooves engaging sidechains emanating on the two sides of the peptide were quite distinct. Second, following design, we evaluated the change in binding energy (Rosetta™ DDG) (24) for all single residue changes to the peptide repeating unit, and selected only designs for which the design target sequence made the most favorable interactions with the designed protein. Third, we used computational Alanine scanning to remove hydrophobic residues on the protein surface not contributing to binding specificity to decrease non-specific binding (25). Fourth, to assess the structural specificity of the designed peptide binding interface, we carried out Monte Carlo flexible backbone docking calculations, starting from large numbers of peptide conformations with superhelical parameters in the range of those of the proteins, and selected those designs with converged peptide backbones (RMSD<2.0 among the top 20 designs with lowest DDG) close to the design model (RMSD<1.5).

We tested 54 second-round designed protein-peptide pairs using the yeast flow cytometry assay described above. 42 of the designed proteins were solubly expressed in E. coli, and 16 designed bound their targets with considerably higher affinity and specificity than in the first round. We selected six designs with diverse superhelical parameters and shapes, and a range of target peptides for more detailed characterization (PLPx6; LRPx6; PEWx6; TYPx6; PRMx6; and PKWx6). As evident in the design models, these are six repeat proteins with a one to one match between repeat units in the protein and in the target peptide. Small Angle X-ray Scattering (SAXS) profiles (26, 27) were close to those computed from the design models, suggesting that the proteins fold into the designed shapes in solution. Circular dichroism (CD) studies showed that all six are largely helical and thermostable up to 95° C. Bio-Layer Interferometry (BLI) characterization of binding to biotinylated target peptides immobilized on Octet™ sensor chips revealed dissociation constant (Kd) values ranging from <500 μM to ˜40 nM; five out of six with dissociation half-life>=500s. Little decrease in binding was observed after storage of the proteins for 30 days at 4° C. Three out of the six designs showed little dissociation after 1,000-2,000s in buffer, indicating the Kd is too tight to be accurately measured with BLI. The binding surfaces of several related designs were subjected to Site Saturation Mutagenesis (SSM) (28) on yeast; and following incorporation of 1-3 enriched substitutions strong binding signals were obtained in flow cytometry using only 10 μM biotinylated cognate peptides for 2 designs (RPB_PL1-R6 and RPB_PEW1_R6).

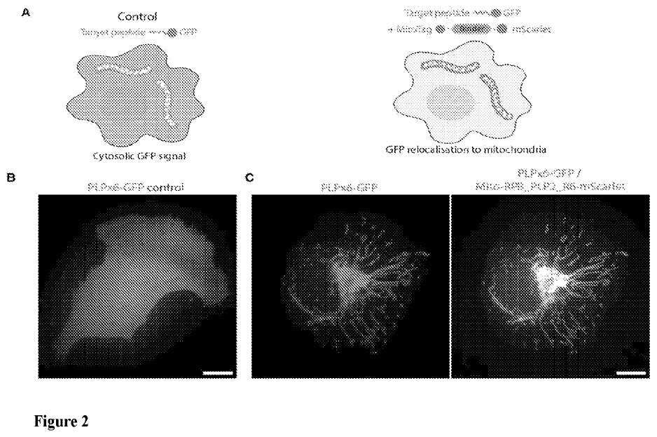

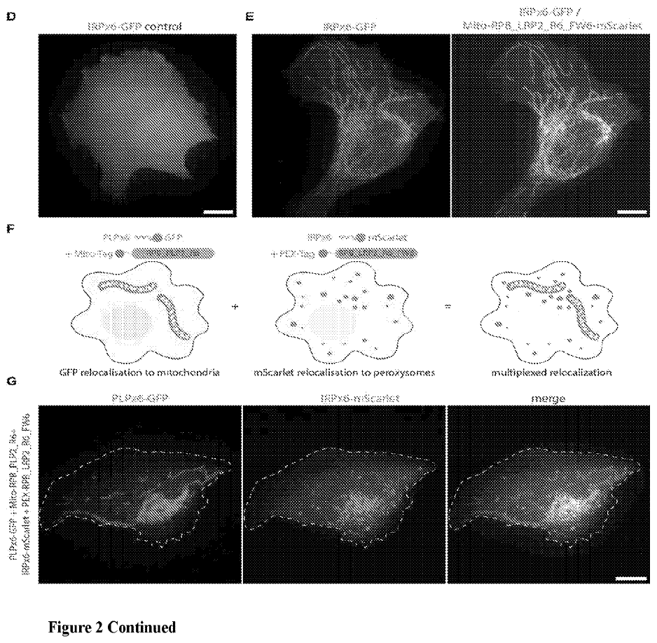

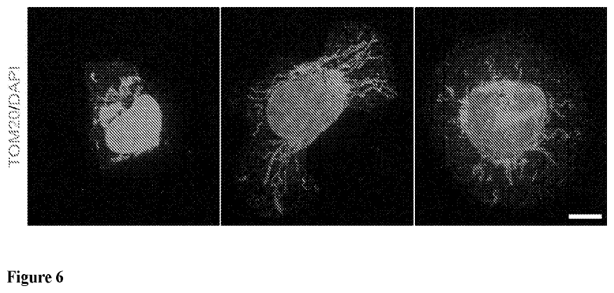

Many current cell biology approaches (29) involve tagging cellular target proteins with a protein or peptide, and then introducing into the same cell a protein which binds the tag with high affinity and specificity, but does not bind endogenous targets. A bottleneck in such studies is that binders obtained from antibody-scaffold (scFV or VHH) based library screens often do not fold properly in the reducing environment of the cytosol, resulting in loss of binding (30). We reasoned that our binders would not have this limitation as they are designed for stability and lack disulfide bonds. As a proof of concept, we coexpressed the peptide PLPx6 fused to GFP and its cognate binder, RPB_PLP2_R6, a variant of RPB_PLP1_R6, fused to both mScarlet™ and a targeting sequence for the mitochondria outer membrane (FIG. 2A). While the PLPx6 peptide on its own was diffuse in the cytosol (FIG. 2B), upon coexpression with the binder, it was relocalized to mitochondria (FIG. 2C; see also FIG. 6 for controls that binder overexpression does not affect mitochondria shape). Thus the PLPx6/RPB_PLP2_R6 pair retains binding activity in cells. Similar results were obtained for IRPx6-GFP and its cognate binder PXX13_FW6 (FIG. 2D,E).

If individual repeat units on the designed protein engage individual repeat units on the target peptide, binding affinity should increase with increasing the number of repeats. We investigated this with four of our designed systems, in two cases varying the number of protein repeats while keeping the peptide constant, and in the other two, varying the number of peptide repeats while keeping the protein constant. Six-repeat versions of RPB_LRP2 R6 and RPB_PEW2_R6 had higher affinity for eight-repeat LRP and PEW peptides than four-repeat versions without any decrease in specificity. Similarly, six-repeat IYP and PLP peptides had higher affinity for six-repeat versions of the cognate designed repeat proteins (RPB_IYPI_R6, RPB_PLP1_R6) than four-repeat versions. These results are consistent with one to one modular interaction between repeat units on the protein and peptide, and suggest a route to very high binding affinity by simply increasing the number of interacting repeat units. The ability to vary the affinity simply by varying the number of repeats could be useful in many contexts where competitive binding would be advantageous; for example for protein purification by affinity purification, a peptide with a larger number of repeats than that fused to the protein being expressed could be used for elution.

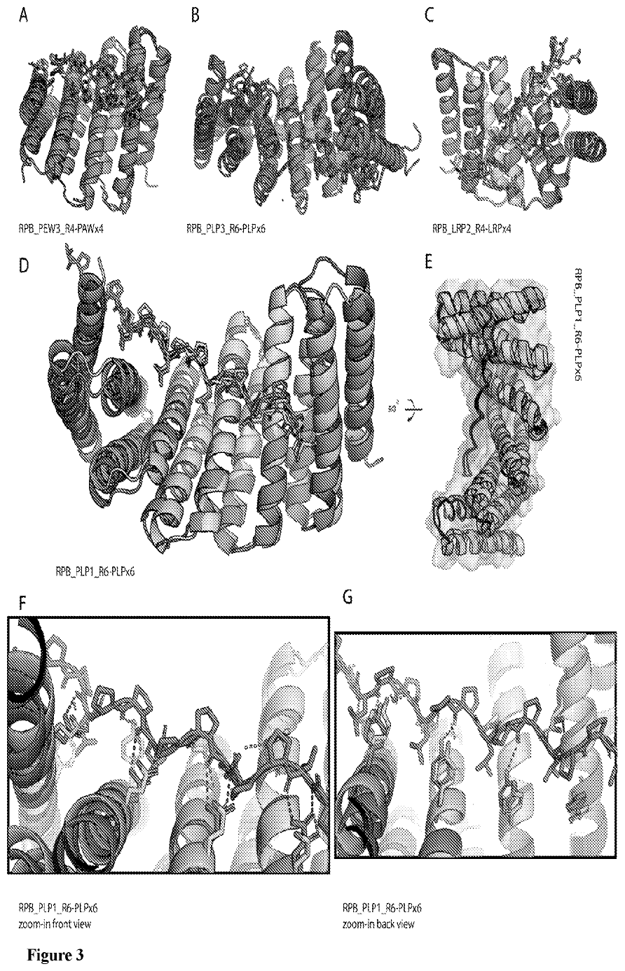

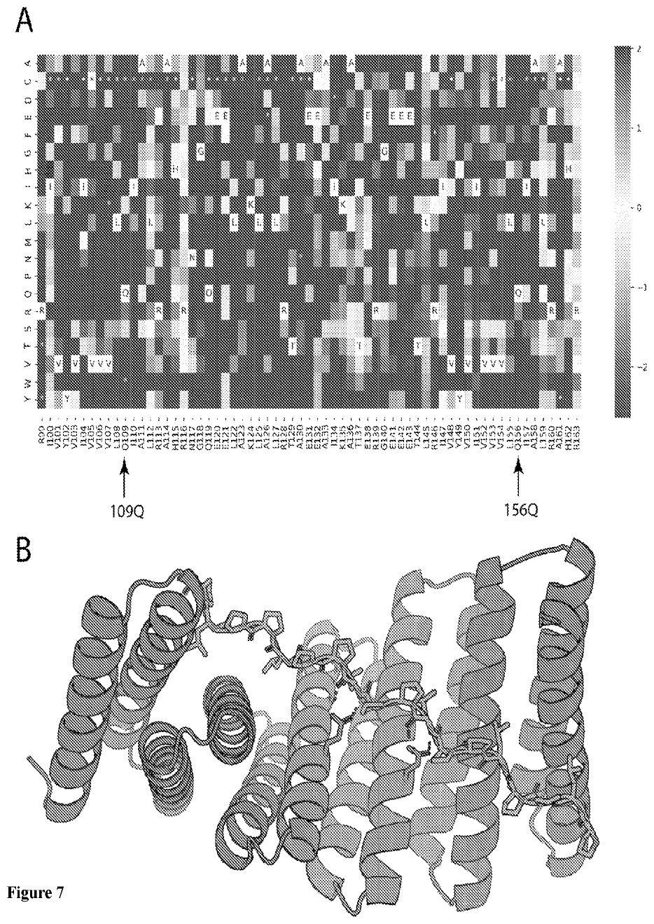

To assess the structural accuracy of our design method, we used X-ray crystallography. We succeeded in obtaining high-resolution co-crystal structures of three first-round designs (RPB_PEW3_R4-PAWx4, RPB_LRP2 R4-LRPx4, RPB PLP3 R6-PLPx6) and one second-round design (RPB_PLP1_R6-PLPx6) (FIG. 3); and a crystal structure of the unbound first-round design RPB_LRP2_R4. In the crystal structure of RPB_PLP3 R6-PLPx6 design, the PLP units fit exactly into the designed curved groove formed by repeating tyrosine, alanine, and tryptophan residues matching the design model with near atomic accuracy, with Cα rmds of 1,70 Å for the binder apo, 2.00 Å for the peptide neighbor interface and 1.64 Å for the whole complex (FIG. 3B). In the co-crystal structure of RPB_PEW3 R4-PAWx4, the PAW units bind to a relatively flat groove formed by repeating histidine residues and glutamine residues as designed (FIG. 3A); the Ca root-mean-square deviation (RMSD) between design model and crystal structure over the repeat protein is 2.08 Å, and the median value of the RMSD to the crystal structure over the peptide and the binding residues in the flexible docking generated ensemble (which converged less well than for the second round designs) is 2.12 Å within 0.03 Å-3.89 Å. For RPB_LRP2 R4 LRPx4, flexible backbone docking converged well with the LRP units sitting in between repeating Glutamine residues and Phenylalanine residues as designed, and the peptide Arginine sidechain sampling two distinct states associated with parallel and antiparallel protein binding modes. The lowest energy docked structure was close to the crystal structure with Cα rmds of 1.15 Å for the binder alone, 0.98 A for the peptide plus protein contacting residues, and 1.16 Å over the entire complex (FIG. 2C). SSM binding interface footprinting results were consistent with the design model and crystal structure (FIG. 7), and a FtoW substitution that increases interactions across the interface substantially increases affinity).

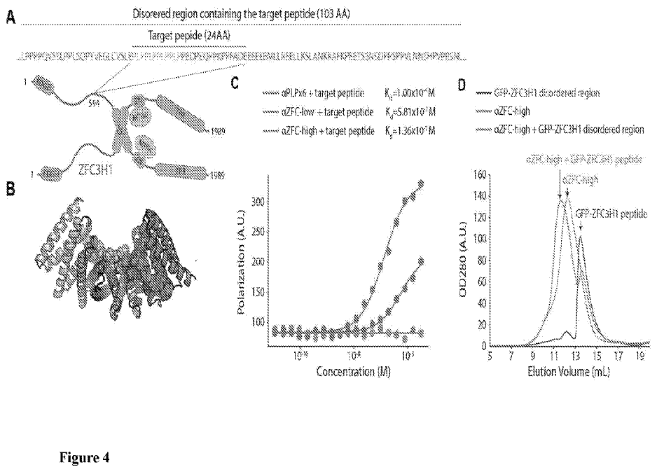

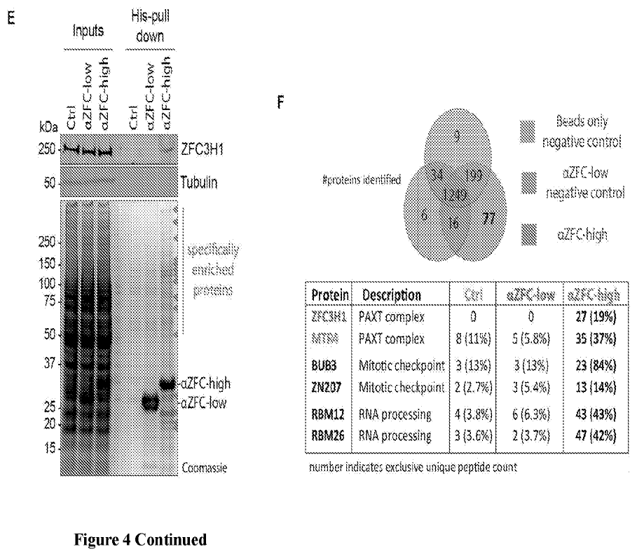

The 2.15A crystal structure of the 2nd round design RPB_PLP1_R6-PLPx6 (SEQ ID NO: 114) highlights key features of the computational design protocol. The PLPx6 (SEQ ID NO: 114) peptide binds to the slightly curved groove primarily through polar interactions from tyrosine, hydrophobic interactions from Valine, and sidechain-backbone bidentate hydrogen bonds from Glutamine exactly as designed (FIG. 3D-4G). The Cα rmds are 1.11 Å for the peptide neighbor interface and 1.81 Å for the binder apo, 1.91 Å for the complex. All interacting side-chains from both the protein side and the peptide side in the computational design model are nearly perfectly recapitulated in the crystal structure. This design has near picomolar binding affinity and high specificity for the PLP target sequence.

We next investigated the specificity of the six designs. The PLPx6 (SEQ ID NO: 114), LRPx6 (SEQ ID NO: 115), PEWx6 (SEQ ID NO: 116), IYPx6 (SEQ ID NO: 117), PKWx6 (SEQ ID NO: 118) binders showed almost complete orthogonality in the 5˜40nM concentration range, with each design binding its cognate designed repeat peptide much more strongly than the other repeat peptides. For example, PLPx6 binds design RPB_PLP1 R6 strongly at 5 nM, but shows no binding signal to design RPB_TYP1_R6 at 40 nM, while PEWx6 binds design RPB_PEW1_R6 but not design RPB_PKW1 R6 at all at 20 nM. Some crosstalk was observed between the PRMx6 (SEQ ID NO: 119) and LRPx6 (SEQ ID NO: 115) binders perhaps involving the arginine residue which makes cation-pi interactions in both designs. We observe similar orthogonality of the interaction between peptide/binder pairs in cells, as the IRPx6 (SEQ ID NO: 120) and PLPx6 (SEQ ID NO: 114) binders specifically direct localization of their cognate peptides to different compartments when coexpressed in the same cells (FIG. 2E, F). By enabling the design of multiple orthogonal protein-peptide pairs, our approach provides a route to probing the effects of localizing different proteins to different locations in the same cell.

As described thus far, our approach enables specific binding of peptides with perfectly repeating structures. To go beyond this limitation and enable targeting of a much wider range of non-repeating peptides, we investigated the redesign of a subset of the peptide repeat unit binding pockets to change their specificity. We broke the symmetry in the designed repetitive binding interface by redesigning both protein and peptide in one or more repeats of six-repeat complexes; the rest of the interface was kept untouched to maintain binding affinity. Following redesign, the peptide backbone conformation was optimized by Monte Carlo resampling and rigid body optimization (see Methods). Designs were selected for experimental characterization as described above, favoring those for which the new design had lower binding energy for the new peptide than the original peptide.

We redesigned the PLPx6 (SEQ ID NO: 114) binder RPB_PLP3_R6 to bind two PEP units in the third and fourth positions (target binding sequence PLPPLPPEPPEPPLPPLP (SEQ ID NO: 106), or more concisely, PLP2PEP2PLP2). The redesigned protein, called RPB_hyb1_R6, bound the redesigned peptide considerably more tightly in Octet™ experiments, while the original design favored the original perfectly repeating sequence, resulting in nearly complete orthogonality. We next designed another hybrid starting from the RPB_IYPI_R6-IYPx6 complex, changing 3 of the IYP units to RYP, generating TYP: RYP3, and redesigning the corresponding binding pockets. The new design, RPB_hyb2_R6, selectively bound the intended cognate target as well. We measured binding of all four proteins against all four peptides, and observed quite high specificity of the designed repeat proteins for their intended peptide targets.

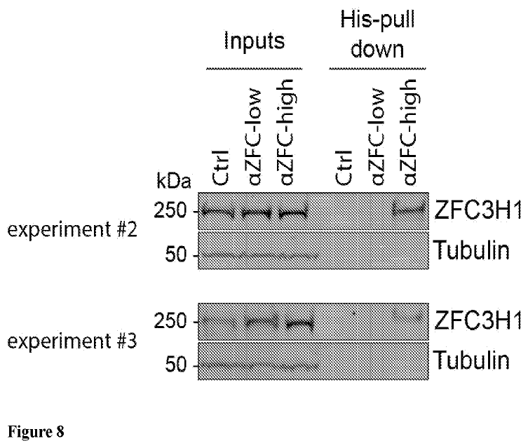

The ability to design hybrid binders against non-repetitive sequences opens the door to the de novo design of binders against endogenous proteins. Intrinsically disordered regions (IDR) are targets of choice, as they have been very difficult to specifically recognize using other approaches, and folding will not interfere with binding. As a proof of concept, we focused on human ZFC3H1, a 200 kDa protein that together with MTR4 forms the heterotetrameric poly(A) tail exosome targeting (PAXT) complex, which directs a subset of long polyadenylated poly(A) RNAs for exosomal degradation (FIG. 4A. We designed binders against ZFC3H residues 594-620 (PLP4PEDPEQPPKPPF (SEQ ID NO: 107)) which lies within a ˜100 residue disordered region (FIG. 4A), by extending both the protein and peptide in the PLPx4 (SEQ ID NO: 109) designed complex. On the peptide side, we kept the (PLP)x4 (SEQ ID NO: 109) backbone fixed, and used Monte Carlo sampling with Ramachandran map biases to model the remaining sequence (PEDPEQPPKPPF (SEQ ID NO: 108)); on the protein side, we extended the PLPx4 design with four additional repeats and designed the interface with each conformer, and selected eight designs for experimental characterization, as described above for the pure repeat binders. Eight designs were expressed, and found to bind the extended target peptide by biolayer interferometry. The two highest affinity designs were further characterized by fluorescence polarization and found to bind the 24 residue target peptide. The one with the highest affinity, named αZFC-high (FIG. 4B), co-eluted with a 103 amino acid segment of the disordered region of ZFC3H1 containing the targeting sequence by Size-Exclusion Chromatography (SEC) (FIG. 4D), demonstrating that the binder can recognize the target peptide in a larger protein context. αZFC-high specifically pulled down the endogenous ZFC3H1 from human cell extracts when assessed by western blot with established antibodies (FIG. 4E, upper panel), in contrast to the lower-affinity binder αZFC-low, which has similar size and surface composition and hence provides a control for non-specific association (see FIG. 8 for replicates, and FIG. 4F for independent identification of ZFC3H1 by mass spectrometry). Mass Spectrometry revealed that MTR4 was enriched in the αZFC-high pull down, demonstrating that the binder can recognize the native PAXT complex in a physiological context. We also detected in the αZFC-high pulldown, but not the αZFC-low pulldown, additional ZFC3H1 partners present in the Bioplex 3.0 interactome in multiple cell lines (33, 34), including BUB3 and ZN207, and multiple RNA binding proteins which likely associate with PAXT-RNA assemblies (FIG. 4F).

Our results demonstrate that by matching superhelical parameters between repeating protein and peptide conformations together with incorporation of specific hydrogen bonding and hydrophobic interactions, new repeat proteins binding repeating peptide sequences with high affinity and specificity can now be designed. The approach should be generalizable to a wide range of repeating peptide structures, and the ability to break symmetry by redesigning individual repeat units opens the door to more general peptide recognition, Our approach complements current efforts at achieving general peptide recognition by redesign of naturally occurring repeat proteins; an advantage of our approach is that a much broader range of protein conformations and binding site geometries can be generated by de novo protein design than by starting with a native protein backbone. Proteins binding repeating or nearly repeating sequences could have applications as affinity reagents for diseases such as Huntington's which are associated with repeat expansions, and rigid fusion of protein modules designed, using the approach described here, to recognize different di, tri and tetra peptide sequences provides an avenue to achieving specific recognition of entirely non-repeating sequences. The ability to design specific binders to proteins containing large disordered regions, demonstrated by the specific pull down of the PAXT complex (FIG. 4), should contribute to delineating the functions of this important but relatively poorly understood class of proteins and reduce reliance on animal immunization to generate antibodies, which can also suffer from reproducibility issues. More generally, our results demonstrate the power of computational protein design for targeting peptides not having rigid three dimensional structures, and as the designed proteins are expressed at quite high levels and very stable, we anticipate that these and further designs for a wider range of target sequences should find broad use in proteomics and other applications requiring specific peptide recognition.

### REFERENCES

1. N. London, D. Movshovitz-Attias, O. Schueler-Furman, The Structural Basis of Peptide-Protein Binding Strategies. Structure. 18, 188-199 (2010).
2. V. Neduva, R. Linding, I. Su-Angrand, A. Stark, F. de Masi, T. J. Gibson, J. Lewis, L. Serrano, R. B. Russell, Systematic Discovery of New Recognition Peptides Mediating Protein Interaction Networks. PLOS Biol. 3, e405 (2005).
3. V. Neduva, R. B. Russell, Peptides mediating interaction networks: new leads at last. Curr. Opin. Biotechnol. 17, 465-471 (2006).
4. P. Emst, A. Plückthun, Advances in the design and engineering of peptide-binding repeat proteins. Biol. Chem. 398, 23-29 (2017).
5. M. A. Andrade, C. Petosa, S. I. O'Donoghue, C. W. Müller, P. Bork, Comparison of ARM and HEAT protein repeats 11Edited by P. E. Wright. J. Mol. Biol. 309, 1-18 (2001).
6. C. Reichen, S. Hansen, C. Forzani, A. Honegger, S. J. Fleishman, T. Zhou, F. Parmeggiani, P. Ernst, C. Madhurantakam, C. Ewald, P. R. E. Mittl, O. Zerbe, D. Baker, A. Caflisch, A. Plückthun, Computationally Designed Armadillo Repeat Proteins for Modular Peptide Recognition. J. Mol. Biol. 428, 4467-4489 (2016).
7. E. Conti, J. Kuriyan, Crystallographic analysis of the specific yet versatile recognition of distinct nuclear localization signals by karyopherin a. Structure. 8, 329-338 (2000).
8. E. Conti, M. Uy, L. Leighton, G. Blobel, J. Kuriyan, Crystallographic Analysis of the Recognition of a Nuclear Localization Signal by the Nuclear Import Factor Karyopberin a. Cell. 94, 193-204 (1998).
9. N. Zeytuni, R. Zarivach, Structural and Functional Discussion of the Tetra-Trico-Peptide Repeat, a Protein Interaction Module. Structure. 20, 397-405 (2012).
10. L. D. D′Andrea, L. Regan, TPR proteins: the versatile helix. Trends Biochem. Sci. 28, 655-662 (2003).
11. P. Ernst. F. Zosel, C. Reichen, D. Nettels, B. Schuler, A. Plückthun, Structure-Guided Design of a Peptide Lock for Modular Peptide Binders. ACS Chem. Biol. 15, 457-468 (2020).
12. R. F. Alford, A. Leaver-Fay, J. R. Jeliazkov, M. J. O'Meara, F. P. DiMaio, H. Park, M. V. Shapovalov, P. D. Renfrew, V. K. Mulligan, K. Kappel, J. W. Labonte, M. S. Pacella, R. Bonneau, P. Bradley, R. L. Dunbrack, R. Das, D. Baker, B. Kuhlman, T. Kortemme, J. J. Gray, The Rosetta All-Atom Energy Function for Macromolecular Modeling and Design. J. Chem. Theory Comput. 13, 3031-3048 (2017).
13. S. Hansen, D. Tremmel, C. Madhurantakam, C. Reichen, P. R. E. Mittl, A. Plückthun, Structure and Energetic Contributions of a Designed Modular Peptide-Binding Protein with Picomolar Affinity. J. Am. Chem. Soc. 138, 3526-3532 (2016).
14. C. Reichen, S. Hansen, A. Plückthun, Modular peptide binding: From a comparison of natural binders to designed armadillo repeat proteins. J. Struct. Biol. 185, 147-162 (2014).
15. J. A. Cross, M. S. Chegkazi, R. A. Steiner, D. N. Woolfson, M. P. Dodding, Fragment-linking peptide design yields a high-affinity ligand for microtubule-based transport. Cell Chem. Biol. 28, 1347-1355.e5 (2021).
16. P. J. Fleming, G. D. Rose, Do all backbone polar groups in proteins form hydrogen bonds? Protein Sci. 14, 1911-1917 (2005).
17. T. J. Brunette, F. Parmeggiani, P.-S. Huang, G. Bhabha, D. C. Ekiert, S. E. Tsutakawa, G. L. Hura, J. A. Tainer, D. Baker, Exploring the repeat protein universe through computational protein design. Nature. 528, 580-584 (2015).
18. L. Shimoni, J. P. Glusker, Hydrogen bonding motifs of protein side chains: descriptions of binding of arginine and amide groups. Protein Sci. Publ. Protein Soc. 4, 65-74 (1995).
19. J. A. Fallas, G. Ueda, W. Sheffler, V. Nguyen, D. E. McNamara, B. Sankaran, J. H. Pereira, F. Parmeggiani, T. J. Brunette, D. Cascio, T. R. Yeates, P. Zwart, D. Baker, Computational design of self-assembling cyclic protein homo-oligomers. Nat. Chem. 9, 353-360 (2017).
20. J. K. Leman, B. D. Weitzner, S. M. Lewis, J. Adolf-Bryfogle, N. Alam, R. F. Alford, M. Aprahamian, D. Baker, K. A. Barlow, P. Barth, B. Basanta, B. J. Bender, K. Blacklock, J. Bonet, S. E. Boyken, P. Bradley, C. Bystroff, P. Conway, S. Cooper, B. E. Correia, B. Coventry, R. Das, R. M. De Jong, F. DiMaio, L. Dsilva, R. Dunbrack, A. S. Ford, B. Frenz, D. Y. Fu, C. Geniesse, L. Goldschmidt, R. Gowthaman, J. J. Gray, D. Gront, S. Guffy, S. Horowitz, P.-S. Huang, T. Huber, T. M. Jacobs, J. R. Jeliazkov, D. K. Johnson, K. Kappel, J. Karanicolas, H. Khakzad, K. R. Khar, S. D. Khare, F. Khatib, A. Khramushin, I. C. King, R. Kleffner, B. Koepnick, T. Kortemme, G. Kuenze, B. Kuhlman, D. Kuroda, J. W. Labonte, J. K. Lai, G. Lapidoth, A. Leaver-Fay, S. Lindert, T. Linsky, N. London, J. H. Lubin, S. Lyskov, J. Maguire, L. Malmström, E. Marcos, O. Marcu, N. A. Marze, J. Meiler, R. Moretti, V. K. Mulligan, S. Nerli, C. Nom, S. Ó′Conchúir, N. Ollikainen, S. Ovchinnikov, M. S. Pacella, X. Pan, H. Park, R. E. Pavlovicz, M. Pethe, B. G. Pierce, K. B. Pilla, B. Raveh, P. D. Renfrew, S. S. R. Burman, A. Rubenstein, M. F. Sauer, A. Scheck, W. Schief, O. Schueler-Furman, Y. Sedan, A. M. Sevy, N. G. Sgourakis, L. Shi, J. B. Siegel, D.-A. Silva, S. Smith, Y. Song, A. Stein, M. Szegedy, F. D. Teets, S. B. Thyme, R. Y.-R. Wang, A. Watkins, L. Zimmerman, R. Bonneau, Macromolecular modeling and design in Rosetta: recent methods and frameworks. Nat. Methods. 17, 665-680 (2020).
21. D. Kuroda, J. J. Gray, Shape complementarity and hydrogen bond preferences in protein-protein interfaces: implications for antibody modeling and protein-protein docking. Bioinformatics. 32, 2451-2456 (2016).
22. B. Coventry, D. Baker, Protein sequence optimization with a pairwise decomposable penalty for buried unsatisfied hydrogen bonds. PLOS Comput. Biol. 17, e1008061 (2021).
23. E. T. Boder, K. D. Wittrup, Yeast surface display for screening combinatorial polypeptide libraries. Nat. Biotechnol. 15, 553-557 (1997).
24. T. Kortemme, D. Baker, A simple physical model for binding energy hot spots in protein-protein complexes. Proc. Natl. Acad. Sci. 99, 14116-14121 (2002).
25. T. Kortemme, D. E. Kim, D. Baker, Computational Alanine Scanning of Protein-Protein Interfaces. Sci. STKE. 2004, pl2-pl2 (2004).
26. G. L. Hura, H. Budworth, K. N. Dyer, R. P. Rambo, M. Hammel, C. T. McMurray, J. A.

Tainer, Comprehensive macromolecular conformations mapped by quantitative SAXS analyses. Nat. Methods. 10, 453-454 (2013).
27. Robust, high-throughput solution structural analyses by small angle X-ray scattering (SAXS) | Nature Methods, (available at https://www.nature.com/articles/nmeth.1353).
28. R. M. P. Siloto, R. J. Weselake, Site saturation mutagenesis: Methods and applications in protein engineering. Biocatal. Agric. Biotechnol. 1, 181-189 (2012).
29. J. Helma, M. C. Cardoso, S. Muyldermans, H. Leonhardt, Nanobodies and recombinant binders in cell biology. J. Cell Biol. 209, 633-644 (2015).
30. S. Moutel, N. Bery, V. Bernard, L. Keller, E. Lemesre, A. de Marco, L. Ligat, J.-C. Rain, G. Favre, A. Olichon, F. Perez, NaLi-H1: A universal synthetic library of humanized nanobodies providing highly functional antibodies and intrabodies. eLife. 5, c16228 (2016).
31. A.-E. Foucher, L. Touat-Todeschini, A. B. Juarez-Martinez, A. Rakitch, H. Laroussi, C. Karczewski, S. Acajjaoui, M. Soler-López, S. Cusack, C. D. Mackereth, A. Verdel, J. Kadlec, Structural analysis of Red1 as a conserved scaffold of the RNA-targeting MTREC/PAXT complex. Nat. Commun. 13, 4969 (2022).
32. N. Meola, M. Domanski, E. Karadoulama, Y. Chen, C. Gentil, D. Pultz, K. Vitting-Seerup, S. Lykke-Andersen, J. S. Andersen, A. Sandelin, T. H. Jensen, Identification of a Nuclear Exosome Decay Pathway for Processed Transcripts. Mol. Cell. 64, 520-533 (2016).
33. D. K. Schweppe, E. L. Huttlin, J. W. Harper, S. P. Gygi, BioPlex Display: An Interactive Suite for Large-Scale AP-MS Protein-Protein Interaction Data. J. Proteome Res. 17, 722-726 (2018).
34. E. L. Huttlin, R. J. Bruckner, J. Navarrete-Perea, J. R. Cannon, K. Baltier, F. Gebreab, M. P. Gygi, A. Thornock, G. Zarraga, S. Tam, J. Szpyt, B. M. Gassaway, A. Panov, H. Parzen, S. Fu, A. Golbazi, E. Maenpaa, K. Stricker, S. Guba Thakurta, T. Zhang, R. Rad, J. Pan, D. P. Nusinow, J. A. Paulo, D. K. Schweppe, L. P. Vaites, J. W. Harper, S. P. Gygi, Dual proteome-scale networks reveal cell-specific remodeling of the human interactome. Cell. 184, 3022-3040.e28 (2021).
32. N. Meola, M. Domanski, E. Karadoulama, Y. Chen, C. Gentil, D. Pultz, K. Vitting-Seerup, S. Lykke-Andersen, J.S. Andersen, A. Sandelin, T.H. Jensen, Identification of a Nuclear Exosome Decay Pathway for Processed Transcripts. Mol. Cell. 64, 520-533 (2016).

### Materials and Methods

### Synthetic Gene Constructs

For both the first and second round designs, a His-tag containing TEV protease cleavage site and short linkers were added to the N-terminus of protein sequences. For the protein lacking a Tryptophan residue, a single Tryptophan was added to the short N-terminal linker following the TEV protease cleavage site to help with protein concentration quantification by A280. The protein sequence along with linker (MGSSHHHHHHHHSSGGSGGLNDIFEAQKIEWHEGGSGGSENLYFQSG (SEQ ID NO: 110) or LEHHHHHH (SEQ ID NO: 111)) was reverse translated into DNA using a custom python script that attempts to maximize host-specific codon adaptation index¹ and IDT synthesize-ability, which includes optimizing whole gene and local GC content as well as removing repetitive sequences. Finally, a TAATCA stop codon was appended to the end of each gene. Genes were delivered cloned into pET-29b+between NdeI/XhoI restriction sites. For the second round designs, the designed amino acid sequences were inserted into pET-29b+between NdeI/XhoI restriction sites directly.

### Protein Expression and Purification

Proteins were transformed into Lemo21 (DE3) E. coli from New England Biolabs (NEB) and then expressed as 50 ml cultures in 250 ml flasks using Studiers M2 autoinduction media with 50 μg/mL kanamycin. The cultures were either grown at 37° C. for ˜6-8 hours and then ˜18° C. overnight (˜14 hours) or at 37° C. the entire time ˜14 hours. Cells were pelleted at 4,000g for 10 minutes, after which the supernatant was discarded. Pellets were resuspended in 30 ml lysis buffer (25 mM Tris HCl pH 8, 150 mM NaCl, 30 mM imidazole, 1 mM PMSF, 0.75% CHAPS, 1 mM DNase, 10 mM Lysozyme, with Thermo Scientific Pierce protease inhibitor tablet). Cell suspensions were lysed by microfluidizer or sonication, and the lysate was clarified at 20,000g for ˜30 minutes. The His-tagged proteins were bound to Ni-NTA resin (Qiagen) during gravity flow and washed with a wash buffer (25 mM Tris HCl pH 8, 150 mM NaCl, 30 mM imidazole). Protein was eluted with an elution buffer (25 mM Tris HCl pH 8, 150 mM NaCl, 300 mM imidazole). For the first round designs, the His-tag was removed by TEV cleavage, followed by IMAC purification to remove TEV protease. The flowthrough was collected and concentrated prior to further purification by SEC/FPLC on a Superdex™ 200 increase 10/300 GL column in TBS (25 mM Tris pH 8.0, 150 mM NaCl).

### Circular Dichroism

Circular dichroism spectra were measured with an AVIV Model 420 DC or Jasco J-1500 CD spectrometer. Samples were 0.25 mg/mL in TBS (25 mM Tris pH 8.0, 150 mM NaCl), and a 1-mm path length cuvette was used. The CD signal was converted to mean residue ellipticity by dividing the raw spectra by N×C×L×10, where N is the number of residues, C is the concentration of protein, and L is the path length (0.1 cm).

Size Exclusion Chromatography with Multi-Angle Light Scattering

Purified samples after the initial SEC run, samples were pooled then concentrated or diluted as needed to a final concentration of 2 mg/mL. 100 μL of each sample was then run through a high-performance liquid chromatography system (Agilent) using a Superdex™ 200 10/300 GL column. These fractionation runs were coupled to a multi-angle light scattering detector (Wyatt) in order to determine the absolute molecular weights for each designed protein as described previously².

### Small Angle X-Ray Scattering

Small-Angle X-ray Scattering (SAXS) was collected at the SIBYLS High Throughput SAXS Advanced Light Source in Berkeley, California³. Beam exposures of 0.3 s for 10.2 s resulted in 33 frames per sample. Data was collected at low (˜1.5 mg/mL) and high (˜2-3 mg/mL) protein concentrations in SAXS buffer (25 mM Tris pH 8.0, 150 mM NaCl, 2% glycerol). The sibyls website (“SAXS FrameSlice” n.d.) was used to analyze the data for high and low centration samples and average the best dataset. If there was obvious aggregation over the 33 frames, only the data points before aggregation arose were used in the Gunier region, otherwise, all data was included for the Gunier region. All data was used for Porod and Wide regions. The averaged file was used with scatter. jar to remove data points with outlier residuals in the Gunier region. Finally, the data was truncated at 0.25 q. This dataset was then compared to the predicted SAXS profile based on the design model using the FoxS SAXS server, and volatility ratio (Vr) was calculated to quantify how well the predicted and data matched the experimental data. Proteins with Vr of less than 2.5 were considered to be folded to the designed quaternary shape.

### Biolayer Interferometry

Biolayer interferometry binding data were collected in an Octet™ RED96 (ForteBio) and processed using the instrument's integrated software. To measure the affinity of peptide binders, N-terminal biotinylated (biotin-Ahx) target peptides with a short linker (GGS) were loaded onto streptavidin-coated biosensors (SA ForteBio) at 50-100 nM in binding buffer (10 mM HEPES (pH 7.4), 150 mM NaCl, 3 mM EDTA, 0.05% surfactant P20, 0.5% non-fat dry milk) for 120 s. Analyte proteins were diluted from concentrated stocks into the binding buffer. After baseline measurement in the binding buffer alone, the binding kinetics were monitored by dipping the biosensors in wells containing the target protein at the indicated concentration (association step) and then dipping the sensors back into baseline/buffer (dissociation).

### Yeast Surface Display

S. cerevisiae EBY 100 strain cultures were grown in C-Trp-Ura media and induced in SGCAA media following the protocol in (reference). Cells were washed with PBSF (PBS with 1% BSA) and labeled with biotinylated designed proteins using two labeling methods, with-avidity and without-avidity labeling. For the with-avidity method, the cells were incubated with biotinylated RBD, together with anti-c-Myc fluorescein isothiocyanate (FITC, Miltenyi Biotech) and streptavidin-phycoerythrin (SAPE, ThermoFisher). The concentration of SAPE in the with-avidity method was used at ¼ concentration of the biotinylated RBD. The with-avidity method was used in the first few rounds of screening against the repeat peptide library to fish out weak binder candidates. For the without-avidity method, the cells were firstly incubated with biotinylated designed proteins, washed, secondarily labeled with SAPE and FITC.

### Crystallography

### Crystallographic Data Collection and Refinement Statistics

TABLE 4

| | RPB_PEW3_R4- | | RPB_PLP3_R6- | | RPB_LRP2_R4- | | RPB_PLP1_R6- | |
|---|---|---|---|---|---|---|---|---|
| | PAWx4 | | PLPx6 | | LRPx4 | | PLPx6 | |
| Data | | | | | | | | |
| collection | | | | | | | | |
| Beamline | APS 23-ID-D | | APS 23-ID-D | | APS 23-ID-B | | APS 23-ID-B | |
| Wavelength | 1.033 | | 1.033 | | 1.033 | | 1.033 | |
| Space group | P2₁2₁2₁ | | I422 | | P3₂21 | | R32 | |
| Cell | | | | | | | | |
| dimensions | | | | | | | | |
| a, b, c (Å) | 45.4, 67.6, 71.3 | | 124.5, 124.5, 105.0 | | 70.8, 70.8, 128.2 | | 99.4, 99.4, 173.2 | |
| α, β, γ (°) | 90.0, 90.0, 90.0 | | 90.0, 90.0, 90.0 | | 90.0, 90,0, 120.0 | | 90.0, 90.0, 120.0 | |
| Resolution (Å) | 38.3-2.70 | (2.80-2.70)¹ | 45.1-2.68 | (2.78-2.68)¹ | 35.4-3.18 | (3.92-3.18)¹ | 37.7-2.15 | (2.21-2.15)¹ |
| Total | 39,005 | | 155,310 | | 64,884 | | 186,166 | |
| Observations | | | | | | | | |
| Unique | 6,383 | | 11,891 | | 6,621 | | 18,220 | |
| reflections | | | | | | | | |
| Redundancy | 6.1 | (6.4)¹ | 13.1 | (13.3)¹ | 9.8 | (10.2)¹ | 10.2 | (10.5)¹ |
| Completeness | 99.6 | (99.8)¹ | 92.1 | (77.4)¹ | 89.1 | (67.9)¹ | 99.9 | (100)¹ |
| (%) | | | | | | | | |
| Rₘₑₐₛ | 0.08 | (1.14)¹ | 0.11 | (2.51)¹ | 0.08 | (3.38)¹ | 0.06 | (2.45)¹ |
| I/σ(I) | 12.0 | (1.27)¹ | 18.5 | (1.21)¹ | 14.1 | (0.69)¹ | 18.1 | (0.85)¹ |
| CC_{1/2} | 1.00 | (0.81)¹ | 1.00 | (0.51)¹ | 1.00 | (0.69)¹ | 1.00 | (0.45)¹ |

| | | | | |
|---|---|---|---|---|
| Refinement | | | | |
| Resolution | 38.3-2.70 | 45.1-2.68 | 35.4-3.18 | 37.7-2.15 |
| (Å) | | | | |
| Reflections | 6,057 | 10,412 | 5,357 | 15,249 |
| (work) | | | | |
| Reflections | 316 | 547 | 294 | 1,672 |
| (test) | | | | |
| R_{work}/R_{free} | 24.5/27.0 | 22.6/27.7 | 20.7/25.6 | 23.2/27.6 |
| (%) | | | | |
| Average B- | 114.9 | 86.9 | 162.9 | 86.9 |
| factor (Å²) | | | | |
| No. atoms | | | | |
| Protein | 1,502 | 2,375 | 1,372 | 2,172 |
| Peptide | 104 | 139 | 104 | 240 |
| Water | 0 | 0 | 0 | 7 |
| Ramachandran² | | | | |
| Favored (%) | 98.0 | 98.4 | 97.2 | 99.3 |
| Outliers (%) | 0.0 | 0.0 | 0.6 | 0.0 |
| Rotamers² | | | | |
| Favored (%) | 96.20 | 98.8 | 94.08 | 97.1 |
| Outliers (%) | 0.0 | 0.0 | 0.0 | 0.0 |
| R.m.s. | | | | |
| deviations | | | | |
| Bond lengths | 0.002 | 0.002 | 0.004 | 0.002 |
| (Å) | | | | |
| Bond angles | 0.41 | 0.49 | 0.62 | 0.41 |
| (°) | | | | |
| Molprobity² | | | | |
| Molprobity | 1.10 | 1.38 | 1.78 | 1.01 |
| Score | | | | |
| Percentile | 100^{th} | 100^{th} | 100^{th} | 100^{th} |
| Clashscore | 3.12 | 6.99 | 13.35 | 3.06 |
| Percentile | 100^{th} | 99^{th} | 95^{th} | 99^{th} |
| PDB ID | 7UDJ | 7UE2 | 7UDK | 7UDL |

TABLE 5

| | RPB_PLP1_R6-PLPx6, | | RPB_PLP1_R6-PLPx6, | | | |
|---|---|---|---|---|---|---|
| | alternative | | alternative | | RPB_LRP2_R4, | |
| | conformation 1 | | conformation 2 | | pseudopolymeric | |
| Beamline | APS 23-ID-B | | APS 23-ID-B | | APS 23-ID-B | |
| Wavelength | 1.033 | | 1.033 | | 1.033 | |
| Space group | P22₁2₁ | | P22₁2₁ | | P3₂21 | |
| Cell | | | | | | |
| dimensions | | | | | | |
| a, b, c (Å) | 54.4, 80.3, 154.4 | | 80.4, 86.5, 110.2 | | 72.6, 72.6, 127.3 | |
| α, β, γ (°) | 90.0, 90.0, 90.0 | | 90.0, 90.0, 90.0 | | 90.0, 90.0, 120.0 | |
| Resolution | 43.2-2.65 | (2.72-2.65)¹ | 43.2-2.45 | (2.51-2.45)¹ | 44.7-2.50 | (2.57-2.50)¹ |
| (Å) | | | | | | |
| Total | 135,662 | | 191,404 | | 139,040 | |
| Observations | | | | | | |
| Unique | 20,350 | | 28,936 | | 13,974 | |
| reflections | | | | | | |
| Redundancy | 6.7 | (6.7)¹ | 6.6 | (6.7)¹ | 9.9 | (9.8)¹ |
| Completeness | 99.8 | (100.0)¹ | 99.9 | (100.0)¹ | 100.0 | (100.0)¹ |
| (%) | | | | | | |
| Rₘₑₐₛ | 0.10 | (2.11)¹ | 0.07 | (1.85)¹ | 0.07 | (2.18)¹ |
| I/σ(I) | 10.9 | (0.9)¹ | 14.0 | (1.2)¹ | 18.6 | (1.0)¹ |
| CC_{1/2} | 1.00 | (0.57)¹ | 1.00 | (0.49)¹ | 1.00 | (0.48)¹ |

| | | | |
|---|---|---|---|
| Refinement | | | |
| Resolution | 43.2-2.65 | 43.2-2.45 | 44.7-2.50 |
| (Å) | | | |
| Reflections | 16,916 | 25,465 | 12,425 |
| (work) | | | |
| Reflections | 1,167 | 1,466 | 659 |
| (test) | | | |
| R_{work}/R_{free} | 26.0/29.9 | 23.5/26.7 | 19.3/21.9 |
| (%) | | | |
| Average B- | 113.7 | 91.4 | 89.7 |
| factor (Å²) | | | |
| No. atoms | | | |
| Protein | 4,492 | 4,565 | 1,399 |
| Peptide | 88 | 0 | 0 |
| Water | 0 | 0 | 0 |
| Ramachandran² | | | |
| Favored (%) | 98.9 | 99.1 | 98.2 |
| Outliers (%) | 0.0 | 0.0 | 0.0 |
| Rotamers² | | | |
| Favored (%) | 99.2 | 97.6 | 97.2 |
| Outliers (%) | 0.4 | 0.9 | 0.7 |
| R.m.s. | | | |
| deviations | | | |
| Bond lengths | 0.001 | 0.002 | 0.012 |
| (Å) | | | |
| Bond angles | 0.294 | 0.339 | 1.353 |
| (°) | | | |
| Molprobity² | | | |
| Molprobity | 1.04 | 0.94 | 1.28 |
| Percentile | 100^{th} | 100^{th} | 100^{th} |
| Clashscore | 2.58 | 1.84 | 5.24 |
| Percentile | 100^{th} | 100^{th} | 99^{th} |
| PDB ID | 7UDM | 7UDN | 7UDO |

¹Numbers in parentheses refer to the highest resolution shell
²As reported by MolProbity⁴.

### Crystallization and Structure Determination for RPB_PEW3_R4-PAWx4

Purified RPB_PEW3_R4 protein+PAWx4 (SEQ ID NO: 121) peptide at a concentration of 36 mg/mL was used to conduct sitting drop, vapor-diffusion crystallization trials using the JCSG Core I-IV screens (NeXtal Biotechnologies). Crystals of RPB_PEW3_R4-PAWx4 grew from drops consisting of 100 nL protein plus 100 nL of a reservoir solution consisting of 0.1 M MES pH 5.0, 30% (w/v) PEG 6K at 4° C., and were cryoprotected by supplementing the reservoir solution with 5% ethylene glycol. Native 10 diffraction data was collected at APS beamline 23-ID-D, indexed to P212: 21 and reduced using XDS5 (Tables 4-5). The structure was phased by molecular replacement using Phaser™. A set of ˜50 lowest energy predicted models from Rosetta™ were used as search models. Several of these models gave clear solutions, which were adjusted in Coot™ and refined using Phenix™ Model refinement in P2: 2121 initially resulted in unacceptably high values for R_{free}-R_{work}. Refinement was therefore first performed in lower symmetry space groups (P1 and P2₁). In the late stages of refinement, these P1 and P2₁ models were refined against the P2₁2₁2₁, which ultimately yielded acceptable, albeit somewhat higher R-factors.

### Crystallization and Structure Determination for RPB_PLP3_R6-PLPx6

Purified RPB_PLP3_R6 protein+PLPx4 (SEQ ID NO: 109) peptide at a concentration of 70 mg/mL was used to conduct sitting drop, vapor-diffusion crystallization trials using the JCSG Core I-IV screens (NeXtal Biotechnologies). Crystals of RPB_PLP3_R6-PLPx6 grew from drops consisting of 100 nL protein plus 100 nL of a reservoir solution consisting of 2.4 M (NH4)2SO4, 0.1 M Na3Cit pH4 at 18° C., and were cryoprotected by supplementing the reservoir solution with 2.2M sodium malonate pH4. Native diffraction data was collected at APS beamline 23-ID-D, indexed to 1422 and reduced using XDS5 (Tables 4-5). The structure was phased by molecular replacement using Phaser™. A set of ˜28 lowest energy predicted models from Rosetta™ were used as search models. Several of these models gave clear solutions, which were adjusted in Coot™ and refined using Phenix™

### Crystallization and Structure Determination for RPB_LRP2 R4-LRPx4

Purified RPB_LRP2_R4 protein+LRPx4 (SEQ ID NO: 122) peptide at a concentration of 21.4 mg/mL was used to conduct sitting drop, vapor-diffusion crystallization trials using the JCSG Core I-IV screens (NeXtal Biotechnologies). Crystals of RPB_LRP2_R4-LRPx4 grew from drops consisting of 100 nL protein plus 100 nL of a reservoir solution consisting of 0.1 M HEPES pH7, 10% (w/v) PEG6000 at 18° C., and were cryoprotected by supplementing the reservoir solution with 25% Ethylene glycol. Native diffraction data was collected at APS beamline 23-ID-B, indexed to P32 2 1 and reduced using XDS5 (Tables 4-5). The structure was phased by molecular replacement using Phaser™. The coordinates of apo RPB_LRP2_R4 from the proteolyzed/filament structure were used as a search model. The resulting model was adjusted in Coot™ and refined using Phenix™. Like the apo structure, this crystal structure of RPB_LRP2 R4 also contained “infinitely long filaments in the crystal, this time with peptide bound.

### Crystallization and Structure Determination for RPB_PLP1_R6-PLPx6

Purified RPB_PLP1_R6 protein+PLPx6 (SEQ ID NO: 114) peptide at a concentration of 143 mg/mL was used to conduct sitting drop, vapor-diffusion crystallization trials using the JCSG Core I-IV screens (NeXtal Biotechnologies). Crystals of RPB_PLP1 R6-PLPx6 grew from drops consisting of 100 nL protein plus 100 nL of a reservoir solution consisting of 0.2 M NaCl, 20% (w/v) PEG3350 at 4° C., and were cryoprotected by supplementing the reservoir solution with 15% Ethylene glycol. Native diffraction data was collected at APS beamline 23-ID-B, indexed to H32 and reduced using XDS⁵ (Tables 4-5). The structure was phased by molecular replacement using Phaser™. A set of ˜230 lowest energy predicted models from Rosetta™ were used as search models. Several of these models gave clear solutions, which were adjusted in Coot™ and refined using Phenix™. In the later stages of refinement, two copies of the 6xPLP (SEQ ID NO: 114) peptide were built into clearly defined electron density in the asymmetric unit. The first copy adopts the expected location based on the design, and makes the designed interactions with RPB_PLP1_R6. The density for this peptide and the final atomic model (19 amino acid residues) are slightly longer than the peptide used in crystallization (18 residues); this is likely due to “slippage”/misregistration of the peptide relative to the R6PO11 in many unit cells, resulting in density longer than the peptide itself. A second copy of the peptide lies across a 2-fold symmetry axis at ˜50% occupancy, resulting in the superposition of this peptide with a symmetry-derived copy of itself running in the opposite direction. Despite this, the locations of each Pro/Leu side chain unit was reasonably well defined. However, it seems unlikely that the binding of the peptide at this second site would occur readily in solution.

### Crystallization and Structure Determination for RPB_PLP1_R6, Alternative Conformation 1

Purified RPB_PLP1_R6 protein+PLPx6 (SEQ ID NO: 114) peptide at a concentration of 166 mg/mL was used to conduct sitting drop, vapor-diffusion crystallization trials using the JCSG Core I-IV screens (NeXtal Biotechnologies). Crystals of RPB_PLP1_R6-PLPx6 grew from drops consisting of 100 nL protein plus 100 nL of a reservoir solution consisting of 0.02 M CaCl2, 30% (v/v) MPD, 0.1 M NaAcet pH 4.6 at 18° C., and were cryoprotected by supplementing the reservoir solution with 5% MPD. Native diffraction data was collected at APS beamline 23-ID-B, indexed to P22121 and reduced using XDS5 (Tables 4-5). The structure was phased by molecular replacement using Phaser™, using the coordinates for R6PO11 (alternative conformation 1) as a search model. The model was adjusted in Coot™ and refined using Phenix™. In the later stages of refinement, one copy of the 6xPLP peptide was model at a site of crystal contact, where it is sandwiched between adjacent subunits in a way that is likely only bound in the crystal lattice

### Crystallization and Structure Determination for RPB_PLP1 R6, Alternative Conformation 2

Purified RPB_PLP1_R6 protein+PLPx6 (SEQ ID NO: 114) peptide at a concentration of 166 mg/mL was used to conduct sitting drop, vapor-diffusion crystallization trials using the JCSG Core I-IV screens (NeXtal Biotechnologies). Crystals of RPB_PLP1 R6-PLPx6 grew from drops consisting of 100 nL protein plus 100 nL of a reservoir solution consisting of 40% (v/v) MPD, 0.1 M Na Phos Cit pH 4.2 at 18° C., and were cryoprotected by supplementing the reservoir solution. Native diffraction data was collected at APS beamline 23-ID-B, indexed to P22121 and reduced using XDS5 (Tables 4-5). Initial attempts to phase by molecular replacement using Phaser™ and ˜500 predicted models from Rosetta™ and RoseTTAfold™ failed to yield any clear solutions. Similarly, several thousand truncations of these models (containing all combinations of 1, 2, 3, 4, or 5 of the 6 repeat units), also failed to give clear solutions. To try to identify correct but low scoring solutions in the output of these trials, we ran SHELXE autobuilding and density modification on a large number of these potential solutions. Ultimately, we were able to identify an MR solution with 2 of 6 repeats correctly placed that allowed the autobuilding of a polyalanine model and an interpretable map, which could be further improved by iterative rounds of rebuilding in Coot™ and refinement using Phenix™. Ultimately, the final model revealed that in this crystal form and a similar crystallization condition (RPB_PLP1_R6, alternative conformation 1, above), RPB_PLP1_R6 adopted an alternative fold.

### Crystallization and Structure Determination for RPB_LRP2 R4

Purified RPB_LRP2_R4-LRPx4 protein at a concentration of 33 mg/mL was used to conduct sitting drop, vapor-diffusion crystallization trials using the JCSG Core I-IV screens (NeXtal Biotechnologies). Crystals of RPB_LRP2_R4 grew from drops consisting of 100 nL protein plus 100 nL of a reservoir solution consisting of 0.2 M K2HPO4, 20% (w/v) PEG 3350 at 18° C., and were cryoprotected by supplementing the reservoir solution with 15% ethylene glycol. Native diffraction data was collected at APS beamline 23-ID-B, indexed to P 32 2 1 and reduced using XDS5 (Tables 4-5). The structure was phased by molecular replacement using Phaser™. A set of ˜50 lowest energy predicted models from Rosetta™, as well as a variety of truncated models, were used as search models. Several of these models gave clear solutions, which were adjusted in Coot™ and refined using Phenix™. Four helical repeat modules were present in the asymmetric unit. However, unexpectedly, side chain density for all four repeats were very similar to one another and matched the sequence of the internal helical repeats, but not the N- and C-terminal capping repeats, which are slightly different from the internal ones. In addition, these four repeat units pack tightly against adjacent, symmetry-related molecules such that they form an “infinitely long” repeat protein running throughout the crystal. Careful examination of the the junction between each repeat unit revealed no clear breaks in electron density; the density for the backbone is continuous through the asymmetric unit, and continuous with the symmetry related molecules near the N- and C-terminus of the molecule in the asymmetric unit. Rather than truly forming an infinitely long polymer, we suspect that proteolytic cleavage of the RPB_LRP2_R4 (either during purification or crystallization led to the removal of the N- and/or C-terminal caps in many molecules, which could allow the internal repeats from separate molecules to polymerize to form fibers in the crystal. Heterogeneity in these cleavage products and how they assemble into the crystal lattice (misregistration) could consequently explain the “continuous” filaments of this repeat protein that we observe in these crystals.

### Cell Studies

### Plasmids

For expression in cells, constructs were synthesized by Genescript and cloned into a modified pUC57 plasmid (Genscript) allowing mammalian expression under a EF1a promoter. Target peptides were cloned as C-terminal fusions with a linker (GAGAGAGRP (SEQ ID N): 123)) followed by EGFP (SEQ ID NO: 124). Binders were expressed as fusions with the first 34 residues of the Mas70p protein (Mito-tag), shown to efficiently relocalise proteins to mitochondria in mammalian cells⁹ in N-terminal and with mScarlet™ in C-terminal¹⁰. Plasmids encoding the GFP-tagged peptide and the mScarlet™-tagged binder were then cotransfected into cells.

Alternatively, for in vivo demonstration of the multiplexed binding between different peptides and their cognate binders (FIG. 2F,G), bicistronic plasmids were generated expressing the binder flanked with a Mito-tag™ (respectively, PEX-tag, the first 66 residues of human PEX3, targeting to peroxisomes¹¹) followed by a stop codon, then an Internal ribosome entry site (IRES) sequence and the target peptide tagged with EGFP (SEQ ID NO: 124) (respectively mScarlet™). Cells were then cotransfected with both bicistronic plasmids to express all four proteins.

### Cells

U2OS cells (ATCC, HTB-96) were cultured in DMEM (Corning) supplemented with 10% fetal bovine serum (Gibco) and 1% Pen/Strep (Gibco) at 37° C. with 5% CO₂. Cells were transfected with Lipofectamine™ 3000 (Invitrogen) according to the manufacturer's instructions and imaged after 1 day of expression.

Live-cell imaging

For live cell imaging (FIG. 2), U2OS cells were plated on glass-bottom dishes (World Precision Instruments, FD35) coated with fibronectin (Sigma, F1141, 50 μg/ml in PBS), for 1 hours at 37° C. DMEM-10% serum. Medium was then changed to Leibovitz's L-15 medium (Gibco) supplemented with 20 mM HEPES (Gibco) for live cell imaging. Imaging was performed onto a custom spinning disk confocal instrument composed of Nikon Ti stand equipped with perfect focus system, a fast Z piezo stage (ASI) and a PLAN Apo Lambda 1.45 NA 100X objective, and a spinning disk head (Yokogawa CSUXI). Images were recorded with a Photometrics Prime™ 95B back-illuminated sCMOS camera run in pseudo global shutter mode and synchronized with the spinning disk wheel. Excitation was provided by 488, 561 lasers (all Coherent OBIS mounted in a Cairn laser launch) and imaged using dedicated single bandpass filters for each channel mounted on a Caim Optospin wheel (Chroma 525/50 for GFP and Chroma 595/50 for mScarlet™). To enable fast 4D acquisitions, an FPGA module (National Instrument sbRIO-9637 running custom code) was used for hardware-based synchronization of the instrument, in particular to ensure that the piezo z stage moved only during the readout period of the sCMOS camera. Temperature was kept at 37° C. using a temperature control chamber (MicroscopeHeaters.Com, Brighton UK). System was operated by Metamorph.

### Immunofluorescence

For immunofluorescence of mitochondria (FIG. 6), U2OS FlpIn™ Trex cells were spread on glass-bottom dishes coated with fibronectin as above. Cells were washed with PBS then fixed in 4% PFA for 20 minutes at room temperature. Following fixation, cells were washed in with PBS and then permeabilized with 0.1% Triton X-100 in PBS for 5 minutes at room temperature. Cells were washed again with PBS and blocked in 1% BSA in PBS for 15 minutes. Cells were then incubated with TOM20 antibody (santa cruz), diluted in 1% BSA in PBS, for 1 hour at room temperature. Cells were washed 3 times with PBS and then incubated with DAPI and anti-mouse Alexa™ Fluor 488, diluted at 1/500e in 1% BSA in PBS, for one hour at room temperature. Cells were washed a final 3 times in PBS and then imaged using the spinning disk confocal described above.

Pull Down of Endogenous Proteins from Extracts Using Designed Binders

For pull down of endogenous ZFC3H1 from human cell extracts, HeLa FlpIn Trex cells were lysed in lysis buffer (25 mM HEPES, 150 mM NaCl, 0.5% Tx100, 0.5% NP-40, 20 mM imidazole, pH 7.4 supplemented with Roche EDTA free protease inhibitor tablets). Lysate was incubated on ice for 10 minutes to continue lysis and then were spun at 4000×g for 15 minutes at 4° C. The supernatant was incubated with pre-washed Ni-NTA agarose (Qiagen, 30210 318/AV/01) for 1 hour with rocking at 4° C. to remove/reduce proteins in the lysate that bind to the resin non-specifically. For each condition, 50 μl of fresh Ni-NTA agarose resin was washed twice in lysis buffer. Equimolar amounts of purified his-tagged binder, or as a control an equal volume of buffer, was added to the Ni-NTA agarose. The pre-cleared HeLa lysate was split evenly between the 3 conditions. An input was taken of each condition, and the tubes were incubated for 2 hours at 4° C. with rocking. Beads were then washed twice in lysis buffer and twice in wash buffer (25 mM HEPES, 150 mM NaCl, 20 mM imidazole pH 7.4). Proteins were then eluted from the beads in elution buffer (25 mM HEPES, 150 mM NaCl, 500 mM imidazole, pH 7.4). Inputs and elutions were ran on a NuPage 3-8% Tris-Acetate gel (Invitrogen, EA0375) and transferred to a nitrocellulose membrane using the iBlot system (ThermoFischer). Membranes were blocked in 5% (w/v) milk in TBS-TWEEN™ (10 mM Tris-HCl, 120 mM NaCl, 1% (w/v) TWEEN20, pH 7.4) for 30 mins at room temperature with gentle shaking. Rabbit anti-ZFC3H1 (Sigma, HPA007151, used at 1:250) and Mouse anti-alpha tubulin 488 (Clone DMA1, Sigma T6199, directly labelled with Abberior® STAR 488, NHS ester leading to a 4.5 dye/antibody degree of labelling, and used at 0.1 μg/mL final concentration) were diluted 1% (w/v) milk in TBS-TWEEN and incubated with the membrane overnight at 4° C. with gentle shaking. The membrane was washed 3x in TBS-TWEEN then incubated with goat anti-Rabbit Alexa 555 (Invitrogen, A32732, 1:2000) for one hour at room temperature with gentle shaking. The membrane was washed twice with TBS-TWEEN and a final wash with TBS-TWEEN with 0.001% SDS. Membranes were imaged using a ChemiDoc™ system (BioRad). Alternatively, the same samples were analyzed using 4-12% Bis-Tris gels (Invitrogen NP0323BOX) and stained with Instant blue coomassie stain (Sigma ISBIL). Note that αZFC-high is also able to pull down endogenous ZFC3H1 from human cell extracts when 50 mM rather than 150 mM NaCl was used in all buffers (FIG. 8C).

### Mass Spectrometry

Each line of the polyacrylamide gel presented in FIG. 4C was cut into six pieces and prepared for mass spectrometric analysis by manual in situ enzymatic digestion (the gel area containing the binder was omitted from the analysis to avoid saturation of the detector by overabundance of binder peptides). Briefly, the excised protein gel pieces were placed in a well of a 96-well microtiter plate and destained with 50% v/v acetonitrile and 50 mM ammonium bicarbonate, reduced with 10 mM DTT, and alkylated with 55 mM iodoacetamide. After alkylation, proteins were digested with 6 ng/μL. Trypsin (Promega, UK) overnight at 37° C. The resulting peptides were extracted in 2% v/v formic acid, 2% v/v acetonitrile. The digest was analyzed by nano-scale capillary LC-MS/MS using a Ultimate U3000 HPLC (ThermoScientific Dionex, San Jose, USA) to deliver a flow of approximately 300 nL/min. A C18 Acclaim PepMap™100 5 μm, 100 μm×20 mm nano Viper (ThermoScientific Dionex, San Jose, USA), trapped the peptides prior to separation on a C18 Acclaim PepMap100 3 μm, 75 μm×150 mm nano Viper™ (ThermoScientific Dionex, San Jose, USA). Peptides were eluted with a gradient of acetonitrile. The analytical column outlet was directly interfaced via a modified nano-flow electrospray ionisation source, with a hybrid dual pressure linear ion trap mass spectrometer (Orbitrap™ Velos, ThermoScientific, San Jose, USA). Data dependent analysis was carried out, using a resolution of 30,000 for the full MS spectrum, followed by ten MS/MS spectra in the linear ion trap. MS spectra were collected over a m/z range of 300-2000. MS/MS scans were collected using a threshold energy of 35 for collision induced dissociation. LC-MS/MS data were then searched against an in house protein sequence database, containing Swiss-Prot and the protein constructs specific to the experiment, using the Mascot search engine program (Matrix Science, UK) 18 Database search parameters were set with a precursor tolerance of 5 ppm and a fragment ion mass tolerance of 0.8 Da. Two missed enzyme cleavages were allowed and variable modifications for oxidized methionine, carbamidomethyl cysteine, pyroglutamic acid, phosphorylated serine, threonine, tyrosine, tert-butyloxycarbonyl-lysine, norbornene-lysine and prop-2-yn-1-yloxycarbonyl-lysine were included. MS/MS data were validated using the Scaffold program (Proteome Software Inc., USA) 19. All data were additionally interrogated manually. To generate the Venn diagram in FIG. 4D, we considered a threshold of minimum 5 peptides to consider that a protein had been identified.

### Design Methods

DHR Scaffolds generation

Each Designed Helical Repeat (DHR) scaffold is formed by a helix-loop-helix-loop topology that is repeated four or more times¹². The helices range from 18 to 30 residues and the loops from 3 to 4 residues. The DHR design process goes through backbone design, sequence design and computational validation by energy landscape exploration. To match the peptides the designs were required to have a twist (omega) between 0.6 and 1.0 radians, a radius of 0 to 13 A and a rise between 0 and 10 A. The geometry of a repeat protein can be described by the radius of the super-helix, the axial displacement and the twist¹³.

The backbone is designed using Rosetta™ fragment assembly guided by motifs¹⁴. Backbone coordinates are built up through 3,200 Monte Carlo fragment assembly steps with fragments harvested from a non-redundant set of structures from the PDB. Following each fragment insertion, the rigid body transform is propagated to the downstream repeats. The score that guides fragment assembly is composed of Van der Waal interactions, packing, backbone dihedral angles, and Residue-Pair-Transform (RPX) motifs¹⁴. RPX motifs are a fast way to measure the full-atom hydrophobic packability of the backbone prior to assigning side chains. After design, backbones are screened for native-like features. The loops are required to be within 0.4A of a naturally occurring loop or rebuilt. Structures with helices above 0.14 Å appear bent and kinked and are discarded. And poorly packed structures where <4 helices are in contact with each-other are filtered.

Sequence is designed using Rosetta™ for each backbone that passes filtering. Design begins in a symmetric mode where each repeat is identical using the RepeatProteinRelax mover. Core residues are restricted to be hydrophobic and surface residues hydrophilic using the layer design task operators. Sequence is biased toward natural proteins with similar local structure using the structure profile mover. After the symmetrical design is complete the N-terminal and C-terminal repeats are redesigned to eliminate exposed hydrophobics. Designs with poor core packing as measured by Rosetta™ holes <0.5 are then filtered¹⁵.

The designs are computationally validated using the Rosetta™ ab initio structure prediction on Rosetta@Home™. Rosetta™ ab initio verifies that the design is lower energy state than the thousands of alternatives conformations sampled. Simulating a protein using Rosetta@Home™ can take several days on hundreds of CPUs. To speed this up we used machine learning to filter designs that were most likely to fail¹³.

### Backbone Generation of Curved Repeat Protein Monomers in Poly-Proline II Conformation

A second round of designs was made to ensure the distance between helices match the 10.9 Å. distance between prolines in the Poly-Proline II. To design these backbones we used AtomPair constraints between the first helix of each repeat. The atom pair constraints were set to 10.9 Å with a tolerance of 0.5 Å. For these designs we found the topologies that most efficiently produced structures that matched the Atom Pair constraints bad a helix length of 20 or 21 residues and a loop range of three residues.

### Peptide Binders Design

### Modular Peptide Docking and Hashing

To construct hash tables storing the pre-computed privileged residue interactions, we first survey the non-redundant PDB database and extract the intended interacting residues as seeds. For each seeding interaction residue pairs, random perturbations were applied to search for alternative relative conformations of the interacting residues. In the case of the sidechain-backbone bidentate interactions, the backbone residues were applied a random rigid body perturbations with a random set of euler angles drawing from a normal distribution with 0° as the mean and 60° as the standard deviation, as well as a random set of translation distances in 3D space drawing from a normal distribution with 0 A as the mean and 1 A as the standard deviation. At the same time, the backbone torsion angles @ and VP of the backbone residue were randomly modified to values draw from a ramachandran density plot based on structures from PDB database. The transformed set of residues losing the intended interactions were discarded. The transformed residues keeping the interactions will be collected. Then the side chains of the sidechain residues were replaced with all reasonable rotamers, to further diversify the samples of the sets of interacting residues. Finally, the geometry relationship of each set of residues keeping the intended interactions were subjected to an 8D hash function (6D rigid body transformation plus two torsion angles), and represented with a 64 bit unsigned integer as the key of an entry in the hash table. The identity and the side chain torsion angles (Xs) of the side chain residues were treated as the value of the entry in the hash table. The similar processes were carried out to build different hash tables for various interactions, with minor alterations. For example, for pi-pi and cation-pi interactions, only a 6D hash function was used, because there is no need for the perturbation and consideration of the backbone torsions. For ASN, GLN, ASP or GLU interacting two residues on the backbone, a 10D hash table was applied for representing the geometry relationship, and in these cases, the geometries of the N—H and C═O groups on the backbone were treated as 5D rays.

To sample repeat peptides matching the superhelical parameters of the DHRs, we randomly generate a set of backbone torsion angles φ and ψ, for example, [φ₁, ψ₁, φ₂, ψ₂, φ₃, ψ₃] for repeats of tri-peptide. If there any pair of φ and ψ angles get a high Rosetta Ramachandran score above the threshold −0.5, it means that this pair of torsion angles are likely to introduce intra-peptide steric clashes, and we will randomly regenerate a new pair of φ and ψ angles until they are reasonable according to the Rosetta Ramachandran score. Next, we will set the backbone torsion angles of the repeat peptide using this set of o and v angles repetitively across the 8-repeats. And we will calculate the superhelical parameters using the 3D coordinates of adjacent repeat units of the repeat peptide. The repeat peptides matching the superhelical parameters of any one of the curated DHRs, will be saved for the docking step.

To dock cognate repeat proteins and repeat peptides, with matching superbelical parameters, they are first aligned to the z axis by their own superhelical axes. Next step, a 2D grid search (rotation around and translation along the z axis) were carried out to sample compatible positions of the repeat peptide in the binding groove of the repeat protein. Once a reasonable dock is generated without steric clash, the relevant hash function will be used to iterate through all potential peptide-protein interacting residue sets, to calculate the hash keys. If a hash key exists in the hash table, the interacting side chain identities and torsion angles would be pulled out immediately and installed on all equivalent positions of this repeat peptide-repeat protein docking conformation. The docked peptide-DHR pair will be saved for the interface design step if the peptide-DHR hydrogen-bond interactions are satisfied.

### Peptide Binding Interface Design

If a single dock was accepted with the designed repetitive peptide-DHR hydrogen-bond, the peptide was first trimmed to the exact same repeat number as the DHR (e.g., 4-repeat or 6-repeat). After that, for both peptide and DHR sides, each amino acid was set linked to its corresponding amino acids on the same position in each repeat unit. This was to make sure all the following design steps would be carried out with the exact same symmetry inside of both the DHR and peptide.

During our design cycles, the interface neighbor distance is set as 9 Å as the whole designable range around the DHR-peptide binding interface, and 11 Å as the whole minimization range. Three rounds of full hydrophobic fastdesign¹⁴ followed by hydropathic fastdesign were carried out, with each hydrophobic or hydrophilic fastdesign repeating twice. The Rosetta score function beta_nov16 was chosen in all design cycles. In the produced complex, the peptide itself with an averaged score (three calculations were carried out) larger than 20.0 or the complex scored larger than-10.0 were rejected directly.

After the preliminary design was done, we carried out two types of sanity checks to further optimize the designed peptide sequence, as well as the designed DHR interface. Specifically, for the peptide side, in the tri-peptide repeat units, every two amino acids other than Proline were scanned for a possible mutation to all twenty amino acids except cysteine, unless a certain originally designed peptide amino acid is making the hashed sidechain-backbone hydrogen-bond, or sidechain-sidechain hydrogen-bond, or sidechain-sidechain-backbone hydrogen-bond with the DHR interface. DDG (binding energy for the peptide-DHR. complex) was compared before and after this peptide side mutation; and the mutation was accepted if the delta DDG (DDG_after-DDG_before) was larger than 1.0. Similarly, we also checked the designed DHR interface by mutation. The whole DHR was scanned. For the designed hydrophobic amino acids which were originally hydrophilic, a delta DDG of-5.0 was set as the threshold to be accepted as a necessary design which made enough binding contribution. For the designed hydropathic amino acids, a delta DDG of-2.0 was used as threshold.

For experimental characterization, we selected designed complexes with near ideal bidentate hydrogen bonds between protein and peptide, favorable protein-peptide interaction energies (DDG<=−35.0), interface shape complementary (Iface_SCval>=0.65), tolerable interface unsatisfied hydrogen bonds (Iface_HbondsUnsatBB<=2, Iface_HbondsUnsatSC <=4) and low peptide apo energies (ScoreRes_chainB<=0.9).

### Forward Docking

As for the selected designed complexes from our round-two experiments, forward docking was performed to ensure the specificity in silico. For each designed complex, 10,000 arbitrary peptide conformations were generated as above, using the designed sequence. The same docking protocol was conducted as described in the docking age, against the untouched designed DHR. Fastrelax¹⁷ was then performed for the 10,000 docks, and the DDG vs. peptide backbone RMSD was plotted to check the convergence of the complex. Only the “converged” complexes were selected for experimental characterization, e.g., i) peptide backbone RMSD<2.0 Å among the top 20 designs with lowest DDG during Forward Docking and ii) the averaged peptide backbone of the top 20 designs was close to the original design model (RMSD<1.5 Å).

SSM library preparation

We carried out the site saturation mutagenesis (SSM) studies for some of the designed peptide-protein binding pairs to gain better understanding of the peptide binding modes, and to search for improved peptide binders. For each designed repeat protein, we ordered a SSM library covering the central span of 65 amino acids within the whole repeat protein, due to the chip DNA size limitation. This span roughly equals one and half repeating units, across three helices. The chip synthesized DNA oligos for the SSM library then amplified and transformed to EBY 100 yeast together with a linearized pETCON3 vector including the encoding regions of the rest of the designed repeat protein. Each SSM library was subject to an expression sort first, in which the low-quality sequences due to chip synthesize defects or recombination errors were filtered out. The collected yeast population, which successfully expresses the designed repeat protein mutants, will be re-grown, and be subject to the further round of peptide binding sorts. The next-generation sequencing results of this yeast population will also serve as the reference data for SSM analysis. The next round of without-avidity peptide binding sorts used various concentrations of the target peptide, depending on the initial peptide binding abilities, ranging from 1 nM to 1000 nM. The peptide-bound yeast populations were collected and sequenced by using Illumina NextSeq kit. The mutants were identified and compared to the mutants in the expression libraries. The enrichment analysis was carried out to identify the beneficial mutants and provide information for interpreting the peptide binding modes. For each mutant, its enrichment value is calculated by dividing its ratio in peptide-bound population by its ratio in expression population. The enrichment value is then subject to a log 10 transformation, and plotted in heatmaps for the SSM analysis.

### Design of Binders Against Endogenous Targets

To evaluate which endogenous proteins could be currently targeted with our method (FIG. 4), we developed a python code to search databases for subsequences matching permutations of the set of amino acid triplets we designed binders for in this study (i.e LRP, PEW, PLP, IYP, PKW, IRP, LRT, LRN, LRQ, RRN, PSR, and PRQ). This code can be accessed freely (https://github.com/tjs23/prot_pep_scan). We then ranked all outputs to find the longest subsequence possible, and manually inspected the candidates to find subsequences landing in disordered regions. Doing this analysis on the human proteome suggested that ZFC3H1 could be a good target for two main reasons: 1) this protein possess the sequence (PLP)x4 (SEQ ID NO: 109) within a large disordered domain, with downstream sequence (PEDPEQPPKPPF (SEQ ID NO: 108)) within the reach of our binder design method and 2) the protein is well studied, and, in particular, commercial, highly specific and validated antibodies exist against it.

TABLE 7
First-round experimental characterization summary. It is clearly
shown that among the binders, most of them bound peptides with
sequences similar to those targeted but not the same; and peptides
with 3 residue repeat units were targeted more successfully (19
in total) than those with 2 residue repeat units (2 in total).

| | Design | Experimental | di-peptide | Tri-peptide |
|---|---|---|---|---|
| Design ID | target | target | target | target |
| PXX02 | PSD | PEW | | 1 |
| PXX10 | PMP | PWP/PAW | | 2 |
| PXX12 | ADP | LRP/LRT | | 2 |
| PXX27 | PDH | PDW | | 1 |
| PXX28 | PMP | PAW/PKW | | 2 |
| PXX45 | PD | DW | 1 | |
| CP06 | VRP | LRT/INR | | 2 |
| CP10 | IMP | IRP | | |
| CP25 | PSM | LRP/MYP | | 2 |
| CP33 | PKL | PRM/PRY/PKW | | 3 |
| KP02 | DI | PAN/PEW | | 2 |
| KP09 | RP | LRA | | 1 |
| KP22 | DP | PEW/EW | 1 | 1 |
| summary | | | 2 | 19 |

TABLE 8
Second-round experimental characterization summary. 54 second-
round designed protein-peptide pairs were tested in total. 42
of the designed proteins were solubely expressed in E coli,
25 were monomerically dispersed by Size Exclusion Chromatography
(SEC), and 16 designed bound their targets with considerably
higher affinity and specificity than in the first round.

| | | | | Affinity < |
|---|---|---|---|---|
| | | | Binding to the | uM by yeast |
| | | | designed target | surface |
| | Expressed | | by yeast surface | display & |
| Tested | well | Monomeric | display | Octet ™, BLI |
| 54 | 42 | 25 | 19 | 16 |

### REFERENCES

1. Sharp P. M., Li W., The codon adaptation index-a measure of directional synonymous codon usage bias, and its potential applications, Nucleic Acids Research, 15 (3), 281-1295 (1987).
2. Fallas, J., Ueda, G., Sheffler, W. et al. Computational design of self-assembling cyclic protein homo-oligomers. Nature Chem 9, 353-360 (2017).
3. Dyer, K. N., Hammel, M., Rambo, R. P., Tsutakawa, S. E., Rodic, I., Classen, S., Tainer, J. A., & Hura, G. L. High-throughput SAXS for the characterization of biomolecules in solution: a practical approach. Methods in molecular biology (Clifton, N.J.), 1091, 245-258 (2014).
4. Williams, C. J. et al. MolProbity: More and better reference data for improved all-atom structure validation. Protein Sci. 27, 293-315 (2018).
5. Kabsch, W. XDS. Acta Crystallogr. D Biol. Crystallogr. 66, 125-132 (2010).
6. McCoy, A. J. et al. Phaser crystallographic software. J. Appl. Crystallogr. 40, 658-674 (2007)
7. Emsley, P., Lohkamp, B., Scott, W. G. & Cowtan, K. Features and development of Coot. Acta Crystallogr. D Biol. Crystallogr. 66, 486-501 (2010).
8. Adams, P. D. et al. PHENIX: a comprehensive Python-based system for macromolecular structure solution. Acta Crystallogr. D Biol. Crystallogr. 66, 213-221 (2010).
9. Kessels, M. M., & Qualmann, B. Syndapins integrate N-WASP in receptor-mediated endocytosis. The EMBO journal, 21 (22), 6083-6094 (2002).
10. Bindels, D., Haarbosch, L., van Weeren, L. et al. mScarlet: a bright monomeric red fluorescent protein for cellular imaging. Nat Methods 14, 53-56 (2017).
11. Fakieh, M. H., Drake, P. J., Lacey, J., Munck, J. M., Motley, A. M., & Hettema, E. H. Intra-ER sorting of the peroxisomal membrane protein Pex3 relies on its luminal domain. Biology open, 2 (8), 829-837 (2013).
12. Brunette, T J et al. Exploring the repeat protein universe through computational protein design. Nature 528, 580-584 (2015).
13. Brunette, T J et al. Modular repeat protein sculpting using rigid helical junctions. Proc. Natl. Acad. Sci. U.S.A 117, 8870-8875 (2020).
14. Fallas, J. A. et al. Computational design of self-assembling cyclic protein homo-oligomers. Nat. Chem. 9, 353-360 (2017).
15. Sheffler, W. & Baker, D. RosettaHoles: rapid assessment of protein core packing for structure prediction, refinement, design, and validation. Protein Sci. 18, 229-239 (2009).
16. Bradley, P., Misura, K. M. S. & Baker, D. Toward high-resolution de novo structure prediction for small proteins. Science 309, 1868-1871 (2005).
17. Tyka M D, Keedy D A, André I, et al. Alternate states of proteins revealed by detailed energy landscape mapping. J Mol Biol. 405 (2): 607-618 (2011).
18. Perkins, D. N., Pappin, D. J. C., Creasy, D. M. & Cottrell, J. S. Probability-based protein identification by searching sequence databases using mass spectrometry data. Electrophoresis. 20, 3551-3567 (1999).
19. Keller, A., Nesvizhskii, A. I., Kolker, E. & Aebersold, R. Empirical statistical model to estimate the accuracy of peptide identifications made by MS/MS and database search. Anal. Chem. 74, 5383-5392 (2002).

1. A polypeptide comprising or consisting of an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 1-105.

2. The polypeptide of claim 1 comprising an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 1-105.

3. The polypeptide of claim 1 comprising an amino acid sequence at least 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 1-105.

4. The polypeptide of claim 1 comprising an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO: 1-105.

5. The polypeptide of claim 1 comprising an amino acid sequence at least 90%, 91%, 92%, 93%, 94%, 95%, 96%, 97%, 98%, 99%, or 100% identical to the amino acid sequence selected from the group consisting of SEQ ID NO: 1-105.

6. The polypeptide of claim 1 comprising an amino acid sequence at 95%, 96%, 97%, 98%, 99%, or 100% identical to the amino acid sequence selected from the group consisting of SEQ ID NO: 1-105.

7. The polypeptide of claim 1, wherein any N-terminal methionine residue is deleted.

8. The polypeptide of claim 1, wherein any N-terminal methionine residue is present.

9. The polypeptide of claim 1, wherein 1, 2, 3, 4, or 5 amino acids at the N-terminus and/or C-terminus of the polypeptide may be absent.

10. The polypeptide of claim 1, wherein substitutions relative to the reference sequence are conservative amino acid substitutions.

11. The polypeptide of claim 1, further comprising one or more functional domains.

12. A nucleic acid encoding the polypeptide of claim 1.

13. An expression vector comprising the nucleic acid of claim 12 operatively linked to a suitable control sequence.

14. A host cell comprising the expression vector of claim 13.

15. A pharmaceutical composition comprising
(a) the polypeptide of claim 1; and
(b) pharmaceutically acceptable carrier.

16. A library comprising a plurality of different polypeptides of claim 1.

17. A method for the polypeptide, of claim 1 for any suitable purpose, including but not limited to use as synthetic peptide ligands for receptor activation.
